(19) United States
(12) Patent Application Publication
(10) Pub. No.: US 2025/0170124 A1
(43) Pub. Date: May 29, 2025

(54) METHODS OF DIAGNOSING AND TREATING NEURODEGENERATIVE DISEASES

(71) Applicant: The General Hospital Corporation, Boston, MA (US)

(72) Inventors: Catherine Cahill, Arlington, MA (US); Changning Wang, Melrose, MA (US); Jack Rogers, Charlestown, MA (US)

(21) Appl. No.: 18/843,569

(22) PCT Filed: Mar. 21, 2023
(86) PCT No.: PCT/US2023/015742

§ 371 Date: Sep. 3, 2024

Related U.S. Application Data
(60) Provisional application No. 63/322,350, filed on Mar. 22, 2022.

(51) Int. Cl. A61K 31/496; A61K 31/235; A61K 31/40; A61K 31/42; A61K 31/4365; A61K 31/4748; A61K 31/4985; A61K 31/53; A61K 31/554; A61K 51/04; G01N 33/68

(52) U.S. Cl. CPC A61K 31/496; A61K 31/235; A61K 31/40; A61K 31/42; A61K 31/4365; A61K 31/4748; A61K 31/4985; A61K 31/53; A61K 31/554; A61K 51/0402; A61K 51/0446; A61K 51/0453; A61K 51/0455; A61K 51/0459; A61K 51/0461; A61K 51/0468; G01N 33/6896; G01N 2800/28

(57) ABSTRACT

This disclosure provides compounds, pharmaceutical compositions, imaging compositions and methods useful for the diagnosis and/or treatment of neurodegenerative diseases. In particular, this disclosure provides compounds, including radiolabeled compounds, compositions, and methods useful for the diagnosis and/or treatment of neurodegenerative diseases associated with a-synuclein aggregation, such as Parkinson's disease, dementia with Lewy bodies, multiple systems atrophy or prodromal REM sleep behavior disorder.

### CLAIM OF PRIORITY

This application claims the benefit of U.S. Patent Application Ser. No. 63/322,350 filed on Mar. 22, 2022. The contents of which is hereby incorporated by reference in its entirety.

### TECHNICAL FIELD

This disclosure provides compounds, compositions, and methods useful for the diagnosis and/or treatment of neurodegenerative diseases. In particular, this disclosure provides compounds, compositions, and methods useful for the diagnosis and/or treatment of neurodegenerative diseases associated with α-synuclein aggregation, such as Parkinson's disease.

### BACKGROUND

Synucleinopathies such as Parkinson's disease (PD) and dementia with Lewy bodies (DLB) are a class of neurodegenerative diseases characterized by the abnormal accumulation of aggregates of α-synuclein protein in the cytoplasm of selective populations of neurons and glia. These aggregates are the main component of both Lewy bodies, the defining histological feature of PD and DLB. Such aggregates have been shown to accompany neuronal damage (Ubhi et al. Trends Neurosci. 2011 November; 34(11): 581-590). Deposition of aggregates of synuclein in neurons and glia suggests that a common pathogenic mechanism may exist for these disorders.

PD is one of the most common movement disorders and DLB is the second most common cause of degenerative dementia. A causative link between α-synuclein amyloid formation and disease progression is supported by the findings that gene duplications and familial mutations that increase amyloid load also cause early-onset PD, and more than 90% of sporadic PD patients stain positive for α-synuclein deposits. Cardinal clinical features of PD include slowness of movement, decrease of amplitude and speed, as well as bradykinesia, rest tremor, and rigidity (Urbizu and Beyer. Int J Mol Sci. 2020 July; 21(13): 4718).

There is currently no cure for neurodegenerative diseases such as PD. Current treatments focus on the relief of symptoms and improvements to the quality of life. The value of current therapies for Parkinson's disease (PD) and other neurodegenerative diseases is limited by the fact they do little, if anything, to modify the underlying progression of the disease. Therapies with disease modifying properties, e.g., therapies that are neuroprotective, neurorestorative or slow, halt or even reverse disease progression, will be of great value. Current approaches attempt prevent and/or inhibit α-synuclein aggregation and/or cytotoxicity. Posiphen is the only α-synuclein inhibitor that has been used to treat PD, but it lacks the specificity and potency needed to prevent Lewy body formation. Consequently, there remains a need for α-synuclein blockers that can be used to treat PD.

### SUMMARY

The present application is based, in part, on the surprising discovery of compounds that can block the 5′ untranslated region (5′UTR) of α-synuclein mRNA. These compounds can be used to prevent the gait disturbances seen in PD by limiting the formation of Lewy bodies in the mid-brain and cortex of patients.

Accordingly, some embodiments provide a method for treating an α-synuclein associated disease or disorder in a subject in need thereof, comprising administering to the subject a therapeutically effective amount of a compound of selected from the group consisting of:

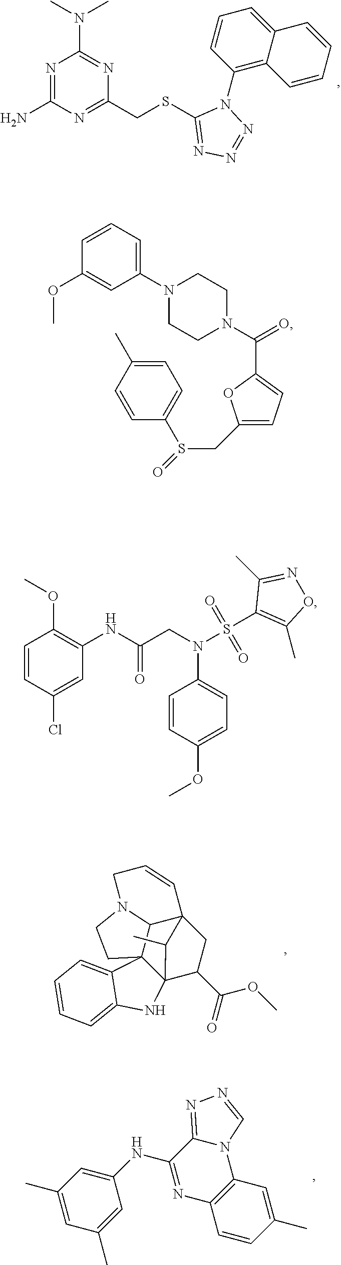

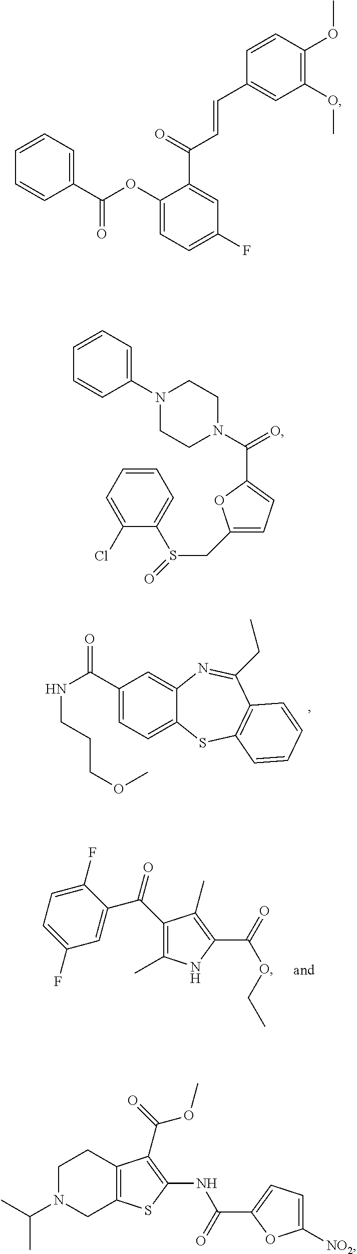

or a pharmaceutically acceptable salt of any of the foregoing.

Some embodiments provide a method for treating an α-synuclein associated disease or disorder in a subject in need thereof, comprising administering to the subject a therapeutically effective amount of a compound of selected from the group consisting of:

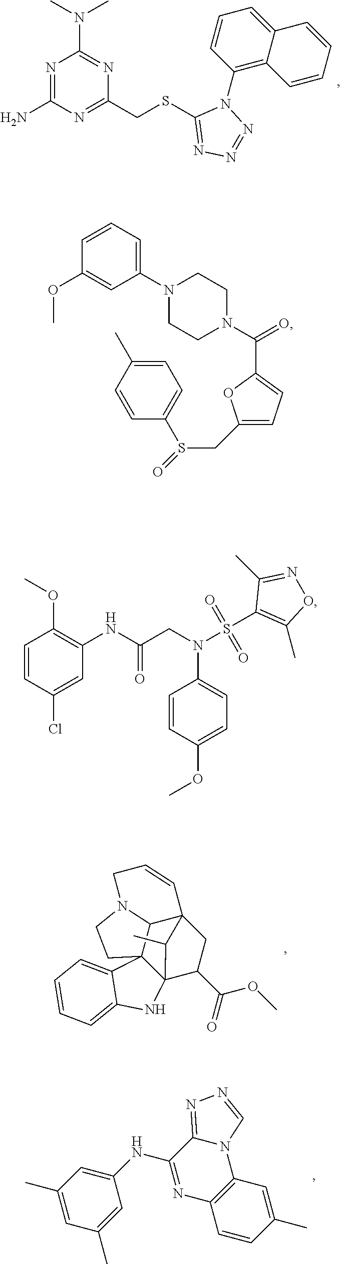

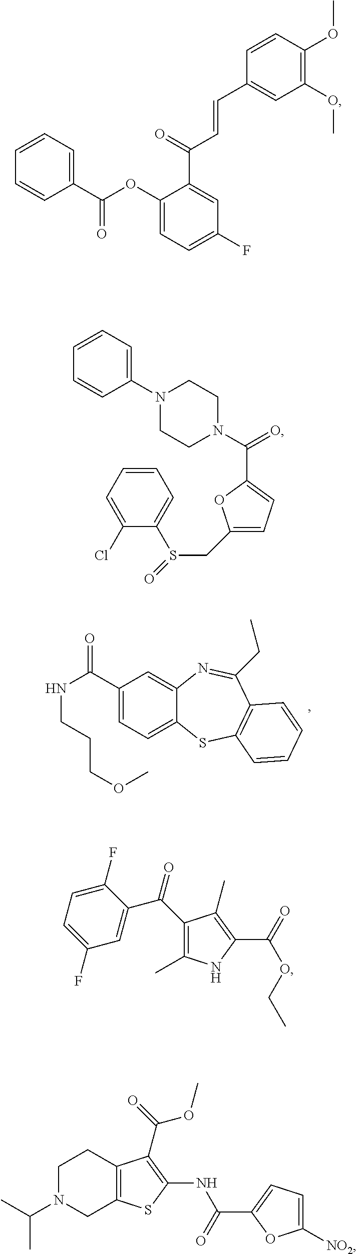

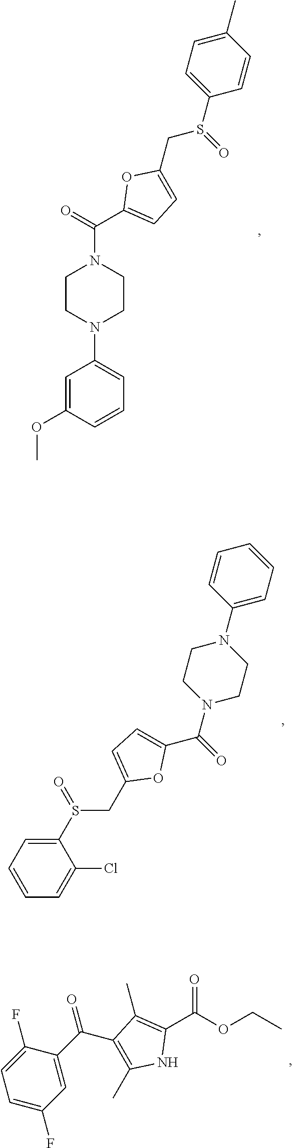 [SMILES: COc1cccc(N2CCN(C(=O)c3ccc(CS(=O)c4ccc(C)cc4)o3)CC2)c1 ; O=C(c1ccc(CS(=O)c2ccccc2Cl)o1)N1CCN(c2ccccc2)CC1 ; CCOC(=O)c1[nH]c(C)c(C(=O)c2cc(F)ccc2F)c1C]

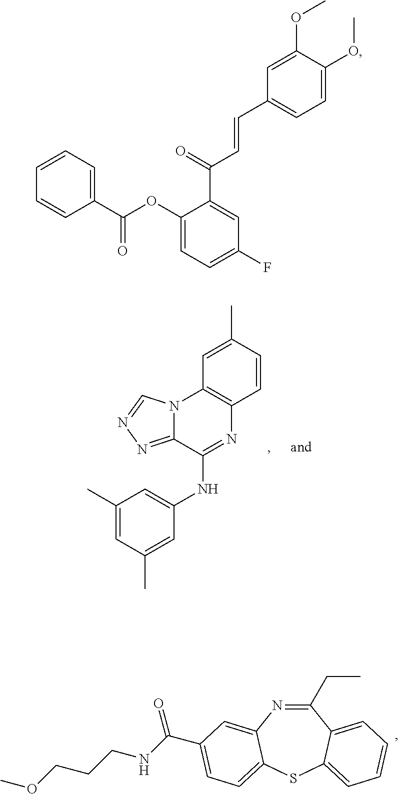

or a pharmaceutically acceptable salt of any of the foregoing.

Some embodiments provide a method for treating an α-synuclein associated disease or disorder in a subject in need thereof, comprising administering to the subject a therapeutically effective amount of a compound of selected from the group consisting of:

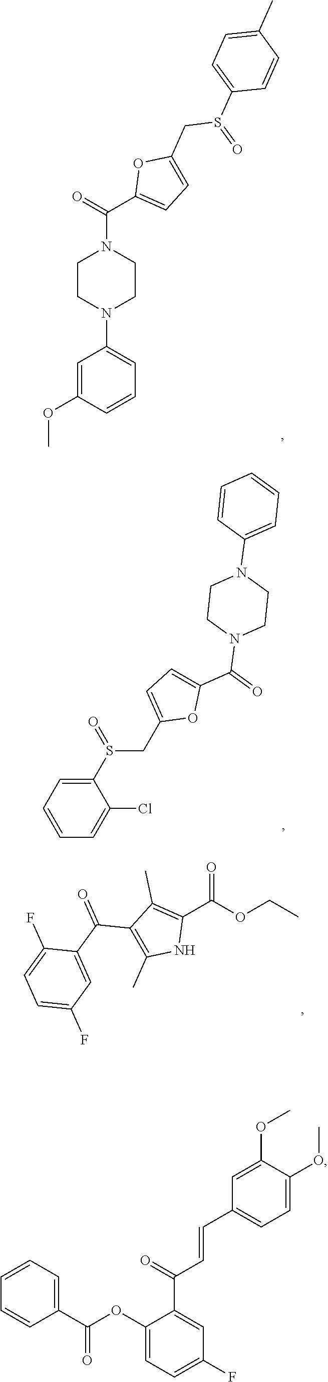 [SMILES: COc1cccc(N2CCN(C(=O)c3ccc(CS(=O)c4ccc(C)cc4)o3)CC2)c1 ; O=C(c1ccc(CS(=O)c2ccccc2Cl)o1)N1CCN(c2ccccc2)CC1 ; CCOC(=O)c1[nH]c(C)c(C(=O)c2cc(F)ccc2F)c1C ; COc1ccc(/C=C/C(=O)c2cc(F)ccc2OC(=O)c2ccccc2)cc1OC]

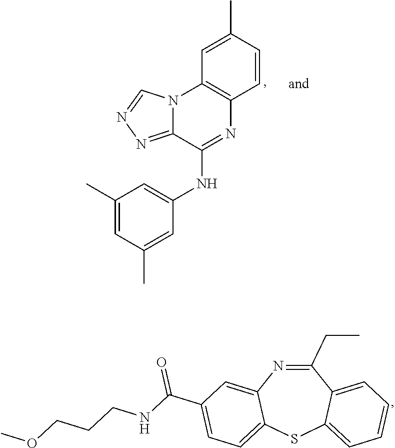 [SMILES: Cc1cc(C)cc(Nc2nc3ccc(C)cc3n3cnnc23)c1 ; CCC1=Nc2cc(C(=O)NCCCOC)ccc2Sc2ccccc21]

or a pharmaceutically acceptable salt of any of the foregoing.

Also provided herein is a method for treating Parkinson's disease in a subject in need thereof, comprising administering to the subject a therapeutically effective amount of any one of Compounds 1-16, or a pharmaceutically acceptable salt thereof, or a pharmaceutical composition as provided herein.

Also provided herein is a method for treating Parkinson's disease in a subject in need thereof, comprising administering to the subject a therapeutically effective amount of any one of Compounds 1-10, or a pharmaceutically acceptable salt thereof, or a pharmaceutical composition as provided herein.

Also provided herein is a method for treating dementia with Lewy bodies in a subject in need thereof, comprising administering to the subject a therapeutically effective amount of any one of Compounds 1-16, or a pharmaceutically acceptable salt thereof, or a pharmaceutical composition as provided herein.

Also provided herein is a method for treating dementia with Lewy bodies in a subject in need thereof, comprising administering to the subject a therapeutically effective amount of any one of Compounds 1-10, or a pharmaceutically acceptable salt thereof, or a pharmaceutical composition as provided herein.

Also provided herein is a method for treating multiple systems atrophy in a subject in need thereof, comprising administering to the subject a therapeutically effective amount of any one of Compounds 1-16, or a pharmaceutically acceptable salt thereof, or a pharmaceutical composition as provided herein.

Also provided herein is a method for treating multiple systems atrophy in a subject in need thereof, comprising administering to the subject a therapeutically effective amount of any one of Compounds 1-10, or a pharmaceutically acceptable salt thereof, or a pharmaceutical composition as provided herein.

Also provided herein is a method for treating prodromal REM sleep behavior disorder (RBD) in a subject in need thereof, comprising administering to the subject a therapeutically effective amount of any one of Compounds 1-16, or a pharmaceutically acceptable salt thereof, or a pharmaceutical composition as provided herein.

Also provided herein is a method for treating prodromal REM sleep behavior disorder (RBD) in a subject in need thereof, comprising administering to the subject a therapeutically effective amount of any one of Compounds 1-10, or a pharmaceutically acceptable salt thereof, or a pharmaceutical composition as provided herein.

Provided herein is a method of treating an α-synuclein associated disease or disorder in a subject, comprising administering to a subject identified or diagnosed as having an α-synuclein associated disease or disorder a therapeutically effective amount of any one of Compounds 1-16, or a pharmaceutically acceptable salt thereof, or a pharmaceutical composition as provided herein.

Provided herein is a method of treating an α-synuclein associated disease or disorder in a subject, comprising administering to a subject identified or diagnosed as having an α-synuclein associated disease or disorder a therapeutically effective amount of any one of Compounds 1-10, or a pharmaceutically acceptable salt thereof, or a pharmaceutical composition as provided herein.

This disclosure also provides a method of treating an α-synuclein associated disease or disorder in a subject, comprising: determining that the disease or disorder in the subject is an α-synuclein associated disease or disorder; and administering to the subject a therapeutically effective amount of any one of Compounds 1-16, or a pharmaceutically acceptable salt thereof, or a pharmaceutical composition as provided herein.

This disclosure also provides a method of treating an α-synuclein associated disease or disorder in a subject, comprising: determining that the disease or disorder in the subject is an α-synuclein associated disease or disorder; and administering to the subject a therapeutically effective amount of any one of Compounds 1-10, or a pharmaceutically acceptable salt thereof, or a pharmaceutical composition as provided herein.

Provided herein is a method of treating a subject, comprising administering a therapeutically effective amount of any one of Compounds 1-16, or a pharmaceutically acceptable salt thereof, or a pharmaceutical composition as provided herein, to a subject having a clinical record that indicates that the subject has an α-synuclein associated disease or disorder.

Provided herein is a method of treating a subject, comprising administering a therapeutically effective amount of any one of Compounds 1-10, or a pharmaceutically acceptable salt thereof, or a pharmaceutical composition as provided herein, to a subject having a clinical record that indicates that the subject has an α-synuclein associated disease or disorder.

This disclosure also provides a method for inhibiting α-synuclein translation in a mammalian cell, comprising contacting the mammalian cell with an effective amount of any one of Compounds 1-16, or a pharmaceutically acceptable salt thereof.

This disclosure also provides a method for inhibiting α-synuclein translation in a mammalian cell, comprising contacting the mammalian cell with an effective amount of any one of Compounds 1-10, or a pharmaceutically acceptable salt thereof.

This disclosure further provides a method for inhibiting Lewy body formation in mammalian brain tissue, comprising contacting the mammalian brain tissue with an effective amount of any one of Compounds 1-16, or a pharmaceutically acceptable salt thereof.

This disclosure further provides a method for inhibiting Lewy body formation in mammalian brain tissue, comprising contacting the mammalian brain tissue with an effective amount of any one of Compounds 1-10, or a pharmaceutically acceptable salt thereof.

Also provided herein is a pharmaceutical composition comprising any one of Compounds 1-16, or a pharmaceutically acceptable salt thereof, and one or more pharmaceutically acceptable excipients.

Also provided herein is a pharmaceutical composition comprising any one of Compounds 1-10, or a pharmaceutically acceptable salt thereof, and one or more pharmaceutically acceptable excipients.

Also provided herein is a method for detecting an α-synuclein associated disease or disorder in a subject in need thereof, comprising administering to the subject an effective amount of any one of Compounds 1-16, or a pharmaceutically acceptable salt of any of the foregoing; wherein the compound, or a pharmaceutically acceptable salt thereof, is radiolabeled.

Also provided herein is a method for detecting an α-synuclein associated disease or disorder in a subject in need thereof, comprising administering to the subject an effective amount of any one of Compounds 1-10, or a pharmaceutically acceptable salt of any of the foregoing; wherein the compound, or a pharmaceutically acceptable salt thereof, is radiolabeled.

Also provided herein is a method for detecting Parkinson's disease in a subject in need thereof, comprising administering to the subject an effective amount of an radiolabeled compound described herein, or a pharmaceutically acceptable salt thereof.

Also provided herein is a method for detecting dementia with Lewy bodies in a subject in need thereof, comprising administering to the subject an effective amount of an radiolabeled compound described herein, or a pharmaceutically acceptable salt thereof.

Also provided herein is a method for detecting multiple systems atrophy in a subject in need thereof, comprising administering to the subject an effective amount of an radiolabeled compound described herein, or a pharmaceutically acceptable salt thereof.

Also provided herein is a method for detecting prodromal REM sleep behavior disorder (RBD) in a subject in need thereof, comprising administering to the subject an effective amount of an radiolabeled compound described herein, or a pharmaceutically acceptable salt thereof.

Provided herein is a method of detecting an α-synuclein associated disease or disorder in a subject, comprising administering to a subject suspected as having an α-synuclein associated disease or disorder an effective amount of an radiolabeled compound described herein, or a pharmaceutically acceptable salt thereof.

This disclosure also provides a method for detecting α-synuclein translation in a mammalian cell, comprising contacting the mammalian cell with an effective amount of an radiolabeled compound described herein, or a pharmaceutically acceptable salt thereof.

This disclosure further provides a method for detecting Lewy body formation in mammalian brain tissue, comprising contacting the mammalian brain tissue with an effective amount of an radiolabeled compound described herein, or a pharmaceutically acceptable salt thereof.

Also provided herein is an imaging composition comprising an radiolabeled compound described herein, or a pharmaceutically acceptable salt thereof, and one or more pharmaceutically acceptable excipients.

Other embodiments include those described in the Detailed Description and/or in the claims.

### BRIEF DESCRIPTION OF THE FIGURES

FIG. 1 illustrates the concentration-dependent activities of compound 10 in the target and counterscreen assays. Activity curves for compound 10: the alpha-synuclein IRE containing H4 neuroglioblastoma primary screening cell line (A), the H4 neuroglioblastoma cell line with the IRE stem-loop removed from the 5′UTR of alpha-synuclein (B), the H4 neuroglioblastoma cells containing the prion protein IRE containing in the 5′UTR upstream of luciferase (C). For the protocol, see Ross, et al., Probes Report, Molecular Libraries (Pathways to Discovery) “Identification of a small molecule that selectively inhibits alpha-synuclein translational expression” (2011).

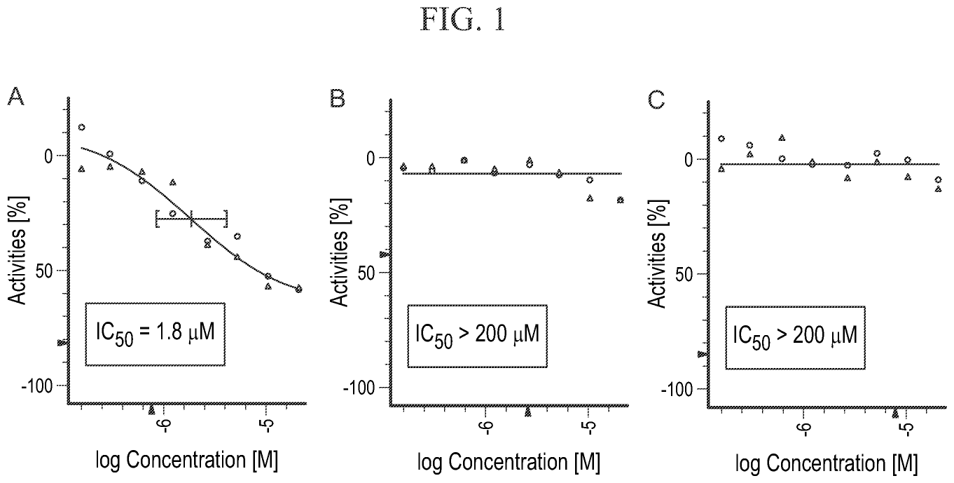

FIG. 2 illustrates the results of ELISA experiments with compound 10 in SH-SY5Y cells.

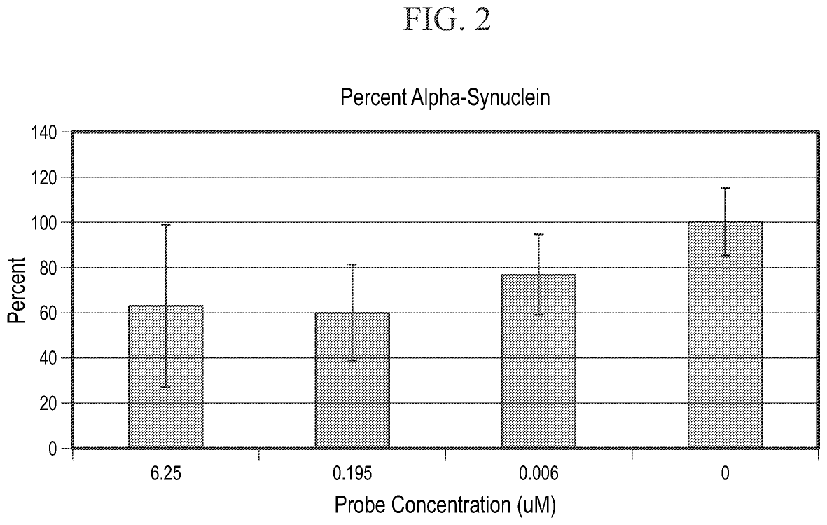

FIG. 3 illustrates the Western blot results from compound 10 in H4 cells.

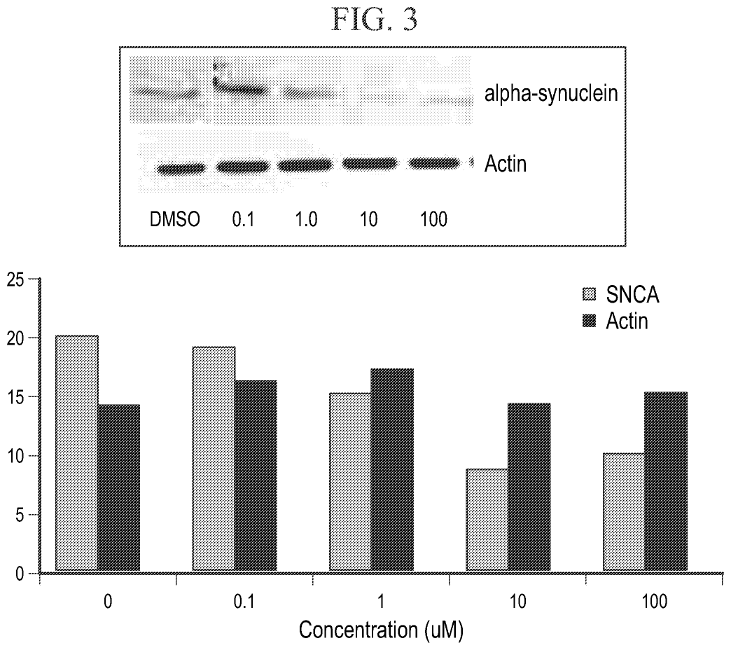

FIG. 4 illustrates Western blots showing α-syn levels in ipSC derived DA neurons after treatment with posiphen and compounds 10, 2, 3, or 9 reduced (N=3, β-actin standardized, Top: 10 μM compound, Bottom: 1 μM compound). Also shown are β-actin standardized Western blots showing the relative potencies of selected compounds described herein to posiphen at lowering α-Synuclein protein expression in iPSC derived DA Neuronal lysates. Induced pluripotent stem cells (IpSC) from a control patient were differentiated by the method of Kim et al (2021) into DA neurons and allowed to differentiate for 6 weeks. Posiphen, Compound 10, Compound 2, Compound 3, and Compound 9 (high dose, 10 μM, top panel and middle dose, 1 uM bottom panel) were added for 72 h.

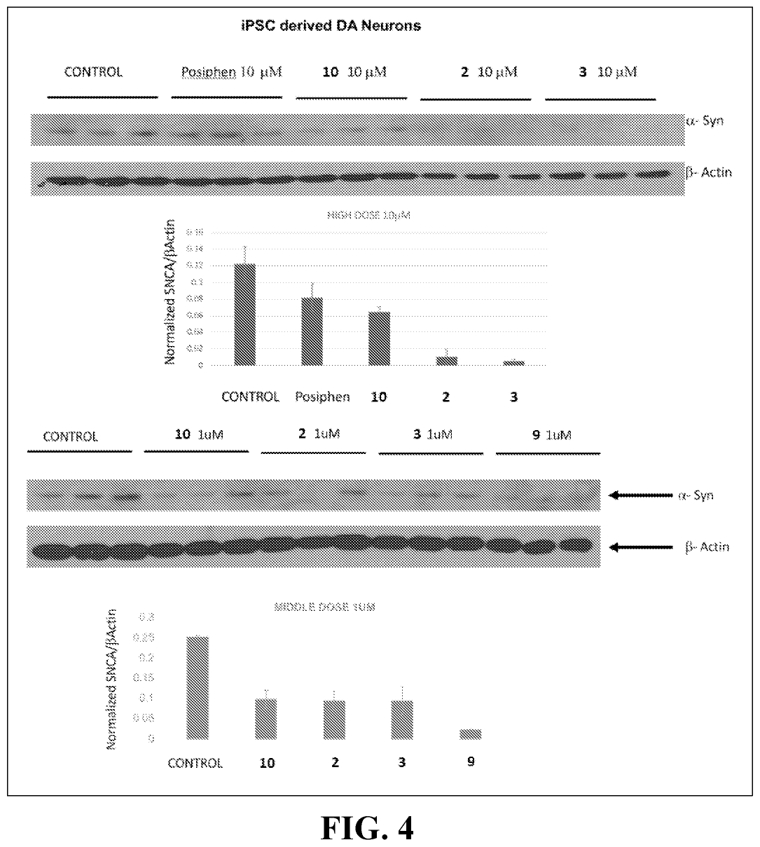

FIG. 5 illustrates PET imaging of timing/uptake of [¹¹C]-labeled compound 9 (Compound 9-r) in mouse brains subregions. After injection with a bolus of 9-r, mice were analyzed with a PET scanner. The time-course was quantitated, brain retention, distribution of 9-r into midbrain subregions.

FIG. 6 illustrates PET imaging of timing/uptake of [¹¹C]-labeled compound 3 (Compound 3-r) in mouse brains subregions. After injection with a bolus of 3-r, mice were analyzed with a PET scanner. The time-course was quantitated, brain retention, distribution of 3-r into midbrain subregions.

FIG. 7 illustrates PET imaging of timing/uptake of [¹¹C]-labeled compound 2 (Compound 2-r) in mouse brains subregions. After injection with a bolus of 2-r, mice were analyzed with a PET scanner. The time-course was quantitated, brain retention, distribution of 2-r into midbrain subregions.

FIG. 8 illustrates the concentration-dependent activities of compound 2 in the target and counter screen assays. Activity curves for compound 2: the alpha-synuclein IRE containing H4 neuroglioblastoma primary screening cell line (A, B), the H4 neuroglioblastoma cells containing the prion protein IRE containing in the 5′UTR upstream of luciferase (C), the H4 neuroglioblastoma cell line with the IRE stem-loop removed from the 5′UTR of alpha-synuclein (D). For the protocol, see Ross, et al., Probes Report, Molecular Libraries (Pathways to Discovery) “Identification of a small molecule that selectively inhibits alpha-synuclein translational expression” (2011).

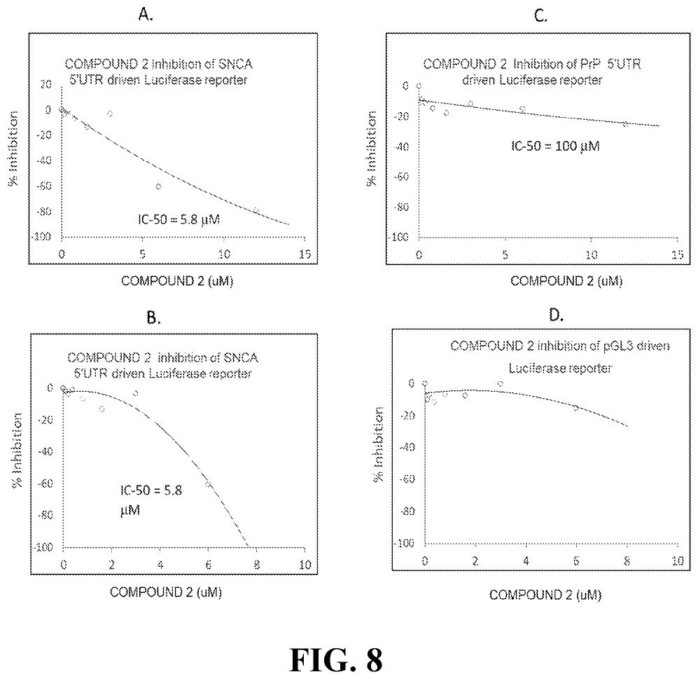

FIG. 9 illustrates that Compound 10, Compound 2, Compound 3, and Compound 9 selectively inhibit α-Synuclein protein expression in control iPSC patient derived dopaminergic neurons. Induced pluripotent stem cells (IpSC) from a control patient were differentiated by the method of Kim et al (2021) into DA neurons and allowed to differentiate for 6 weeks. Compound 10, Compound 2, Compound 3, and Compound 9 (1 μM) were added for 72 h. Cells were harvested, lysed and western blots (panel A) performed for the detection of α-Syn, β-Syn, γ-Syn, Ferritin, APP and Prion proteins and normalized to β-Actin (panel B).

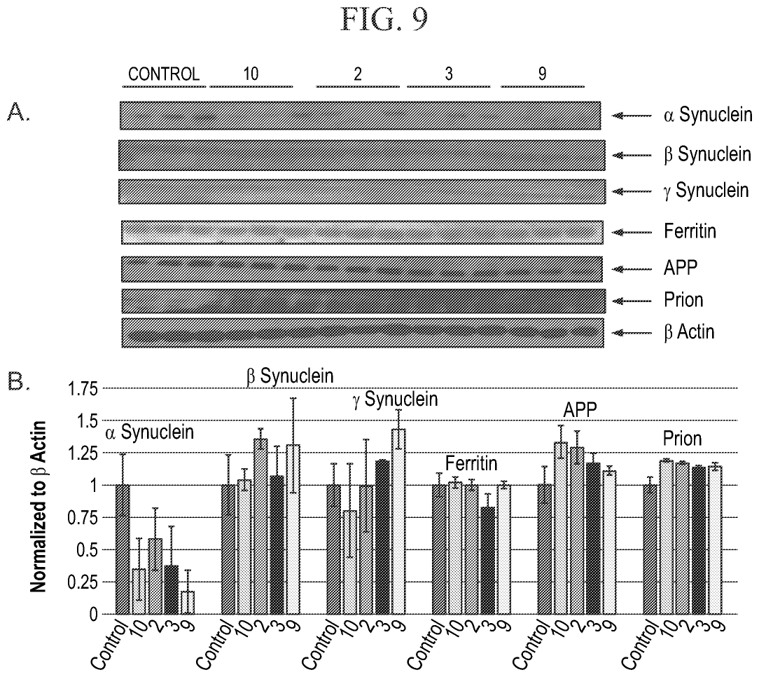

FIG. 10 illustrates α-syn levels measured by ELISA (Invitrogen) in normal patient ipSC derived DA neurons lysates after treatment with posiphen (10 μM) and compound 10 (10, 1, 0.1 μM) (N=3). Induced pluripotent stem cells (IpSC) from a control patient were differentiated by the method of Kim et al (2021) into DA neurons and allowed to differentiate for 6 weeks. Posiphen and Compound 10, were added for 72 h.

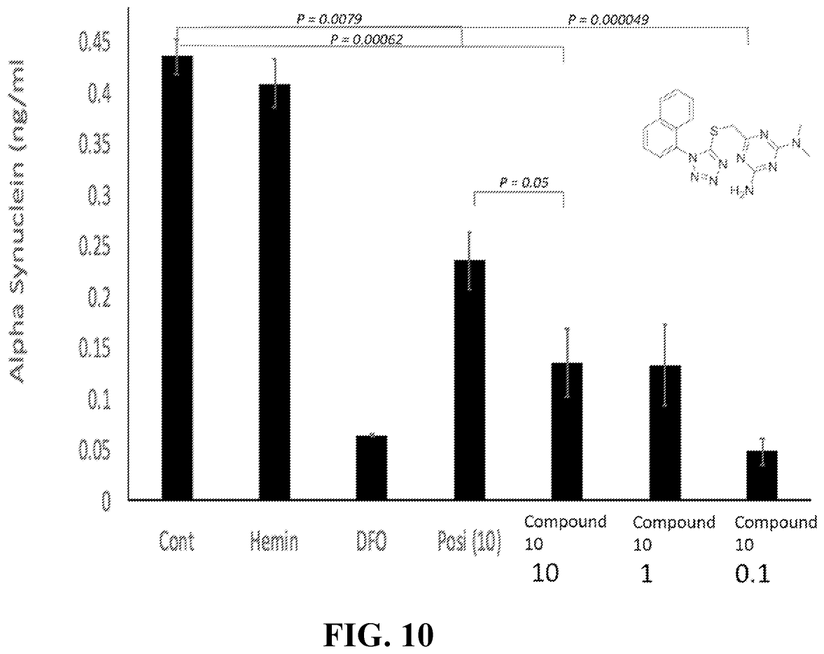

FIG. 11 illustrates α-syn levels measured by ELISA (Invitrogen) in normal patient ipSC derived DA neuron lysates after treatment with posiphen and compounds 10, 2, 3, or 9 (N=3). Induced pluripotent stem cells (IpSC) from a control patient were differentiated by the method of Kim et al (2021) into DA neurons and allowed to differentiate for 6 weeks. Compound 10, Compound 2, Compound 3, and Compound 9 (panel A: low dose, (0.1 uM); panel B: middle dose (1 uM), and panel C high dose including Posiphen for comparison 10 μM) were added for 72 h. Hemin and Deferiprone were added in separate wells (panel D). Cells were harvested and lysed and measured for α-Syn.

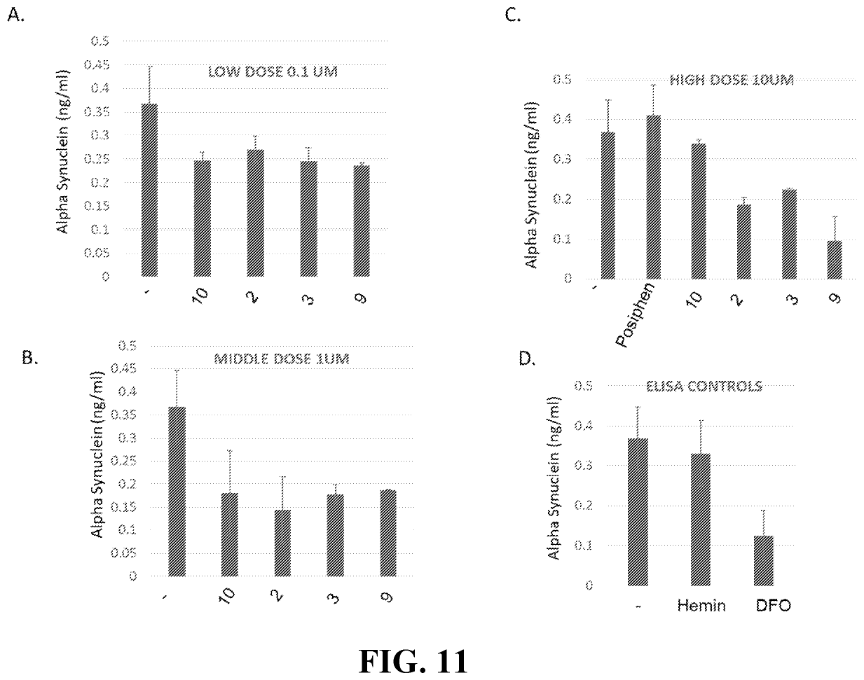

FIG. 12 illustrates the potency of Compound 10, Compound 2, and Compound 3, relative to posiphen in lowering α-Syn expression in Triple SNCA Cortical Neurons. Triple SNCA cortical neurons (Khurana Lab, BWH, Boston) were grown to confluence and treated for 72 h in triplicate with posiphen and Compound 10, Compound 2, and Compound 3 at doses of 0.1, 1 and 10 μM. Cells were harvested, lysed and blotted for α-Syn protein (upper panel) and normalized to β-Actin (lower panel).

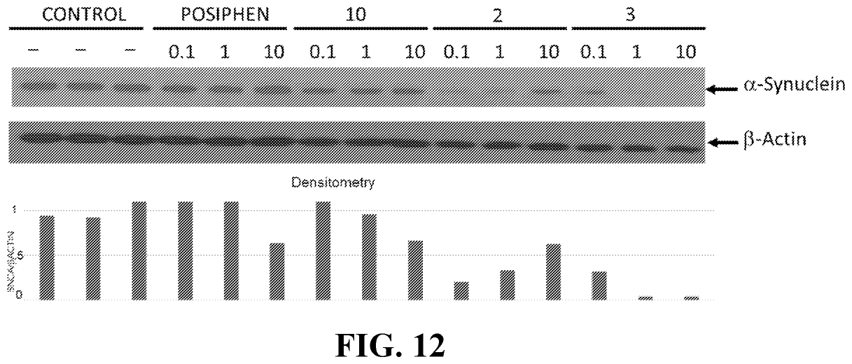

FIG. 13 illustrates the potency of Compound 10, Compound 2, and Compound 3, relative to posiphen at lowering α-Syn expression in Triple SNCA Cortical Neurons. Triple SNCA cortical neurons (Khurana Lab, BWH, Boston) were grown to confluence and treated for 72 h in triplicate with posiphen and Compound 10, Compound 2, Compound 3 at doses of 0.1, 1 and 10 μM. Cells were harvested, lysed and lysates measured for α-Syn by ELISA.

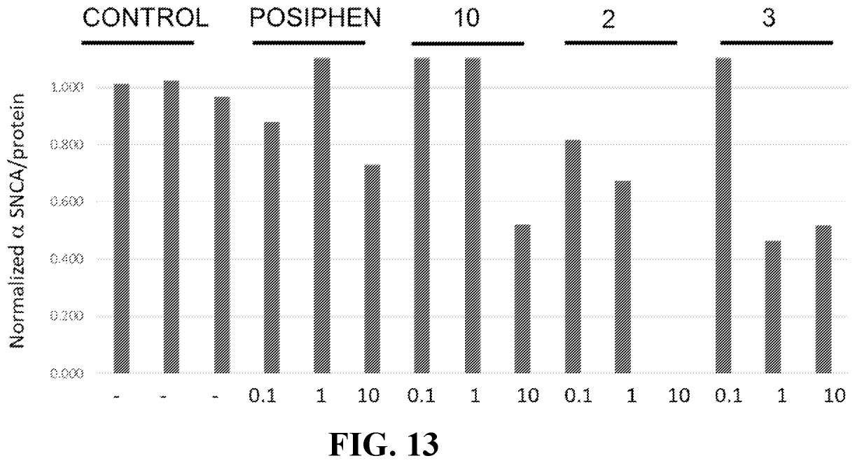

FIG. 14 illustrates that immunocytochemical quantitation of effect of Compound 2 on α-Syn expression in tyrosine hydroxylase positive (TH+) α-synuclein positive (SNCA+) neurons. Panel A: antibody stains for TH and α-Syn. TH positive cells were counted. TH positive and SNCA positive cells were counted. The quantification was done by collecting 9 image fields/well with 3 wells/condition Panel B. The images were fed into Cell Profiler (Broad software). At low dose Compound 2 significantly decreased the number of TH/SNCA positive cells from 30 to 18%.

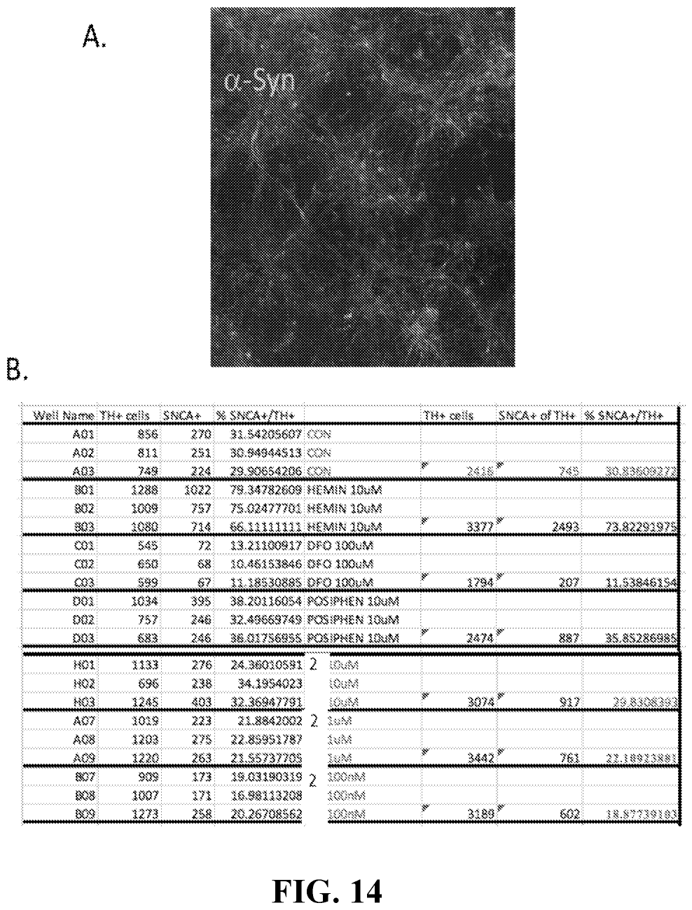

FIG. 15 illustrates that spinach fluorescent assay showing compound 11 binds strongly to the SNCA IRE (Karen Wu, Lucerna NY). SNCA IRE fluorescence sensor R7 (panel A) detected strong fluorescence when bound by an anti-sense oligonucleotide (ASO-3) and also significant increases in binding by addition of Compound 2 and Compound 11 (panel C). Compound 2 and Compound 11 are likely SNCA IRE intercalators. Posiphen and synuleozoid (panel B) have no measurable effect or quenching effect toward sensor's fluorescence.

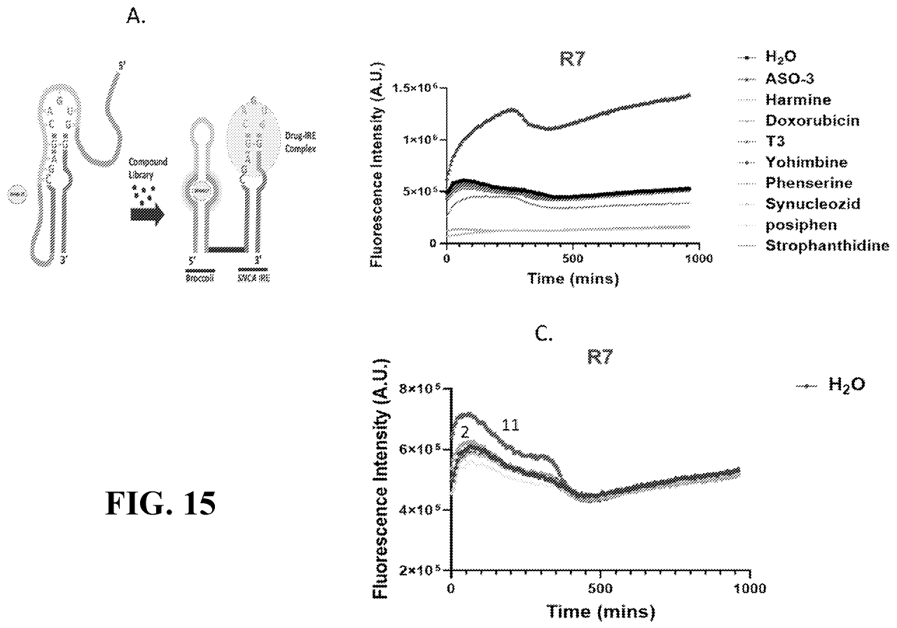

FIG. 16 illustrates the concentration-dependent activities of compound 3 in the target and counter screen assays. Activity curves for compound 3: the alpha-synuclein IRE containing H4 neuroglioblastoma primary screening cell line (orange); the H4 neuroglioblastoma cells containing the prion protein IRE containing in the 5′UTR upstream of luciferase (yellow), the H4 neuroglioblastoma cell line with the IRE stem-loop removed from the 5′UTR of alpha-synuclein (Green). For the protocol, see Ross, et al., Probes Report, Molecular Libraries (Pathways to Discovery) “Identification of a small molecule that selectively inhibits alpha-synuclein translational expression” (2011).

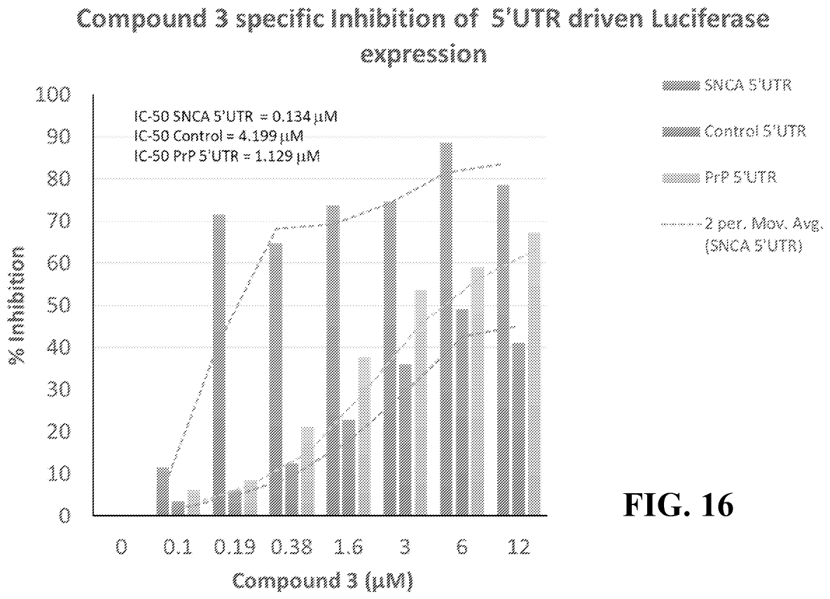

FIG. 17 illustrates western analysis of alpha Synuclein in SHSY5 differentiated (standard PMA+RA protocol) dopaminergic (DA) neurons lysates treated with Compound 10 for 72 h at the indicated doses (panel A) normalized to beta actin control. Panel B shows alpha Synuclein ELISA (Invitrogen) measurements from SHSY5 undifferentiated neurons treated with the indicated doses of compound 10 (2 separate assays) for 48 h.

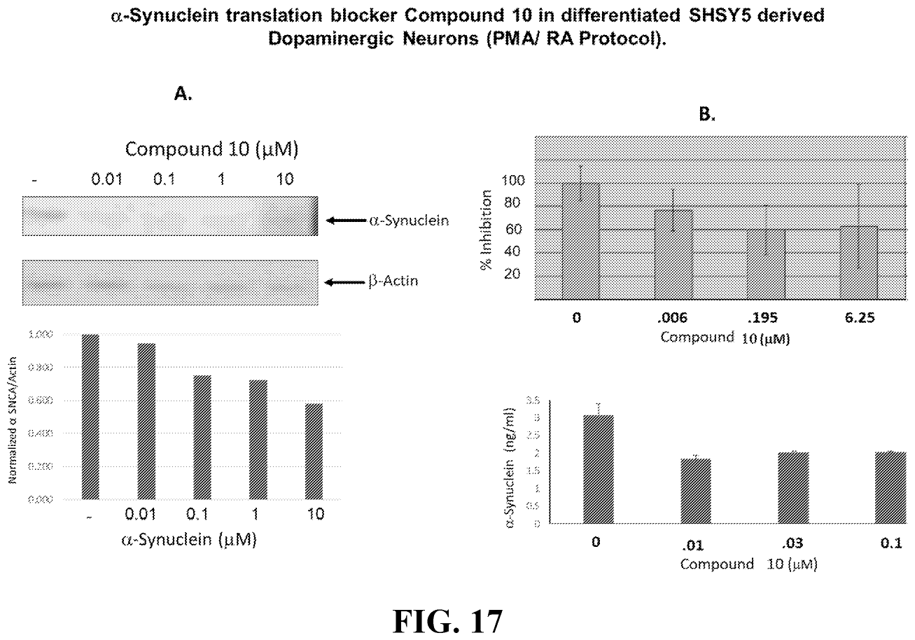

FIG. 18 illustrates alpha Synuclein ELISA (Invitrogen) measurements from SHSY5 undifferentiated neurons treated with the indicated doses (triplicate wells) of compounds 2, 13, 11, 14, 15, and 16) for 48 h.

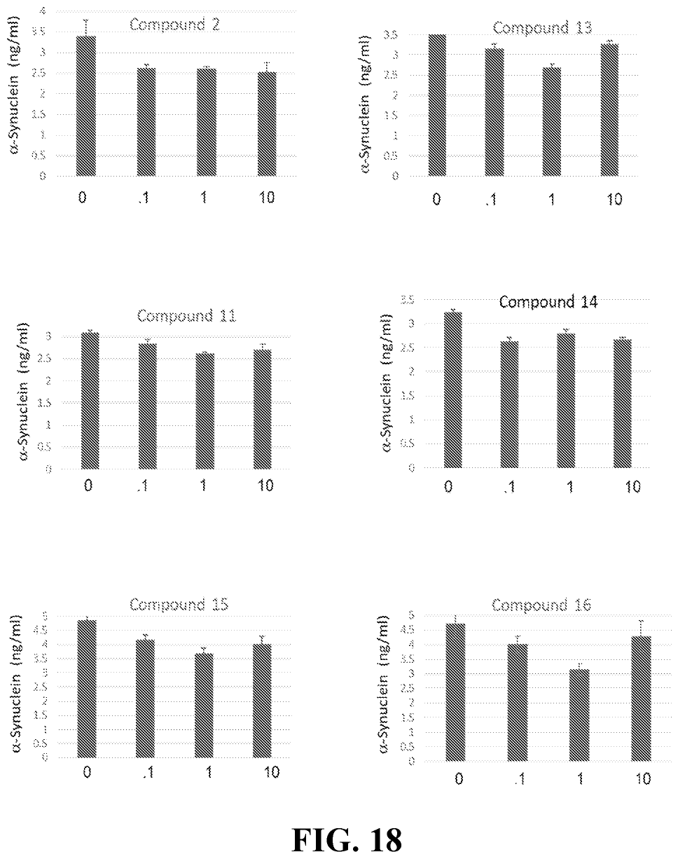

### DETAILED DESCRIPTION

This disclosure provides compounds, and pharmaceutically acceptable salts thereof, that can inhibit the 5′ UTR of α-synuclein mRNA. These chemical entities are useful, e.g., for treating a condition, disease or disorder in which α-synuclein contributes to the pathology and/or symptoms and/or progression of the condition, disease or disorder (e.g., Parkinson's disease) in a subject (e.g., a human). This disclosure also provides compositions containing the same as well as methods of using and making the same.

Alpha-synuclein is an approximately 15 KDa protein implicated in the pathogenesis of neurodegenerative alpha-synucleinopathies, including Parkinson's disease (PD), the most prevalent movement disorder in humans. In these disorders, alpha-synuclein undergoes a conformational change and subsequent oligomerization, which causes a toxic gain of function, neurodegeneration, and deposition of alpha-synuclein aggregates in the form of Lewy bodies. Increased alpha-synuclein levels caused by gene duplication causes familial PD, and GATA transcription factors (3) directly regulate the PD-linked alpha-synuclein gene. In this regard, regulation of alpha-synuclein translation is physiologically relevant to Lewy body dementia brains, which exhibit lowered alpha-synuclein mRNA but higher insoluble protein. This suggests misregulation of alpha-synuclein translation in addition to protein clearance by chaperones. Consequently, our therapeutic strategy is to limit alpha-synuclein translation.

Alpha-synuclein translation is governed, at least in part, by an iron-responsive element (IRE) in the 5′ untranslated region (5′-UTR) stem-loop of alpha-synuclein mRNA. Iron-responsive elements (IREs) are RNA stem loops in the UTRs of ferritin and transferring-receptor mRNAs that are critical to iron homeostasis. However, the protein binding profile of the alpha-synuclein IRE is different from the canonical IREs of iron homeostasis. These findings reflect the sensitivity of this RNA sequence as a target for therapeutic intervention.

### Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

As used herein, the phrase “Compounds 1-16”, and pharmaceutically acceptable salts thereof, refer to the two sets of compounds shown below.

As used herein, the phrase “Compounds 1-10”, and pharmaceutically acceptable salts thereof, refer to the compounds shown below.

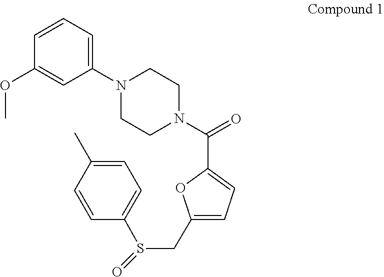 [SMILES: COc1cccc(N2CCN(C(=O)c3ccc(CS(=O)c4ccc(C)cc4)o3)CC2)c1]

[(4-(3-methoxyphenyl)piperazin-1-yl)(5-((p-tolylsulfinyl)methyl)furan-2-yl)methanone]

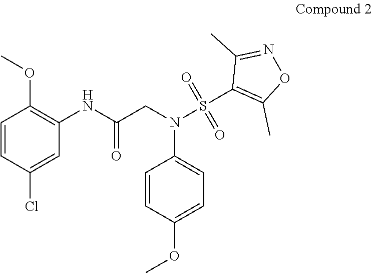 [SMILES: COc1ccc(N(CC(=O)Nc2cc(Cl)ccc2OC)S(=O)(=O)c2c(C)noc2C)cc1]

[N-(5-chloro-2-methoxyphenyl)-2-((N-(4-methoxyphenyl)-3,5-dimethylisoxazole)-4-sulfonamido)acetamide]

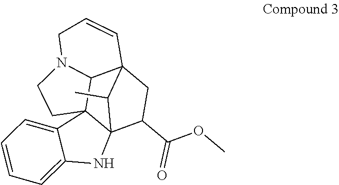

[methyl 14-methyl-4,5,11,12-tetrahydro-1H,3a¹H,6H-3a,5a-methanoindolizino[8,1-cd]carbazole-5-carboxylate]

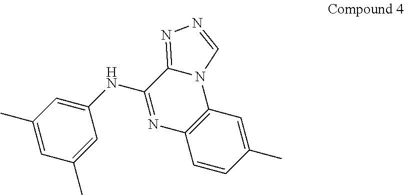

[N-(3,5-dimethylphenyl)-8-methyl-[1,2,4]triazolo[4,3-a]quinoxalin-4-amine]

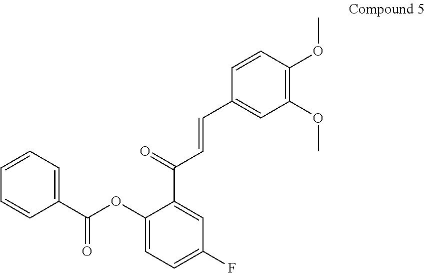 [SMILES: COc1ccc(/C=C/C(=O)c2cc(F)ccc2OC(=O)c2ccccc2)cc1OC]

[(E)-2-(3-(3,4-dimethoxyphenyl)acryloyl)-4-fluorophenyl benzoate]

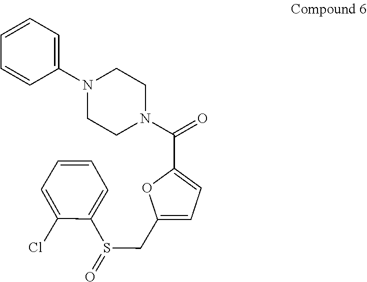 [SMILES: O=C(c1ccc(CS(=O)c2ccccc2Cl)o1)N1CCN(c2ccccc2)CC1]

[(5-(((2-chlorophenyl)sulfinyl)methyl)furan-2-yl)(4-phenylpiperazin-1-yl)methanone]

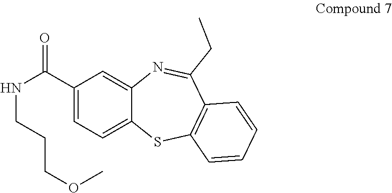 [SMILES: CCC1=Nc2cc(C(=O)NCCCOC)ccc2Sc2ccccc21]

[11-ethyl-N-(3-methoxypropyl)dibenzo[b,f][1,4]thiazepine-8-carboxamide]

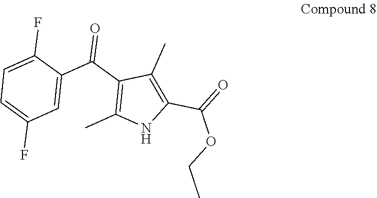 [SMILES: CCOC(=O)c1[nH]c(C)c(C(=O)c2cc(F)ccc2F)c1C]

[ethyl 4-(2,5-difluorobenzoyl)-3,5-dimethyl-1H-pyrrole-2-carboxylate]

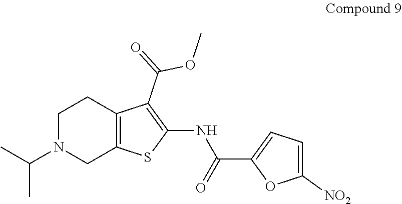 [SMILES: COC(=O)c1c(NC(=O)c2ccc([N+](=O)[O-])o2)sc2c1CCN(C(C)C)C2]

[methyl 6-isopropyl-2-(5-nitrofuran-2-carboxamido)-4,5,6,7-tetrahydrothieno[2,3-c]pyridine-3-carboxylate]

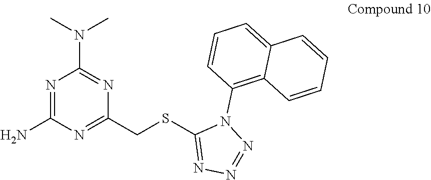 [SMILES: CN(C)c1nc(N)nc(CSc2nnnn2-c2cccc3ccccc23)n1]

[N²,N²-dimethyl-6-(((1-(naphthalen-1-yl)-1H-tetrazol-5-yl)thio)methyl)-1,3,5-triazine-2,4-diamine].

As used herein, the phrase “Compounds 11-16”, and pharmaceutically acceptable salts thereof, refer to the compounds shown below.

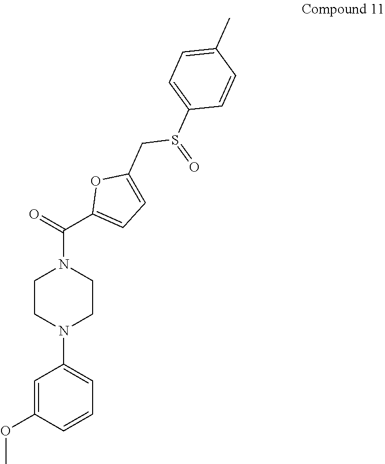 [SMILES: COc1cccc(N2CCN(C(=O)c3ccc(CS(=O)c4ccc(C)cc4)o3)CC2)c1]

1-(3-Methoxyphenyl)-4-(5-{[(4-methylphenyl)sulfinyl]methyl}-2-furoyl)piperazine.

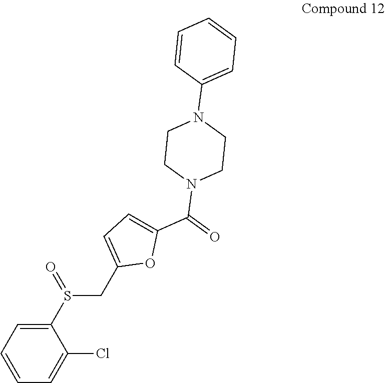 [SMILES: O=C(c1ccc(CS(=O)c2ccccc2Cl)o1)N1CCN(c2ccccc2)CC1]

1-(5-{[(2-Chlorophenyl)sulfinyl]methyl}-2-furoyl)-4-phenylpiperazine.

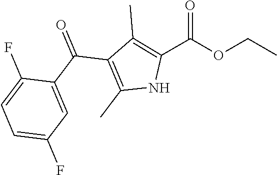 [SMILES: CCOC(=O)c1[nH]c(C)c(C(=O)c2cc(F)ccc2F)c1C]

Ethyl 4-(2,5-difluorobenzoyl)-3,5-dimethyl-1H-pyrrole-2-carboxylate.

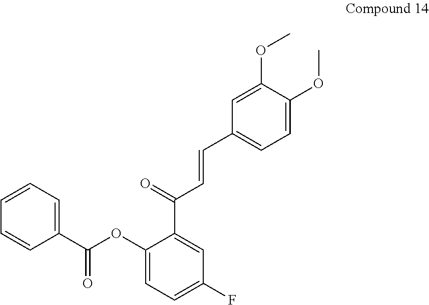 [SMILES: COc1ccc(/C=C/C(=O)c2cc(F)ccc2OC(=O)c2ccccc2)cc1OC]

2-[(2E)-3-(3,4-dimethoxyphenyl)prop-2-enoyl]-4-fluorophenyl benzoate.

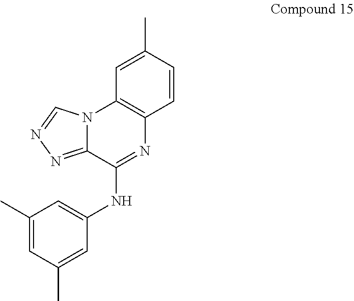 [SMILES: Cc1cc(C)cc(Nc2nc3ccc(C)cc3n3cnnc23)c1]

N-(3,5-dimethylphenyl)-8-methyl[1,2,4]triazolo[4,3-a]quinoxalin-4-amine.

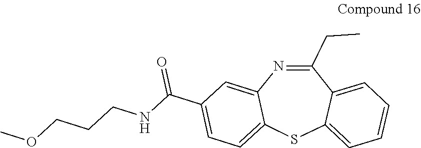 [SMILES: CCC1=Nc2cc(C(=O)NCCCOC)ccc2Sc2ccccc21]

11-ethyl-N˜8˜-(3-methoxypropyl)dibenzo[b,f][1,4]thiazepine-8-carboxamide.

The phrase “the compounds described herein” refer to any single compound or combination of compounds of Compound 1-16, and pharmaceutically acceptable salts thereof, for example, Compounds 1-16, Compounds 1-15, Compounds 1-14, Compounds 1-13, Compounds 1-12, Compounds 1-11, Compounds 1-10, Compounds 1-9, Compounds 1-8, Compounds 1-7, Compounds 1-6, Compounds 1-5, Compounds 1-4, Compounds 1-3, Compounds 1-2, and the like.

The term “about” when referring to a number or a numerical range means that the number or numerical range referred to is an approximation, for example, within experimental variability and/or statistical experimental error, and thus the number or numerical range may vary up to +10% of the stated number or numerical range.

The term “acceptable” with respect to a formulation, composition or ingredient, as used herein, means having no persistent detrimental effect on the general health of the subject being treated.

The term “inhibit” or “inhibition of” means to reduce by a measurable amount, or to prevent entirely (e.g., 100% inhibition) relative to a baseline or starting level.

The term “activate” or “activation of” means to increase by a measurable amount, e.g., 50% activation refers to an increase of 50% relative to a baseline or starting level.

The terms “effective amount” or “therapeutically effective amount,” as used herein, refer to a sufficient amount of a chemical entity being administered which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study. An “effective amount” also refers to an amount sufficient to achieve a desired effect, for example, in the context of medical imaging, an effective amount is sufficient to provide a sufficient signal under imaging conditions (such as PET or MRI scanning) to produce an image.

The term “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutically acceptable salt” refers to a formulation of a compound that does not cause significant irritation to an organism to which it is administered and does not abrogate the biological activity and properties of the compound. Included among such salts are the following: acetate, adipate, alginate, aspartate, benzoate, benzenesulfonate, bisulfate butyrate, citrate, camphorate, camphorsulfonate, cyclopentanepropionate, digluconate, dodecylsulfate, ethanesulfonate, fumarate, glucoheptanoate, glycerophosphate, hemis sulfate heptanoate, hexanoate, hydrochloride, hydrobromide, hydroiodide, 2-hydroxyethanesulfonate, lactate, maleate, methanesulfonate, 2-naphthalensulfonate, nicotinate, oxalate, pamoate, pectinate, propionate, succinate, tartrate, thiocyanate, tosylate and undecanoate. Base salts include ammonium salts, alkali metal salts such as sodium and potassium salts, alkaline earth metal salts such as calcium and magnesium salts, salt with organic bases such as dicyclohexylamine salts, N-methyl-D-glucamine, and salts with amino acids such as arginine, lysine, and so forth. Also, the basic nitrogen-containing groups can be quarternized with such agents as lower alkyl halides, such as methyl, ethyl, propyl, and butyl chloride, bromides and iodides; dialkyl sulfates like dimethyl, diethyl, dibutyl and diamyl sulfates, long chain halides such as decyl, lauryl, myristyl and stearyl chlorides, bromides and iodides, aralkyl halides like benzyl and phenethyl bromides and others. Water or oil-soluble or dispersible products are thereby obtained.

The term “pharmaceutical composition” refers to a mixture of a compound, or a pharmaceutically acceptable salt thereof, described herein with other chemical components (referred to collectively herein as “pharmaceutically acceptable excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: oral, intravenous, aerosol, parenteral, ophthalmic, pulmonary, and topical administration.

The term “imaging composition” refers to a mixture of a radiolabeled compound, or a pharmaceutically acceptable salt thereof, described herein with other chemical components (referred to collectively herein as “pharmaceutically acceptable excipients”), such as carriers, stabilizers, diluents, dispersing agents, suspending agents, and/or thickening agents. The imaging composition facilitates administration of the compound to an organism. Multiple techniques of administering a compound exist in the art including, but not limited to: oral, intravenous, aerosol, parenteral, ophthalmic, pulmonary, and topical administration. In some embodiments, the imaging composition is formulated for intravenous administration.

As used herein, “subject” refers to any animal, including mammals such as primates (e.g., humans), mice, rats, other rodents, rabbits, dogs, cats, swine, cattle, sheep, horses, primates, and humans. In some embodiments, the subject is a human. In some embodiments, the subject has experienced and/or exhibited at least one symptom of the disease or disorder to be treated and/or prevented.

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

It is understood that atoms making up the compounds described herein are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C. In some embodiments, the compounds described herein are isotopically enriched, i.e., having greater than the naturally occurring proportion of a particular isotope. In some embodiments, the compounds described herein are radiolabeled, i.e., labeled with radioactive isotopes. Such compounds are referred to as “radiolabeled.” Examples of radioisotopes include, but are not limited to ¹¹C and ¹⁸F.

In addition, the compounds generically or specifically disclosed herein are intended to include all tautomeric forms. Thus, by way of example, a compound containing the moiety:

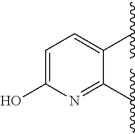 [SMILES: *c1ccc(O)nc1*]

encompasses the tautomeric form containing the moiety:

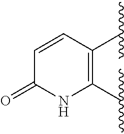 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms.

The compounds provided herein may encompass various stereochemical forms. The compounds also encompass enantiomers (e.g., R and S isomers), diastereomers, as well as mixtures of enantiomers (e.g., R and S isomers) including racemic mixtures and mixtures of diastereomers, as well as individual enantiomers and diastereomers, which arise as a consequence of structural asymmetry in certain compounds. Unless otherwise indicated, when a disclosed compound is named or depicted by a structure without specifying the stereochemistry (e.g., a “flat” structure) and has one or more chiral centers, it is understood to represent all possible stereoisomers of the compound. Likewise, unless otherwise indicated, when a disclosed compound is named or depicted by a structure that specifies the stereochemistry (e.g., a structure with “wedge” and/or “dashed” bonds) and has one or more chiral centers, it is understood to represent the indicated stereoisomer of the compound.

The details of one or more embodiments of this disclosure are set forth in the accompanying drawings and the description below. Other features and advantages of the present disclosure will be apparent from the description and drawings, and from the claims.

### Compounds

In some embodiments, the compound (i.e., an inhibitor of α-synuclein translation) is selected from the group consisting of:

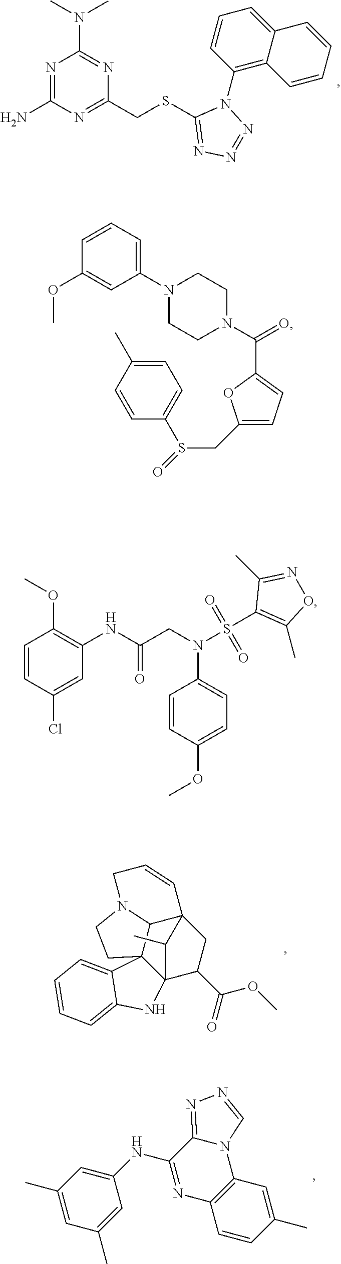

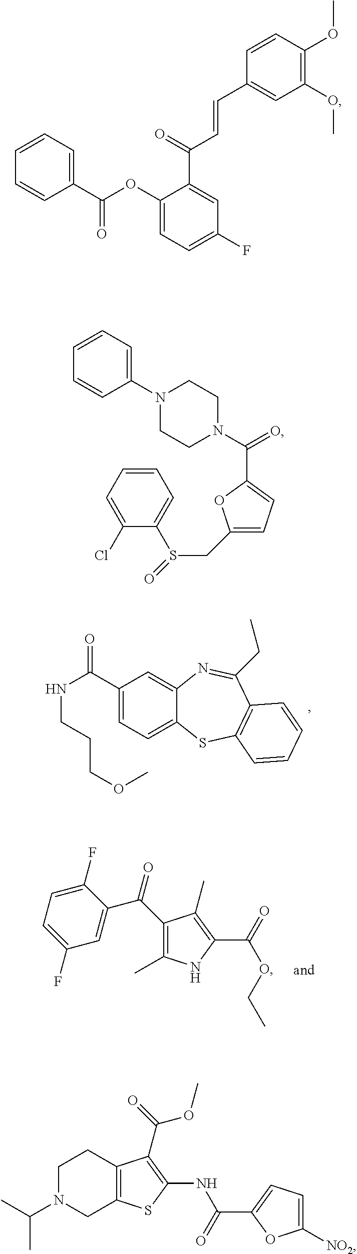

or a pharmaceutically acceptable salt of any of the foregoing.

In some embodiments, the compound (i.e., an inhibitor of α-synuclein translation) is selected from the group consisting of: N²,N²-dimethyl-6-(((1-(naphthalen-1-yl)-1H-tetrazol-5-yl)thio)methyl)-1,3,5-triazine-2,4-diamine, (4-(3-methoxyphenyl)piperazin-1-yl)(5-((p-tolylsulfinyl)methyl)furan-2-yl)methanone, N-(5-chloro-2-methoxyphenyl)-2-((N-(4-methoxyphenyl)-3,5-dimethylisoxazole)-4-sulfonamido)acetamide, methyl 14-methyl-4,5,11,12-tetrahydro-¹H,3a¹H,6H-3a,5a-methanoindolizino[8,1-cd]carbazole-5-carboxylate, N-(3,5-dimethylphenyl)-8-methyl-[1,2,4]triazolo[4,3-a]quinoxalin-4-amine, (E)-2-(3-(3,4-dimethoxyphenyl)acryloyl)-4-fluorophenyl benzoate, (5-(((2-chlorophenyl)sulfinyl)methyl)furan-2-yl)(4-phenylpiperazin-1-yl)methanone, 11-ethyl-N-(3-methoxypropyl)dibenzo[b,f][1,4]thiazepine-8-carboxamide, ethyl 4-(2,5-difluorobenzoyl)-3,5-dimethyl-1H-pyrrole-2-carboxylate, and methyl 6-isopropyl-2-(5-nitrofuran-2-carboxamido)-4,5,6,7-tetrahydrothieno[2,3-c]pyridine-3-carboxylate, or a pharmaceutically acceptable salt thereof.

In some embodiments, the compound is selected from the group consisting of:

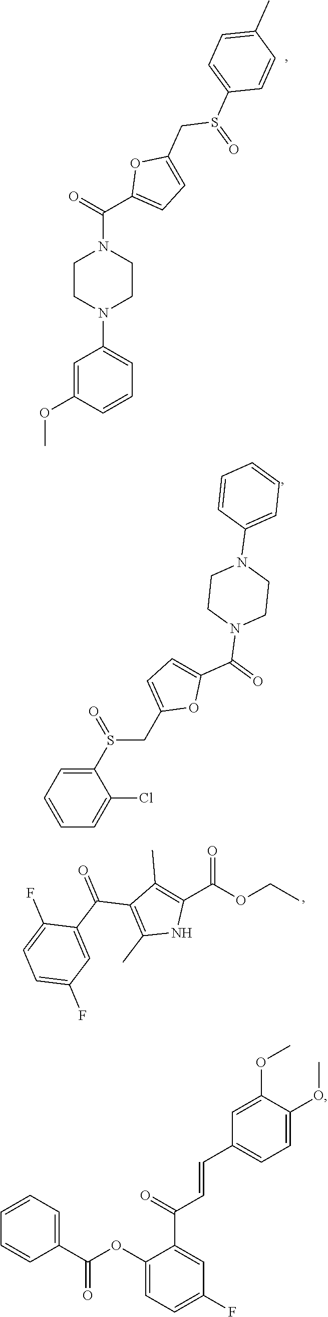

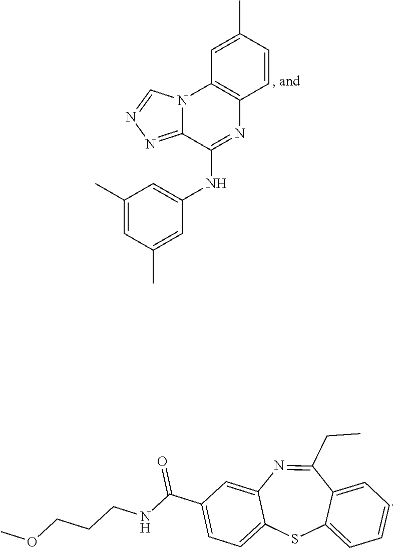 [SMILES: Cc1cc(C)cc(Nc2nc3ccc(C)cc3n3cnnc23)c1 ; CCC1=Nc2cc(C(=O)NCCCOC)ccc2Sc2ccccc21]

In some embodiments, the compound is selected from the group consisting of: 1-(3-Methoxyphenyl)-4-(5-{[(4-methylphenyl)sulfinyl]methyl}-2-furoyl)piperazine, 1-(5-{[(2-Chlorophenyl)sulfinyl]methyl}-2-furoyl)-4-phenylpiperazine, Ethyl 4-(2,5-difluorobenzoyl)-3,5-dimethyl-1H-pyrrole-2-carboxylat, 2-[(2E)-3-(3,4-dimethoxyphenyl)prop-2-enoyl]-4-fluorophenyl benzoate, N-(3,5-dimethylphenyl)-8-methyl[1,2,4]triazolo[4,3-a]quinoxalin-4-amine, and 11-ethyl-N-8-(3-methoxypropyl)dibenzo[b,f][1,4]thiazepine-8-carboxamide.

Some embodiments provide a radiolabeled compound, or a pharmaceutically acceptable salt thereof, selected from the group consisting of:

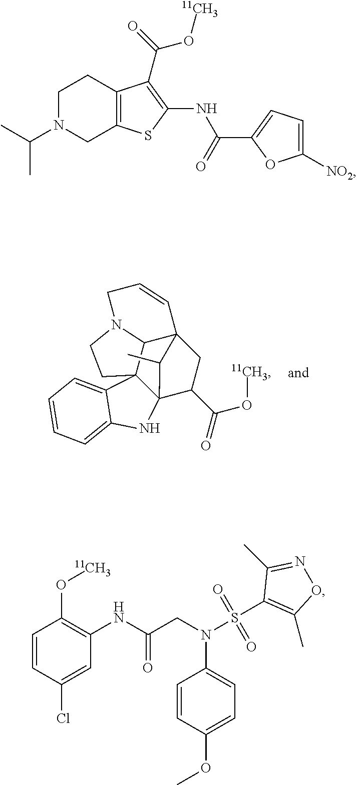

or a pharmaceutically acceptable salt of any of the foregoing. In some embodiments of the compositions and methods described herein, the compound is a radiolabeled compound, or a pharmaceutically acceptable salt thereof, selected from the group consisting of:

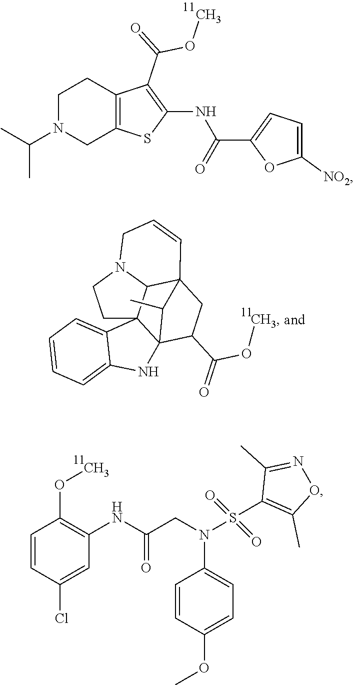

or a pharmaceutically acceptable salt of any of the foregoing.

In some embodiments, the compound (i.e., an inhibitor of α-synuclein translation) is selected from the group consisting of: methyl-¹¹C 6-isopropyl-2-(5-nitrofuran-2-carboxamido)-4,5,6,7-tetrahydrothieno[2,3-c]pyridine-3-carboxylate, methyl-¹¹C 14-methyl-4,5,11,12-tetrahydro-1H,3a¹H,6H-3a,5a-methanoindolizino[8,1-cd]carbazole-5-carboxylate, and N-(5-chloro-2-(methoxy-¹¹C)phenyl)-2-((N-(4-methoxyphenyl)-3,5-dimethylisoxazole)-4-sulfonamido)acetamide, or a pharmaceutically acceptable salt thereof.

In some embodiments, the compound is

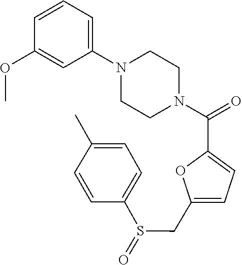 [SMILES: COc1cccc(N2CCN(C(=O)c3ccc(CS(=O)c4ccc(C)cc4)o3)CC2)c1]

[(4-(3-methoxyphenyl)piperazin-1-yl)(5-((p-tolylsulfinyl)methyl)furan-2-yl)methanone], or a pharmaceutically acceptable salt thereof.

In some embodiments, the compound is

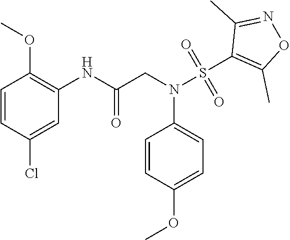 [SMILES: COc1ccc(N(CC(=O)Nc2cc(Cl)ccc2OC)S(=O)(=O)c2c(C)noc2C)cc1]

[N-(5-chloro-2-methoxyphenyl)-2-((N-(4-methoxyphenyl)-3,5-dimethylisoxazole)-4-sulfonamido)acetamide], or a pharmaceutically acceptable salt thereof.

In some embodiments, the compound is

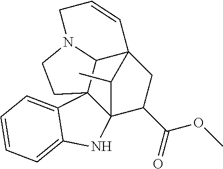

[methyl 14-methyl-4,5,11,12-tetrahydro-1H,3a¹H,6H-3a,5a-methanoindolizino[8,1-cd]carbazole-5-carboxylate], or a pharmaceutically acceptable salt thereof.

In some embodiments, the compound is

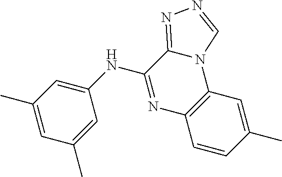

[N-(3,5-dimethylphenyl)-8-methyl-[1,2,4]triazolo[4,3-a]quinoxalin-4-amine], or a pharmaceutically acceptable salt thereof.

In some embodiments, the compound is

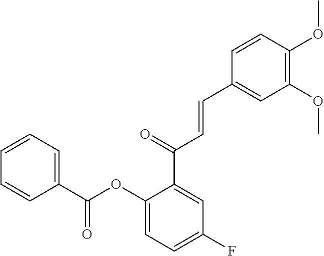 [SMILES: COc1ccc(/C=C/C(=O)c2cc(F)ccc2OC(=O)c2ccccc2)cc1OC]

[(E)-2-(3-(3,4-dimethoxyphenyl)acryloyl)-4-fluorophenyl benzoate], or a pharmaceutically acceptable salt thereof.

In some embodiments, the compound is

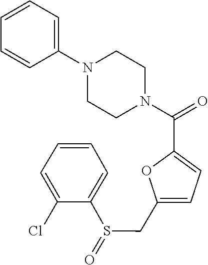 [SMILES: O=C(c1ccc(CS(=O)c2ccccc2Cl)o1)N1CCN(c2ccccc2)CC1]

[(5-(((2-chlorophenyl)sulfinyl)methyl)furan-2-yl)(4-phenylpiperazin-1-yl)methanone], or a pharmaceutically acceptable salt thereof.

In some embodiments, the compound is

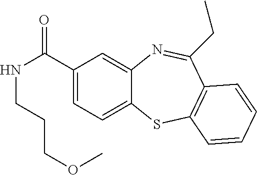 [SMILES: CCC1=Nc2cc(C(=O)NCCCOC)ccc2Sc2ccccc21]

[11-ethyl-N-(3-methoxypropyl)dibenzo[b,f][1,4]thiazepine-8-carboxamide], or a pharmaceutically acceptable salt thereof.

In some embodiments, the compound is

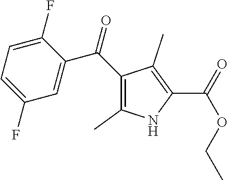 [SMILES: CCOC(=O)c1[nH]c(C)c(C(=O)c2cc(F)ccc2F)c1C]

[ethyl 4-(2,5-difluorobenzoyl)-3,5-dimethyl-1H-pyrrole-2-carboxylate], or a pharmaceutically acceptable salt thereof.

In some embodiments, the compound is

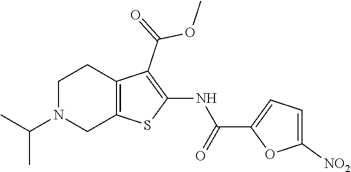 [SMILES: COC(=O)c1c(NC(=O)c2ccc([N+](=O)[O-])o2)sc2c1CCN(C(C)C)C2]

[methyl 6-isopropyl-2-(5-nitrofuran-2-carboxamido)-4,5,6,7-tetrahydrothieno[2,3-c]pyridine-3-carboxylate], or a pharmaceutically acceptable salt thereof.

In some embodiments, the compound is

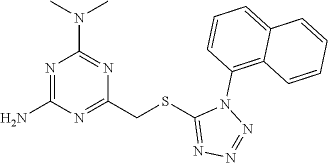 [SMILES: CN(C)c1nc(N)nc(CSc2nnnn2-c2cccc3ccccc23)n1]

[N²,N²-dimethyl-6-(((1-(naphthalen-1-yl)-1H-tetrazol-5-yl)thio)methyl)-1,3,5-triazine-2,4-diamine], or a pharmaceutically acceptable salt thereof.

In some embodiments, the compound is

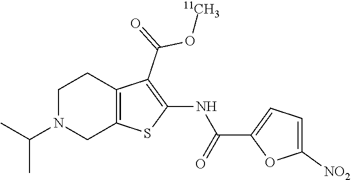 [SMILES: CC(C)N1CCc2c(sc(NC(=O)c3ccc([N+](=O)[O-])o3)c2C(=O)O[11CH3])C1]

[methyl-¹¹C 6-isopropyl-2-(5-nitrofuran-2-carboxamido)-4,5,6,7-tetrahydrothieno[2,3-c]pyridine-3-carboxylate], or a pharmaceutically acceptable salt thereof.

In some embodiments, the compound is

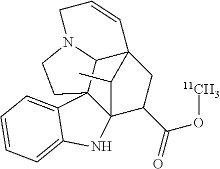

[methyl-¹¹C 14-methyl-4,5,11,12-tetrahydro-1H,3a¹H,6H-3a,5a-methanoindolizino[8,1-cd]carbazole-5-carboxylate], or a pharmaceutically acceptable salt thereof.

In some embodiments, the compound is

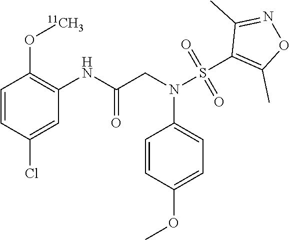 [SMILES: COc1ccc(N(CC(=O)Nc2cc(Cl)ccc2O[11CH3])S(=O)(=O)c2c(C)noc2C)cc1]

[N-(5-chloro-2-(methoxy-¹¹C)phenyl)-2-((N-(4-methoxyphenyl)-3,5-dimethylisoxazole)-4-sulfonamido)acetamide], or a pharmaceutically acceptable salt thereof.

In some embodiments, the compound is

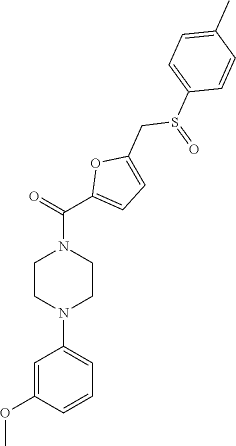 [SMILES: COc1cccc(N2CCN(C(=O)c3ccc(CS(=O)c4ccc(C)cc4)o3)CC2)c1]

1-(3-Methoxyphenyl)-4-(5-{[(4-methylphenyl)sulfinyl]methyl}-2-furoyl)piperazine, or a pharmaceutically acceptable salt thereof.

In some embodiments, the compound is

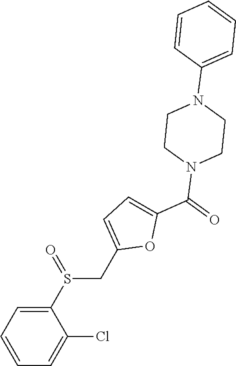 [SMILES: O=C(c1ccc(CS(=O)c2ccccc2Cl)o1)N1CCN(c2ccccc2)CC1]

1-(5-{[(2-Chlorophenyl)sulfinyl]methyl}-2-furoyl)-4-phenylpiperazine, or a pharmaceutically acceptable salt thereof.

In some embodiments, the compound is

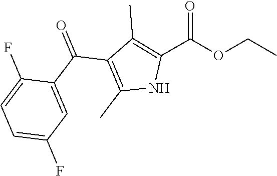 [SMILES: CCOC(=O)c1[nH]c(C)c(C(=O)c2cc(F)ccc2F)c1C]

Ethyl 4-(2,5-difluorobenzoyl)-3,5-dimethyl-1H-pyrrole-2-carboxylat, or a pharmaceutically acceptable salt thereof.

In some embodiments, the compound is

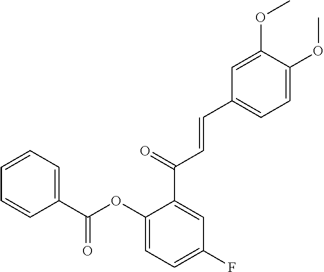 [SMILES: COc1ccc(/C=C/C(=O)c2cc(F)ccc2OC(=O)c2ccccc2)cc1OC]

2-[(2E)-3-(3,4-dimethoxyphenyl)prop-2-enoyl]-4-fluorophenyl benzoate, or a pharmaceutically acceptable salt thereof.

In some embodiments, the compound is

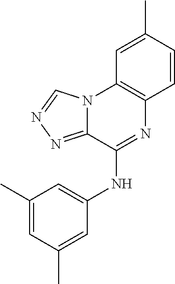 [SMILES: Cc1cc(C)cc(Nc2nc3ccc(C)cc3n3cnnc23)c1]

N-(3,5-dimethylphenyl)-8-methyl[1,2,4]triazolo[4,3-a]quinoxalin-4-amine, or a pharmaceutically acceptable salt thereof.

In some embodiments, the compound is

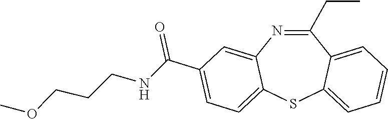 [SMILES: CCC1=Nc2cc(C(=O)NCCCOC)ccc2Sc2ccccc21]

11-ethyl-N˜8˜-(3-methoxypropyl)dibenzo[b,f][1,4]thiazepine-8-carboxamide, or a pharmaceutically acceptable salt thereof.

### Pharmaceutical Compositions and Administration

### General

In some embodiments, a chemical entity (e.g., any of Compounds 1-16, or a pharmaceutically acceptable salt thereof) is administered as a pharmaceutical composition that includes the chemical entity and one or more pharmaceutically acceptable excipients, and optionally one or more additional therapeutic agents as described herein.

In some embodiments, the chemical entities can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from pharmaceutically acceptable excipients may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, UK. 2012).

### Routes of Administration and Composition Components

In some embodiments, the chemical entities described herein or a pharmaceutical composition thereof can be administered to subject in need thereof by any accepted route of administration. Acceptable routes of administration include, but are not limited to, buccal, cutaneous, enteral, epidural, interstitial, intra-abdominal, intra-arterial, intrabronchial, intracerebral, intracisternal, intradural, intraepidermal, intraesophageal, intramedullary, intrameningeal, intramuscular, intrasinal, intraspinal, intrathecal, intravascular, intravenous, nasal, oral, parenteral, percutaneous, peridural, respiratory (inhalation), subcutaneous, sublingual, submucosal, topical, transdermal, and transmucosal. In certain embodiments, a preferred route of administration is parenteral (e.g., intravenous).

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

In other embodiments, the compounds described herein or a pharmaceutical composition thereof are suitable for local delivery to the digestive or GI tract by way of oral administration (e.g., solid or liquid dosage forms.).

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such pharmaceutically acceptable excipients as lactose or milk sugar as well as high molecular weight polyethylene glycols and the like.

In one embodiment, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments, the pharmaceutically acceptable excipients are sterile and generally free of undesirable matter. These compositions can be sterilized by conventional, well-known sterilization techniques. For various oral dosage forms, pharmaceutically acceptable excipients such as tablets and capsules sterility is not required. The USP/NF standard is usually sufficient.

In some embodiments, the compounds described herein, or pharmaceutically acceptable salts thereof, are administered orally in the form of capsules or tablets, for example, or as an aqueous suspension or solution. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening and/or flavoring and/or coloring agents may be added.

In certain embodiments, solid oral dosage forms can further include one or more components that chemically and/or structurally predispose the composition for delivery of the chemical entity to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802, which is incorporated herein by reference in its entirety.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and pharmaceutically acceptable excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

In some embodiments, the pharmaceutical compositions are in the form of a sterile injectable preparation, for example as a sterile injectable aqueous or oleaginous suspension. This suspension may be formulated according to techniques know in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. Fatty acids such as oleic acid and its glyceride derivatives find use in the preparation of injectables, olive oil or castor oil, especially in their polyoxyethylated versions. These oil solutions or suspensions may also contain a long-chain alcohol diluent or dispersant.

In any of the foregoing embodiments, pharmaceutical compositions described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

### Dosages

In some embodiments, dosage levels on the order of about 0.01 mg to about 10,000 mg of the active ingredient compound are useful in the treatment of the disease or disorder described herein. In some embodiments, dosage levels on the order of about 0.1 mg to about 1,000 mg of the active ingredient compound are useful in the treatment of the disease or disorder described herein. In some embodiments, the dosage level is from about 1 mg to about 500 mg. The amount of active ingredient that may be combined with the carrier materials to produce a single dosage form will vary depending upon the host treated and the particular mode of administration. However, that a specific dose level for any particular patient will depend upon a variety of factors including the activity of the specific compound employed, the age, body weight, general health, sex, diet, time of administration, rate of excretion, drug combination, and the severity of the particular disease being treated and form of administration. The total daily dosage may be divided and administered in portions throughout the day or by means providing continuous delivery.

### Methods of Treatment

### Indications

Some embodiments provide a method for treating Parkinson's disease in a subject in need thereof, comprising administering to the subject a therapeutically effective amount of any one of Compounds 1-16, or a pharmaceutically acceptable salt thereof, or a pharmaceutical composition as provided herein.

Some embodiments provide a method for treating dementia with Lewy bodies in a subject in need thereof, comprising administering to the subject a therapeutically effective amount of any one of Compounds 1-16, or a pharmaceutically acceptable salt thereof, or a pharmaceutical composition as provided herein.

Some embodiments provide a method for treating multiple systems atrophy in a subject in need thereof, comprising administering to the subject a therapeutically effective amount of any one of Compounds 1-16, or a pharmaceutically acceptable salt thereof, or a pharmaceutical composition as provided herein.

Some embodiments provide a method for treating prodromal REM sleep behavior disorder (RBD) in a subject in need thereof, comprising administering to the subject a therapeutically effective amount of any one of Compounds 1-16, or a pharmaceutically acceptable salt thereof, or a pharmaceutical composition as provided herein.

Some embodiments provide a method of treating an α-synuclein associated disease or disorder in a subject, comprising administering to a subject identified or diagnosed as having an α-synuclein associated disease or disorder a therapeutically effective amount of any one of Compounds 1-16, or a pharmaceutically acceptable salt thereof, or a pharmaceutical composition as provided herein.

Some embodiments provide a method of treating an α-synuclein associated disease or disorder in a subject, comprising: determining that the disease or disorder in the subject is an α-synuclein associated disease or disorder; and administering to the subject a therapeutically effective amount of any one of Compounds 1-16, or a pharmaceutically acceptable salt thereof, or a pharmaceutical composition as provided herein.

In some embodiments, the determining that the disease or disorder in the subject is an α-synuclein associated disease or disorder comprises a genetic test. In some embodiments, the determining that the disease or disorder in the subject is an α-synuclein associated disease or disorder comprises diagnostic imaging. In some embodiments, the determining that the disease or disorder in the subject is an α-synuclein associated disease or disorder comprises neurological examination.

Some embodiments provide a method of treating a subject, comprising administering a therapeutically effective amount of any one of Compounds 1-16, or a pharmaceutically acceptable salt thereof, or a pharmaceutical composition as provided herein, to a subject having a clinical record that indicates that the subject has an α-synuclein associated disease or disorder.

In some embodiments, the subject is a human.

The ability of test compounds to act as inhibitors of α-synuclein translation may be demonstrated by assays known in the art. The activity of the compounds and compositions provided herein as α-synuclein translation inhibitors can be assayed in vitro, in vivo, or in a cell line.

Potency of an α-synuclein translation inhibitor as provided herein can be determined by EC₅₀ value. A compound with a lower EC₅₀ value, as determined under substantially similar conditions, is a more potent inhibitor relative to a compound with a higher EC₅₀ value.

Potency of an α-synuclein translation inhibitor as provided herein can also be determined by IC₅₀ value. A compound with a lower IC₅₀ value, as determined under substantially similar conditions, is a more potent inhibitor relative to a compound with a higher IC₅₀ value.

In some embodiments, the compound is one of Compounds 1-10, or a pharmaceutically acceptable salt thereof. In some embodiments, the compound is one of Compounds 11-16, or a pharmaceutically acceptable salt thereof.

### Combinations

In some embodiments, the compounds described herein are administered with one or more additional anti-Parkinson's agents. In some embodiments, the compounds described herein are administered with one or two additional anti-Parkinson's agents. In some embodiments, the subject was previously administered one or more additional anti-Parkinson's agents, and is no longer being administered the one or more anti-Parkinson's agents.

Anti-Parkinson's agents include, but are not limited to chelators, posiphen, levodopa, entacopone, tolcapone, pramipexole, ropinirole, apomorphine, benztropine, rotigotine, selegiline, rasagiline, safinamide, amantadine, istradefylline and/or trihexyphenidyl.

Chelators include, but are not limited to iron chelators such as desferroxamine and deferiprone. In some embodiments, the chelator is desferroxamine or deferiprone.

The dosage level of other drugs will depend upon the factors previously stated and the effectiveness of the drug combination.

### Methods of Imaging

Some embodiments provide a method for detecting an α-synuclein associated disease or disorder in a subject in need thereof, comprising administering to the subject an effective amount of any one of Compounds 1-16, or a pharmaceutically acceptable salt of any of the foregoing; wherein the compound, or a pharmaceutically acceptable salt thereof, is radiolabeled.

Some embodiments provide a method for detecting Parkinson's disease in a subject in need thereof, comprising administering to the subject an effective amount of an radiolabeled compound described herein, or a pharmaceutically acceptable salt thereof.

Some embodiments provide a method for detecting dementia with Lewy bodies in a subject in need thereof, comprising administering to the subject an effective amount of an radiolabeled compound described herein, or a pharmaceutically acceptable salt thereof.

Some embodiments provide a method for detecting multiple systems atrophy in a subject in need thereof, comprising administering to the subject an effective amount of an radiolabeled compound described herein, or a pharmaceutically acceptable salt thereof.

Some embodiments provide a method for detecting prodromal REM sleep behavior disorder (RBD) in a subject in need thereof, comprising administering to the subject an effective amount of an radiolabeled compound described herein, or a pharmaceutically acceptable salt thereof.

Some embodiments provide a method of detecting an α-synuclein associated disease or disorder in a subject, comprising administering to a subject suspected as having an α-synuclein associated disease or disorder an effective amount of an radiolabeled compound described herein, or a pharmaceutically acceptable salt thereof.

Some embodiments provide a method for detecting α-synuclein translation in a mammalian cell, comprising contacting the mammalian cell with an effective amount of an radiolabeled compound described herein, or a pharmaceutically acceptable salt thereof.

Some embodiments provide a method for detecting Lewy body formation in mammalian brain tissue, comprising contacting the mammalian brain tissue with an effective amount of an radiolabeled compound described herein, or a pharmaceutically acceptable salt thereof.

In some embodiments, the detecting comprises (a) administering an effective amount of an imaging composition, as described herein, to a subject in need thereof; and (b) after a period of time, conducting a PET or SPECT scan.

In some embodiments, the period of time is from about 1 minute to about 60 minutes, for example, about 1 minute to about 15 minutes, about 5 minutes to about 20 minutes, about 10 minutes to about 30 minutes, about 15 minutes to about 40 minutes, about 20 minutes to about 45 minutes, about 30 minutes to about 50 minutes, about 40 minutes to about 60 minutes.

In some embodiments, the compound is one of Compounds 1-10, or a pharmaceutically acceptable salt thereof. In some embodiments, the compound is one of Compounds 11-16, or a pharmaceutically acceptable salt thereof.

### EXAMPLES

### Compound Preparation

The compounds disclosed herein can be prepared in a variety of ways using commercially available starting materials, compounds known in the literature, or from readily prepared intermediates, by employing standard synthetic methods and procedures either known to those skilled in the art, or in light of the teachings herein. The synthesis of the compounds disclosed herein can be achieved by generally following the schemes provided herein, with modification for specific desired substituents.

Standard synthetic methods and procedures for the preparation of organic molecules and functional group transformations and manipulations can be obtained from the relevant scientific literature or from standard textbooks in the field. Although not limited to any one or several sources, classic texts such as R. Larock, Comprehensive Organic Transformations, VCH Publishers (1989); L. Fieser and M. Fieser, Fieser and Fieser's Reagents for Organic Synthesis, John Wiley and Sons (1994); Smith, M. B., March, J., March's Advanced Organic Chemistry: Reactions, Mechanisms, and Structure, 5th edition, John Wiley & Sons: New York, 2001; and Greene, T. W., Wuts, P. G. M., Protective Groups in Organic Synthesis, 3rd edition, John Wiley & Sons: New York, 1999, are useful and recognized reference textbooks of organic synthesis known to those in the art. The following descriptions of synthetic methods are designed to illustrate, but not to limit, general procedures for the preparation of compounds of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt thereof.

Compounds 1-16 as described herein are readily available from commercial suppliers.

### Example 1. Synthesis of methyl-¹¹C 6-isopropyl-2-(5-nitrofuran-2-carboxamido)-4,5,6,7-tetrahydrothieno[2,3-c]pyridine-3-carboxylate (compound 9-r)

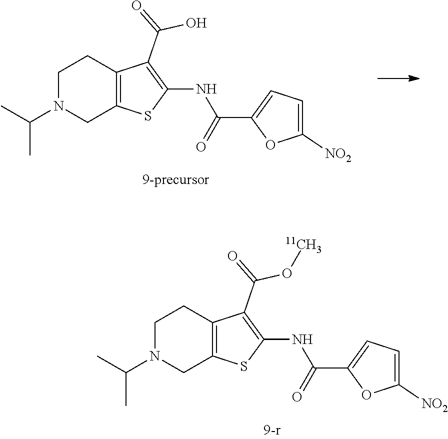 [SMILES: CC(C)N1CCc2c(sc(NC(=O)c3ccc([N+](=O)[O-])o3)c2C(=O)O)C1 ; CC(C)N1CCc2c(sc(NC(=O)c3ccc([N+](=O)[O-])o3)c2C(=O)O[11CH3])C1]

[¹¹C]CO₂ was obtained via the ¹⁴N (p, α) ¹¹C reaction on nitrogen with 2.5% oxygen, with 11 MeV protons (Siemens Eclipse cyclotron), and trapped on molecular sieves in a TRACERlab FX-MeI synthesizer (General Electric). [¹¹C]CH₄ was obtained by the reduction of [¹¹C]CO₂ in the presence of Ni/hydrogen at 350° C., which re-circulated through an oven containing 12 to produce [¹¹C]CH₃I via a radical reaction. The prepared [¹¹C]CH₃I was trapped in anhydrous DMF (300 μL) containing 9-precursor (1.0 mg) and K₂CO₃ (5.0 mg). The reaction vessel was heated at 80° C. and kept there for 3 min. The radioactive mixture containing [¹¹C]CH₃I was quenched by addition of an HPLC mobile phase (0.7 mL) and then applied to a reverse phase semipreparative HPLC (Phenomenex Gemini-NX 5u C18 110 A, 250×10 mm, 5.0 mL/min, 60% CH₃CN+0.1% TFA/40% H₂O+0.1% TFA). A radioactive fraction having a retention time of 9 min was collected in a flask, and diluted in water (30 mL).

The final product was reformulated by loading onto a solid-phase exchange (SPE) C-18 cartridge (Waters WAT020515 Sep-Pak Plus Short C18), rinsing with water (4×5 mL), eluting with EtOH (0.3 mL), and diluting with saline (2.7 mL). The chemical and radiochemical purity of the final product was tested by analytical HPLC (VARIAN Puruit XRs 5 C18, 150×4.6 mm), eluting with a gradient of 10-90% CH₃CN in H₂O of 0.1% TFA, at a flow rate of 2 mL/min. Confirmation of the identity of 9-r was achieved by co-injection with standard compound as reference standard. Radiochemical yield: 20-35% (decay non-corrected to C11-CH₃I) with both radioactive and UV purity>95%.

### Example 2. Synthesis of methyl-¹¹C 14-methyl-4,5,11,12-tetrahydro-1H,3a¹H,6H-3a,5a-methanoindolizino[8,1-cd]carbazole-5-carboxylate (compound 3-r)

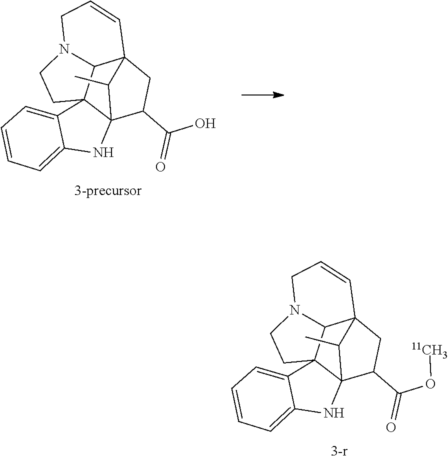

[¹¹C]CO₂ was obtained via the ¹⁴N (p, α) ¹¹C reaction on nitrogen with 2.5% oxygen, with 11 MeV protons (Siemens Eclipse cyclotron), and trapped on molecular sieves in a TRACERlab FX-MeI synthesizer (General Electric). [¹¹C]CH₄ was obtained by the reduction of [¹¹C]CO₂ in the presence of Ni/hydrogen at 350° C., which re-circulated through an oven containing 12 to produce [¹¹C]CH₃I via a radical reaction. The prepared [¹¹C]CH₃I was trapped in anhydrous DMF (300 μL) containing 3-precursor (1.0 mg) and K₂CO₃ (5.0 mg). The reaction vessel was heated at 80° C. and kept there for 3 min. The radioactive mixture containing [¹¹C]CH₃I was quenched by addition of an HPLC mobile phase (0.7 mL) and then applied to a reverse phase semipreparative HPLC (Phenomenex Gemini-NX 5u C18 110 A, 250×10 mm, 5.0 mL/min, 70% CH₃CN+0.1% TFA/30% H₂O+0.1% TFA). A radioactive f

The final product was reformulated by loading onto a solid-phase exchange (SPE) C-18 cartridge (Waters WAT020515 Sep-Pak Plus Short C18), rinsing with water (4×5 mL), eluting with EtOH (0.3 mL), and diluting with saline (2.7 mL). The chemical and radiochemical purity of the final product was tested by analytical HPLC (VARIAN Puruit XRs 5 C18, 150×4.6 mm), eluting with a gradient of 10-90% CH₃CN in H₂O of 0.1% TFA, at a flow rate of 2 mL/min. Confirmation of the identity of 3-r was achieved by co-injection with standard compound as reference standard. Radiochemical yield: 30-35% (decay non-corrected to C11-CH₃I) with both radioactive and UV purity>95%.

### Example 3. Synthesis of N-(5-chloro-2-(methoxy-¹¹C)phenyl)-2-((N-(4-methoxyphenyl)-3,5-dimethylisoxazole)-4-sulfonamido)acetamide (compound 2-r)

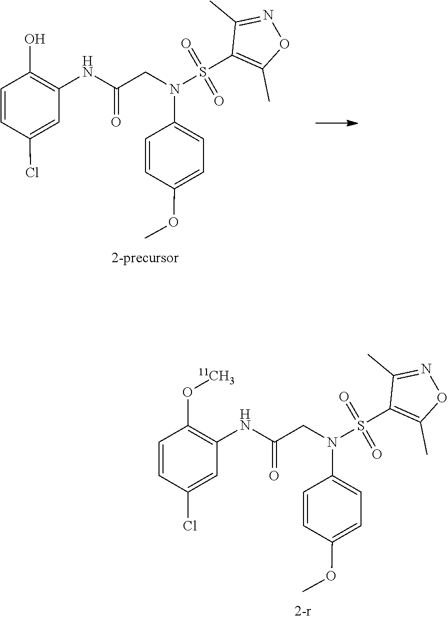 [SMILES: COc1ccc(N(CC(=O)Nc2cc(Cl)ccc2O)S(=O)(=O)c2c(C)noc2C)cc1 ; COc1ccc(N(CC(=O)Nc2cc(Cl)ccc2O[11CH3])S(=O)(=O)c2c(C)noc2C)cc1]

[¹¹C]CO₂ was obtained via the ¹⁴N (p, α) ¹¹C reaction on nitrogen with 2.5% oxygen, with 11 MeV protons (Siemens Eclipse cyclotron), and trapped on molecular sieves in a TRACERlab FX-MeI synthesizer (General Electric). [¹¹C]CH₄ was obtained by the reduction of [¹¹C]CO₂ in the presence of Ni/hydrogen at 350° C., which re-circulated through an oven containing 12 to produce [¹¹C]CH₃I via a radical reaction. The prepared [¹¹C]CH₃I was trapped in anhydrous DMF (300 μL) containing 2-precursor (1.0 mg) and K₂CO₃ (5.0 mg). The reaction vessel was heated at 80° C. and kept there for 3 min. The radioactive mixture containing [¹¹C]CH₃I was quenched by addition of an HPLC mobile phase (0.7 mL) and then applied to a reverse phase semipreparative HPLC (Phenomenex Gemini-NX 5u C18 110 A, 250×10 mm, 5.0 mL/min, 70% CH₃CN+0.1% TFA/30% H₂O+0.1% TFA). A radioactive fraction having a retention time of 11 min was collected in a flask, and diluted in water (30 mL).

The final product was reformulated by loading onto a solid-phase exchange (SPE) C-18 cartridge (Waters WAT020515 Sep-Pak Plus Short C18), rinsing with water (4×5 mL), eluting with EtOH (0.3 mL), and diluting with saline (2.7 mL). The chemical and radiochemical purity of the final product was tested by analytical HPLC (VARIAN Puruit XRs 5 C18, 150×4.6 mm), eluting with a gradient of 10-90% CH₃CN in H₂O of 0.1% TFA, at a flow rate of 2 mL/min. Confirmation of the identity of 2-r was achieved by co-injection with standard compound as reference standard. Radiochemical yield: 30-35% (decay non-corrected to C11-CH₃I) with both radioactive and UV purity>95%.

### Biological Assays

### Materials and Reagents

Dulbeccos modified essential medium (DMEM, catalog no. 12-614Q); FBS (catalog no. 14-503E), L-glutamine (catalog no. 17-605E), penicillin/streptomycin (catalog no. 17-602E); phenol red free media (phenol red-free DMEM with 4.5 g/L D-glucose (catalog no. 12-917F) were purchased from Lonza (Portsmouth, NH). Geneticin (catalog no. 10131-027)) and alpha-synuclein ELISA kit KHB0061 were acquired from Invitrogen. Trypsin/EDTA (catalog no. 25-053-C1) was purchased from Cellgro, and strophanthidine (catalog no. S6626-250MG, Lot 038K1036) was purchased from Sigma. Steady-Glo (catalog no. E2250) was purchased from Promega (Madison, WI). For the Western Blot secondary screen, penicillin/streptomycin was acquired from Bio-Whittaker (Walkerville, MD), mouse monoclonal anti-alpha-synuclein was purchased from BD Transduction Laboratories, and anti-beta-actin was acquired from Chemicon. 384-well plates (catalog no. 3570) were purchased from Corning and Falcon TC flasks (catalog no. 353112) were purchased from Becton Dickison (Waltham, MA).

### Protocols

### Screening Protocols

### Screening Protocol 1: Primary Screen for ASYN 5′-UTR IRE-Driven Luciferase Expression in H4 Cells

Stably transfected H4 neuroglioblastoma cells were grown to confluency in 35 mL complete DMEM with 4.5 g/L D-glucose supplemented with 10% FBS, 200 μM L-glutamine, 100 μM penicillin/streptomycin, and 200 μg/mL geneticin in a T175 TC flask in a TC incubator (37° C., 95% humidity, 5% CO₂) (doubling time=24 h). Cells were harvested by washing the monolayer quickly with 5 mL trypsin/EDTA (1×), aspirating, then adding 5 mL trypsin/EDTA and incubating for 5 min at 37° C., 95% humidity, 5% CO₂. Then, 5 mL of complete media was added and mixed by pipetting up and down. Then, 10 mL of cell solution was aspirated and added to a conical tube; cells were pelleted for 3 min at 1000 rpm in a swinging bucket centrifuge. The media was aspirated, and the cell pellet was resuspended in 10 mL fresh complete media. Cells were then counted and expanded by plating 1×10⁶ cells in 35 mL complete media per T175 flask (20×T175 flasks per 200 assay plate run) and allowing 72 h for cells to grow to confluency. Cells were harvested as above, but were resuspended in phenol-red free complete media (phenol red-free DMEM with 4.5 g/L D-glucose supplemented with 10% FBS, 200 μM L-glutamine, 100 μM penstrep, and 200 μg/mL geneticin, and then were diluted to 60,000 cells/mL in phenol-red free complete media prewarmed to 37° C. in 1-liter sterile plastic bottles. Cells were plated into white TC treated 384-well plates (batch size=200-220 assay plates) at 3,000 cells/well in 50 μL phenol-red free complete media using a Thermo MultiDrop Combi liquid dispenser and a sterilized dispensing cassette and stir bar, in a TC hood. Assay plates were loaded into 22 slot holders which were then placed into an online Liconic (STX 2201C) incubator set to 37° C., 95% humidity, 5% CO₂, and were incubated overnight.

Screening was performed using an open system (HiRes Biosolutions). Each run was initiated in CBIP (Broad Chemical Biology Informatics Platform) and scheduled with Cellario software (HiRes Biosolutions). Staubli arms moved plates from different instruments on the robotic system. Replicate assay plates were delidded and then pinned with 100 nL of 3.75 mM compound (final concentration=7.5 μM) each with a 100 nL pin head using a MicroPin pin tool (HiRes Biosolutions). The positive control used in this assay was strophanthidine (Sigma, Cat. S6626-250MG, Lot038K1036), which was used in dose (12-point, 2-fold dilution, from 20 μM final [compound]), and was incorporated at the beginning and end of each run. Plates with compound were then relidded and returned to the Liconic incubator (STX 2201C) and incubated for 48 h at 37° C., 95% humidity, 5% CO₂. Plates were then individually moved to a room temperature Liconic Carousel (LPX 220) and were allowed to temperature equilibrate for 30 min. Plates were then delidded and moved to a MultiDrop Combi liquid dispenser where 30 μL of 0.5× Steady-Glo was added (Steady-Glo was maintained at 4° C. and warmed to room temperature using a Combi cassette with a 6-foot input line submerged in a room temperature water bath). Plates were then relidded, returned to the Liconic Carousel, and incubated at room temperature for 30 min. Plates were then delidded and moved to an Envision plate reader (Perkin Elmer); luminescence values were collected using with 100 ms read time per well. Plates were then relidded and discarded.

The specific protocol is as follows:

1) Grow stably transfected H4 neuroglioblastoma cells to confluency in 35 mL complete media (DMEM with 4.5 g/L D-glucose [Lonza, 12-6140] supplemented with 10% FBS [Lonza, 14-503E],

200 μM L-glutamine [Lonza, 17-605E], 100 μM penstrep [Lonza, 17-602E] and 200 μg/mL geneticin [Invitrogen, 10131-027]) in a T175 TC flask (BD Falcon, 353112).

2) Incubate in a TC incubator (37° C., 95% humidity, 5% CO₂) (doubling time=24 h).

3) Harvest cells by washing the monolayer quickly with 5 mL trypsin/EDTA (1×, Cellgro, 25-053-C1). Aspirate, then add 5 mL trypsin/EDTA.

4) Incubate for 5 min (37° C., 95% humidity, 5% CO₂).

5) Add 5 mL of complete media and mix by pipetting up and down. Aspirate 10 mL of cell solution and add to a conical tube.

6) Pellet the cells for 3 min at 1000 rpm in a swinging bucket centrifuge.

7) Aspirate the media and resuspend the cell pellet in 10 mL fresh complete media.

8) Count the cells and expand by plating 1×106 cells in 35 mL complete media per T175 flask (20×T175 flasks per 200 assay plate run). Allowing 72 h for cells to grow to confluency.

9) Harvest the cells as above, but resuspend cells in phenol-red free complete media (phenol red-free DMEM with 4.5 g/L D-glucose [Lonza, 12-917F] supplemented with 10% FBS [Lonza, 14-503E], 200 μM L-glutamine [Lonza, 17-605E], 100 μM penstrep [Lonza, 17-602E], and 200 μg/mL geneticin [Invitrogen, 10131-027]).

10) Dilute resuspended cells to 60,000 cells/mL in phenol-red free complete media prewarmed to 37° C. in 1-L sterile plastic bottles.

11) Plate cells into white TC-treated 384-well plates (Corning, 3570; batch size=200-220 assay plates) at 3,000 cells/well in 50-μL phenol-red free complete media using a Thermo MultiDrop Combi liquid dispenser, a sterilized dispensing cassette, stir bar in a TC hood.

12) Load assay plates into 22 slot holder and place into an online Liconic (STX 2201C) incubator set to 37° C., 95% humidity, 5% CO₂. Incubate plates overnight.

13) Perform screening using an open system (HiRes Biosolutions). Initiate each run in Broad Chemical Biology Informatics Platform (CBIP) and schedule with Cellario software (HiRes Biosolutions). Staubli arms moved plates from different instruments on the robotic system.

14) Delid replicate assay plates and pin with 100 nL of 3.75 μM compound (final concentration=7.5 μM) each with a 100 nL pin head using a MicroPin pin tool (HiRes Biosolutions).

15) Use strophanthidine (Sigma, Cat. #S6626-250MG, Lot #038K₁₀₃₆) as the positive control in dose (12-point, 2-fold dilution, from 20 μM final [compound]) at the beginning and end of each run. Relid plates with compound and return to the Liconic incubator (STX 2201C).

16) Incubate plates for 48 h at 37° C., 95% humidity, 5% CO₂. Move plates individually to a room temperature Liconic Carousel (LPX 220) and allow to temperature equilibrate for 30 min.

17) Delid plates and move to a MultiDrop Combi liquid dispenser and add 30 μL of 0.5× Steady-Glo (Promega, E2250). Maintain Steady-Glo at 4° C. and warm to room temperature using a Combi cassette with a 6-foot input line submerged in a room temperature water bath.

18) Relid plates and return to the Liconic Carousel. Incubate at room temperature for 30 min.

19) Delid plates and move to an Envision plate reader (Perkin Elmer). Collect luminescence values using with 100 ms read time per well. Relid the plates and discard.

### Screening Protocol 2: Primary ReTest in H4 Cells

As described in the primary screen in H4 cells with selected retest samples.

### Screening Protocol 3: Secondary Assay in H4-C Cells

As described in the primary retest in H4 cells but using the H4-C cell line.

### Screening Protocol 4: Secondary Assay in H4-PRP Cells

As described in the primary retest in H4 cells but using the H4-PRP cell line.

Screening Protocol 5: Western Blot Secondary Screen in H4 Cells Human H4 neuroglioblastoma cells were cultured in DMEM supplemented with 10% FBS and penicillin/streptomycin. Cells were exposed to increasing concentrations of compound (0, 0.1, 1, 10, 100 μM) for 48 hours. Cytoplasmic protein lysates were prepared by homogenizing the cells in midRIPA buffer (25 mM Tris pH 7.4, 1% NP40, 0.5% sodium deoxycholate, 15 mM NaCl, protease inhibitors, RNase inhibitor, and 10 mM DTT). Western blotting for alpha-synuclein was performed using mouse monoclonal anti-alpha-synuclein, and anti-beta-actin. The blots were developed using chemiluminescence (Pierce), visualized with a PhosphoImager (BioRad, Hercules, CA), and the bands were quantified using QuantityOne software (BioRad).

The specific protocol is as follows:

1) Culture human H4 neuroglioblastoma cells in Dulbeccos modified essential medium (Invitrogen) supplemented with 10% FBS (Invitrogen) and penicillin/streptomycin (Bio-Whittaker, Walkerville, MD).

2) Expose cells to increasing concentrations of compound (0, 0.1, 1, 10, 100 μM) for 48 hours.

3) Prepare cytoplasmic protein lysates by homogenizing the cells in midRIPA buffer (25 mM Tris pH 7.4, 1% NP40, 0.5% sodium deoxycholate, 15 mM NaCl, protease inhibitors, RNase inhibitor and 10 mM DTT).

4) Perform Western blotting for alpha-synuclein using mouse monoclonal anti-alpha-synuclein (BD Transduction Laboratories), and anti-beta-actin (Chemicon). Develop the blots using chemiluminescence (PIERCE) and visualize with a PhosphoImager (BioRad, Hercules, CA). Quantify the bands using QuantityOne software (BioRad).

### Screening Protocol 6: ELISA Secondary Screen in SH-SY5Y Neuroglioma Cells

A human ASYN ELISA kit was utilized for all ELISA experiments (Invitrogen alpha-synuclein ELISA kit KHB0061) and the kit protocol was followed.

1) Plate SH-SY5Y neuroglioblastoma cells (Corning, 3570) at 10,000 cells/well in 50 μL phenol red free media (phenol red-free DMEM with 4.5 g/L D-glucose [Lonza, 12-917F] supplemented with 10% FBS [Lonza, 14-503E], 200 μM L-glutamine [Lonza, 17-605E], 100 μM penstrep [Lonza, 17-602E] and 200 μg/mL geneticin [Invitrogen, 10131-027]).

2) Lid plates, then incubate cells for 24 hours at 37° C., 5% CO2, 95% humidity in a tissue culture incubator.

3) Pin 100 nL of each compound into the assay plates spanning a final concentration range from 100-0.006 μM.

4) Incubate plates for 48 h at 37° C., 5% CO2, 95% humidity. Following incubation, add 5 μL of Alamar blue solution per well and allow to incubate for 1 h at 37° C., 5% CO2, 95% humidity.

5) Read Alamar blue fluorescence using a Thermo Varioskan (570 nm excitation, 585 nm emission). Remove cell media and add 30 μL cell lysis solution with protease inhibitor (G Biosciences, 786-331; 10 μL/mL).

6) Place plates on a plate shaker for 1 h, before removing 20 μL lysate. Transfer cell lysates to 500 μL Eppendorf tubes and centrifuge (Eppendorf Centrifuge 5417C) for 10 min at 13,000 rpm.

7) Add 12 μL of the cell supernatant to 48 μL of standard diluent buffer (1:5). Add these samples to ELISA plate wells. Prepare ASYN standard curve wells as described in the kit manual.

8) Add 50 μL of anti-α-Synuclein (Detection Antibody) solution to each well, tapping the plate to mix. Cover the plate with a tape seal and incubate for 3 h at RT.

9) Wash wells four times with wash buffer using a multichannel pipette and add 100 μL of anti-rabbit IgG-HRP antibody solution to each well. Cover the plate and incubate for 30 min at RT. Wash wells four times with wash buffer and add 100 μL of Stabilized Chromogen to each well. Seal the plate and incubate in the dark at RT for 30 min.

10) Finally, add 100 μL of Stop Solution to each well, tapping the plate to mix, and read absorbance at 450 nm using an Envision plate reader (Perkin Elmer).

### General PET Imaging Procedures

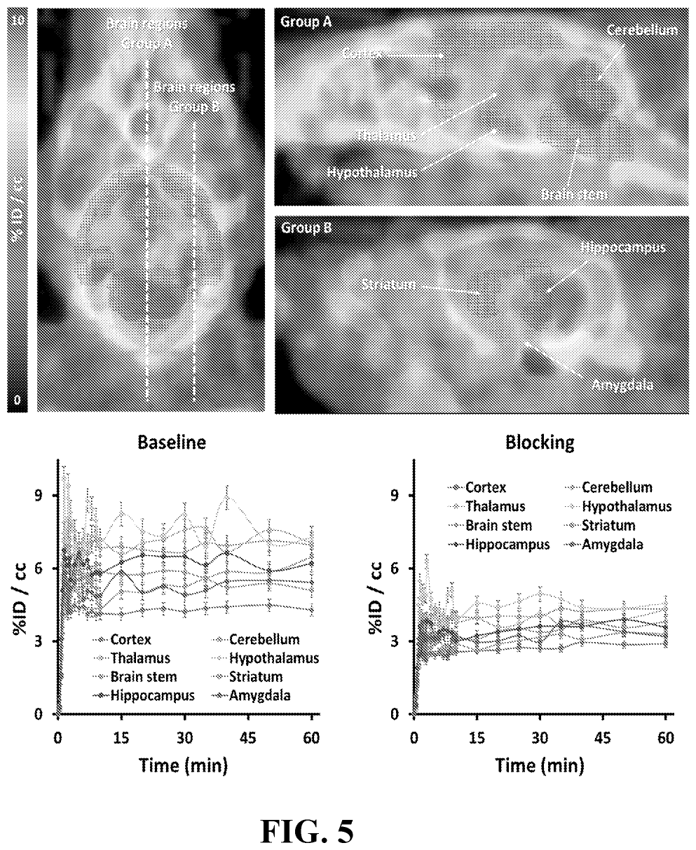

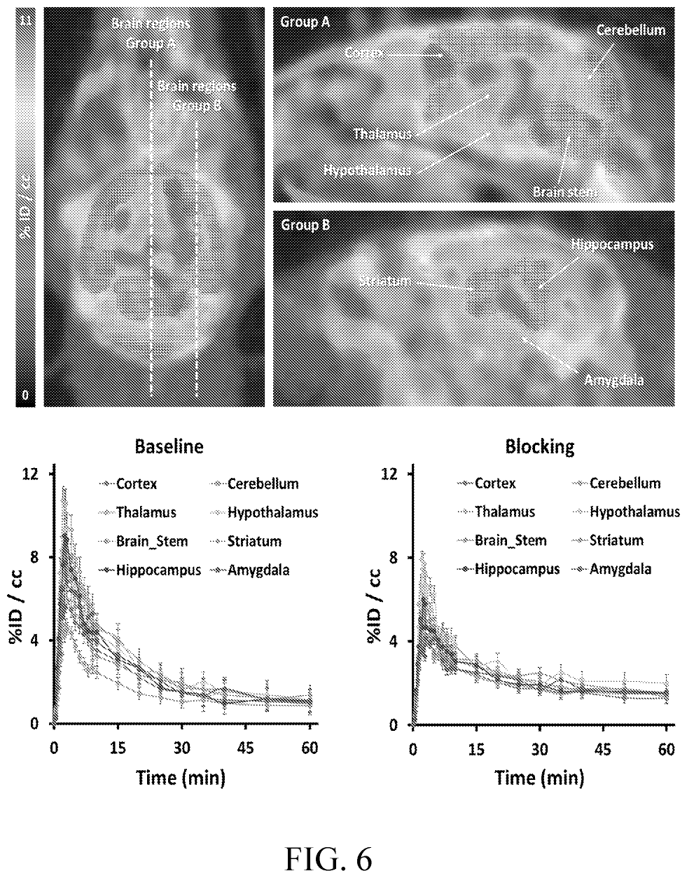

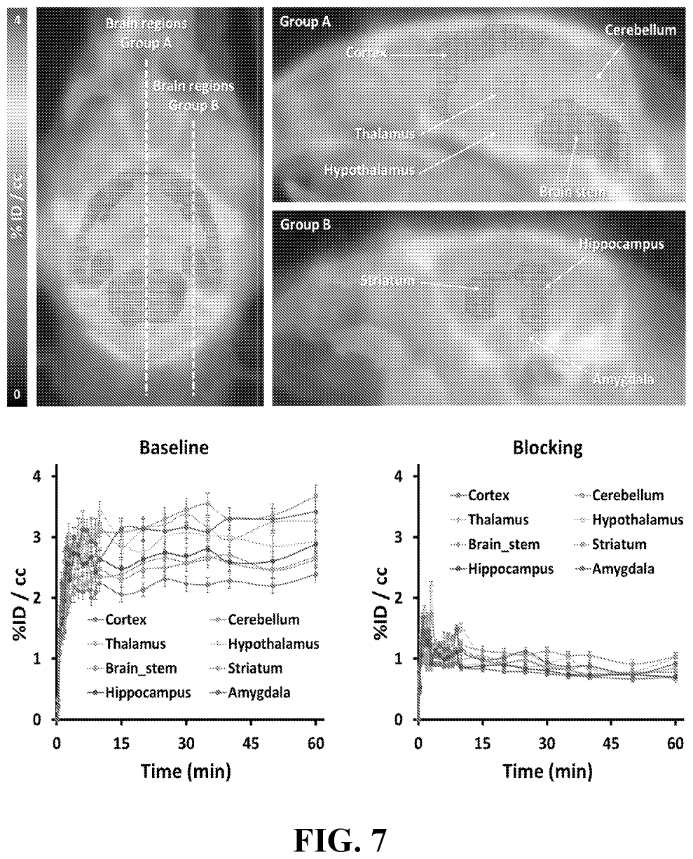

Rodent PET-CT Acquisition and Post Processing. Rodents were anesthetized with inhalational isoflurane and then arranged in a Triumph Trimodality PET/CT/SPECT scanner (Gamma Medica, Northridge, CA). For baseline study, radiotracers were administered at 0.1 mCi in 0.1 mL. For blocking studies, pretreat unlabeld compound was administered at 3 mg/mg, 5-min prior to radiotracer administration. Dynamic PET acquisition was 60 min and followed by CT for anatomic co-registration. PET data was reconstructed using a 3D-MLEM method and exported from the scanner in DICOM format with an anatomic CT. These files were imported to PMOD (PMOD Technologies, Ltd.). Rodent PET-CT Image Analysis. Volumes of interest (VOIs) were drawn manually as spheres in brain regions guided by high resolution CT structural images and summed PET data. Time-activity curves (TACs) were exported in terms of decay corrected activity per unit volume at specified time points with gradually increasing intervals and expressed as percent injected dose per unit volume for analysis. The results are shown in FIGS. 5, 6, and 7.

Peroxide Levels in DA Neurons Treated with Compound 10

Midbrain dopamine neurons from a patient treated with live cell dyes comparing conditions that increase or decrease iron levels in cells (Hemin and Deferiprone (DFP, Fe chelator)) to rule out potential off-target actions of compound 10 vs. posiphen to cause oxidative stress and ferroptosis.

Treatment of these neurons reduced lipid peroxides, reactive oxygen species cell death compared to controls similar to iron chelation with deferiprone (DFP), P<05. The numbers in Table 1 represent percent staining from fixed FACS analysis of single cells. Reactive oxygen species toxicity was significantly lowered by compound 10 with SD=1.5% for OXBODIPY stains, P=0.016 while SD=0.16%, P=0.0386 for CELLROX+stains (DFP with SD=2.23% for OXBODIPY, P=0.034, while DFP's SD=0.22% for CELLROX+, P=0.033).

TABLE 1
Midbrain dopamine neurons treated with live cell dyes

| | CON | HEMIN | DFP | 10 | POSIPHEN | |
|---|---|---|---|---|---|---|
| % OX-BODIPY- | 21.81 | 11.07 | 12.97 | 12.42 | 29.95 | LIPID PEROXIDATION |
| 488/CELLS | | | | | | |
| % CELL-ROX- | 26.92 | 24.9 | 14.26 | 15.27 | 28.05 | REACTIVE OXYGEN |
| 647/CELLS | | | | | | SPECIES |
| % Zombie- | 16.59 | 9.07 | 12.97 | 13.82 | 23.99 | APOPTOSIS |
| NIR/CELLS | | | | | | |
| % Annexin V- | 8.97 | 15.41 | 6.63 | 2.54 | 11.21 | FERROPTOSIS/ |
| 405/CELLS | | | | | | NECROPTOSIS |

TABLE 2
Compound properties summary

| | | | | Decrease | Decrease | Decrease |
|---|---|---|---|---|---|---|
| | IC₅₀/μM | | | αsyn(%) | αsyn(%) | αsyn(%) |
| Cmpd. | (SNCA | Selectivity | | SHSY5Y | DA | X3 SNCA |
| No. | 5′UTR) | PGL3 5′UTR PrP-5′UTR | | cells | neurons | neurons |
| 1 | 0.1 | 58-fold | 126-fold | 14-28 @ 1 | ND | ND |
| | | | | μM | | |
| 2 | 5.8 | 21-fold | 18-fold | 25-50 @ 0.1 | 60 @ 1 μM | 50 |
| | | | | μM | | |
| 3 | 0.134 | 31-fold | 17-fold | 15-19 @ 1-10 | 60 @ 1 μM | 50 |
| | | | | μM | | |
| 4 | 2.8 | 43-fold | 18-fold | 25-50 @ 1 | ND | ND |
| | | | | μM | | |
| 5 | 0.194 | 66-fold | Inc. | 20-50 @ 0.1 | ND | ND |
| | | | | μM | | |
| 6 | 0.1 | 80-fold | Inc. | 21-42 @ 0.1-1 | ND | ND |
| | | | | μM | | |
| 7 | 0.127 | 68-fold | 127-fold | 30-60 @ 1 | ND | ND |
| | | | | μM | | |
| 8 | 0.200 | 53-fold | 58-fold | 20-40 @ 0.1-1 | ND | ND |
| | | | | μM | | |
| 9 | 0.100 | 1,200-fold | ND | ND | 90 @ 1 μM | ND |
| 10 | 1.8-3.5 | 34-fold | 35-fold | 19-38 @ 0.1 | 60 @ 1 μM | 25 |
| | | | | μM | | |

| | | | | | |
|---|---|---|---|---|---|
| Posiphen | 10 | Mikkilineni et al. 2012 | 25-50 @10 | 25 | 5 |

μM

TABLE 3
Compound properties summary

| | IC₅₀/M | Selectivity | | | | Decrease syn(%) |
|---|---|---|---|---|---|---|
| | (SNCA | PGL3 5′UTR | | Decrease syn(%) | Decrease syn(%) | X3 SNCA neuron |
| Compounds | 5′UTR) | PrP-5′UTR | | SHSY5Y cells | DA neurons | Original name |
| 2 | 5.8 | 21-fold | 18-fold | 25-50 @ 0.1M | 60 @ 1M | 50 A3. CC #2 |
| 3 | 0.134 | 31-fold | 17-fold | 15-19 @ 1-10M | 60 @ 1M | 50 B1. CC#3 |
| 9 | 0.100 | 1,200-fold | ND | ND | 90 @ 1M | ND C1. CC#4 |
| 10 | 1.8-3.5 | 34-fold | 35-fold | 19-38 @ 0.1M | 60 @ 1M | 25 Syn-516. CC#1 |
| 11 | 0.1 | 58-fold | 126-fold | 14-28 @ 1M | ND | ND A6. CC #5 |
| 12 | 0.1 | 80-fold | Inc. | 21-42 @ 0.1-1M | ND | ND A4. CC#6 |
| 13 | 0.200 | 53-fold | 58-fold | 20-40 @ 0.1-1M | ND | ND A10. CC#7 |
| 14 | 0.194 | 66-fold | Inc. | 20-50 @ 0.1M | ND | ND A11. CC#8 |
| 15 | 2.8 | 43-fold | 18-fold | 25-50 @ 1M | ND | ND B3. CC#9 |
| 16 | 0.127 | 68-fold | 127-fold | 30-60 @ 1M | ND | ND B8. CC#10 |

| | | | | | |
|---|---|---|---|---|---|
| Posiphen | 10 | Mikkilineni | 25-50 @10M | 25 | 5 |
| | | et al. 2012 | | | |

### REFERENCES

1. Ueda K, et al., Proc Natl Acad Sci USA 1993 Dec. 1; 90:12282-12286.
2. Singleton A, et al., Lancet 2004 Sep. 25-Oct. 1; 364(9440):1105-1107.
3. Scherzer C R, et al., Proc Natl Acad Sci USA 2008 Aug. 5; 105(31): 10907-10912.
4. Cantuti-Castelvetri I, et al., J Neuropathol Exp Neurol 2005 December; 64(12):1058-1066.
5. Thomson A M, et al., Int J Biochem Cell Biol 1999 (October); 31(10):1139-1152.
6. Thomson A M, et al., J Biol Chem 2005 Aug. 26; 280(34):30032-30045.Epub 2005 Jun. 20.
7. Oru E E, et al., J Med Chem 2004 Dec. 30; 47(27):6760-6767.
8. Jiang H, et al., 2016. Mol Neurobiol doi:10.1007/s12035-016-9879-1.
9. Ross N T, et al., 2010. Reports from the NIH Molecular Libraries Program. Bethesda (MD).
10. Singleton A, et al., 2004. Lancet 364: 1105-7.
11. Chartier-Harlin M C, et al., 2004. Lancet 364: 1167-9.
12. Ahn T B, et al., 2008. Neurology 70: 43-9.
13. Ikeuchi T, et al., 2008. Arch Neurol 65: 514-9.
14. Singleton A B, et al., 2003. Science 302: 841.
15. Kuo Y M, et al., 2019. Am J Neurodegener Dis 8: 1-15.
16. Chiba-Falek O, et al., 2006. Mov Disord. 21, 10, 1703-1708.
17. Lewis J, et al., 2008. Mol Neurodegener 3: 19.
18. Teich A F, et al., Alzheimers Dement (NY) 4: 37-45.
19. Kuo Y M, et al., 2010. Hum Mol Genet 19: 1633-50.
20. Taguchi T, et al., 2020. Brain 143: 249-65.
21. Mikkilineni S, et al., 2012. Parkinsons Dis 2012: 142372.
22. Rogers J T, et al., 2011. J Neural Transm 118: 493-507.
23. Zhang P, et al., 2020. Proc Natl Acad Sci USA 117: 1457-67.
24. Friedlich A L, et al., 2007. Mol Psychiatry 12: 222-3.
25. Rogers J T, et al., 2016. J Neurochem 138: 479-94.
26. Thomson A M, et al., 2005. J Biol Chem 280: 30032-45.
27. Rogers J T, et al., 2004. Curr Drug Targets 5: 535-51.
28. Wang W, et al., 2007. J Biol Chem 282: 24650-9.
29. Zhang F, et al., 2008. J Biol Chem. 2008; 283(49):33911-8.
30. Olivares d, et al., 2009. Int J Mol Sci 10: 1226-60.
31. Cahill C M, et al., 2008. Biochim Biophys Acta.
32. Cho H H, et al., 2010. J Biol Chem 285: 31217-32.
33. Rogers J T, et al., 2002. J Biol Chem 277: 45518-28.
34. Bandyopadhyay S, et al., 2006. J Neural Transm: 237-47.
35. Fields C R, et al., 2019. Front Mol Neurosci 12: 299.
36. Bakshi R, et al., 2019. Ann Neurol 85: 593-9.
37. Posturna R, et al., 2019. Movement Disorders 34: 665-75.
38. Peres T V, et al., 2016. BMC Pharmacol Toxicol 17: 57.
39. Harischandra D S, et al., 2019. Sci Signal 12. 572.
40. Venkataramani V, et al., 2018. J Neurochem 147: 831-48.
41. Youdim M B, et al., 1994. Life Sci 55: 2077-82.
42. Trudler D, et al., 2014. J Neurochem 129: 434-47.
43. Youdim M B, et al., 2001. Cell Mol Neurobiol 21: 555-73.
44. Swalley S E, et al., 2020. Bioorg Med Chem 28: 115239.
45. Ueda K, et al., 1993. Proc Natl Acad Sci USA 90: 11282-6.
46. Lee V M, et al., 2004. Trends Neurosci 27: 129-34.
47. Qureshi M M, et al., 2008. The open Neurology Journal 2: 51-4.
48. Taylor J P, et al., 2006. Trends Mol Med 12: 76-82.
49. Cahill C M, et al., 2008. J Biol Chem 283: 25900-12.
50. Oakley A E, et al., 2007. Neurology 68: 1820-5.
51. Jensen P H, et al., 1997. Biochem J 323 (Pt 2): 539-46.
52. Conway K A, et al., 1998. Nat Med 4: 1318-20.
53. Han H, et al., 1995. Chem Biol 2: 163-9.
54. Trojanowski J Q, et al., 2001. Parkinsonism Relat Disord 7: 247-51.
55. Cantuti-Castelvetri I, et al., 2005. J Neuropathol Exp Neurol 64: 1058-66.
56. Lahiri D K, et al., 2007. J Pharmacol Exp Ther 320: 386-96.
57. Farlow M, et al., 2004. Expert Rev Neurother 4: 799-808.
58. Shaw K T, et al., 2001. Proc Natl Acad Sci USA 98: 7605-10.
59. Venti A, et al., 2004. Ann N Y Acad Sci 1035: 34-48.
60. Utsuki T, et al., 2007. J Pharmacol Exp Ther 321: 353-61.
61. Ross N T, et al., 2010. Probe Reports from the NIH Molecular Libraries Program. Bethesda (MD).
62. Ross N T, et al., 2010. Probe Reports from the NIH Molecular Libraries Program. Bethesda (MD).
63. Tiong S H, et al., 2015. Fitoterapia 102: 182-8.
64. Tiong S H, et al., 2013. Don. Molecules 18: 9770-84.
65. Ross N T, et al., 2011. Alarcon-Aris D, 2018. Mol Ther 26: 550-67.
66. Jankovic J, et al., 2018. JAMA Neurol 75: 1206-14.
67. Schenk D B, et al., 2017. Mov Disord 32: 211-8.
68. Levin J, et al., 2014. Acta Neuropathol 127: 779-80.
69. Mao X, et al., 2016. Science 353.
70. Finkelstein D I, et al., 2017. Acta Neuropathol Commun 5: 53.
71. Tam D H, et al., 2006. Am J Hematol 81: 443-7.
72. Gao J, et al., 2018. Neurotherapeutics 15: 156-75.
73. Taylor, et al., 2019. Brain Behav Immun.
74. Cabin D E, et al., 2005. Neurobiol Aging 26: 25-35.
75. Melrose H L, et al., 2006. Exp Brain Res 173: 196-204.
76. Rogers J, 1987. PNAS 84: 2277-81.
77. Lok J, et al., 2012. Transl Stroke Res 3 Suppl 1: S119-24.
78. Cai W, et al., 2018. Mol Neurodegener 8: 44.
79. Ross, Probe Report of the Molecular Libraries Network of the NIH, pathway to discovery. 2011.
80. Mikkilineni, S, et al., Parkinson's disease 2012: 142372.Epub 2012.
81. Neystat M, et al., Movement Disorders, 1999, 14, 417-422.
82. Kim S, et al., Parkinsonism & Related Disorders, 2004, 10, supplement 1, S9-S13.
83. Kingsbury A, et al., Movement Disorders, 2004, 19: 162-170.
84. Tan E, et al., Movement Disorders, 2005, 20: 620-623.
85. Westerlund M, et al., FASEB Journal, 2008, 22: 3509-3514.
86. Fuchs J, et al., FASEB Journal, 2008, 22: 1327-1334.
87. Beyer K, et al., Journal of the Neurological Sciences, 2011, 310: 112-117.
88. Dumitriu A, et al., Parkinson's Disease, 2012, Article ID 614212.
89. Singleton A, et al., Lancet 2004, 364:1105-1107.
90. Chartier-Harlin M C, et al., Lancet 2004, 364:1167-1169.
91. Ahn T B, et al., Neurology 2008, 70:43-49.
92. Ikeuchi T, et al., Arch Neurol 2008, 65:514-519.
93. Donahue C P, et al., J Biomol Screen. 2007 September; 12(6):789-99.
94. Kim T W, et al., Cell Stem Cell. 2021 Feb. 4; 28(2):343-355.e5.

What is claimed is:

1. A method for treating an α-synuclein associated disease or disorder in a subject in need thereof, comprising administering to the subject a therapeutically effective amount of a compound of selected from the group consisting of: 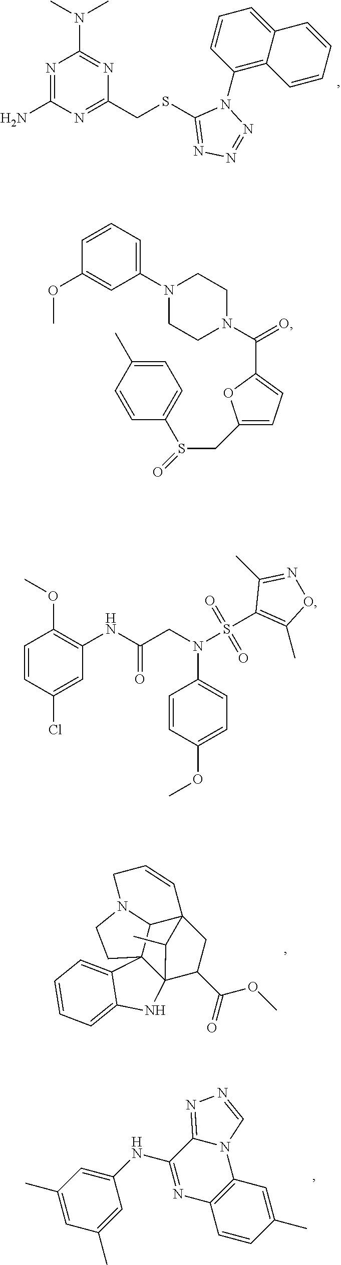 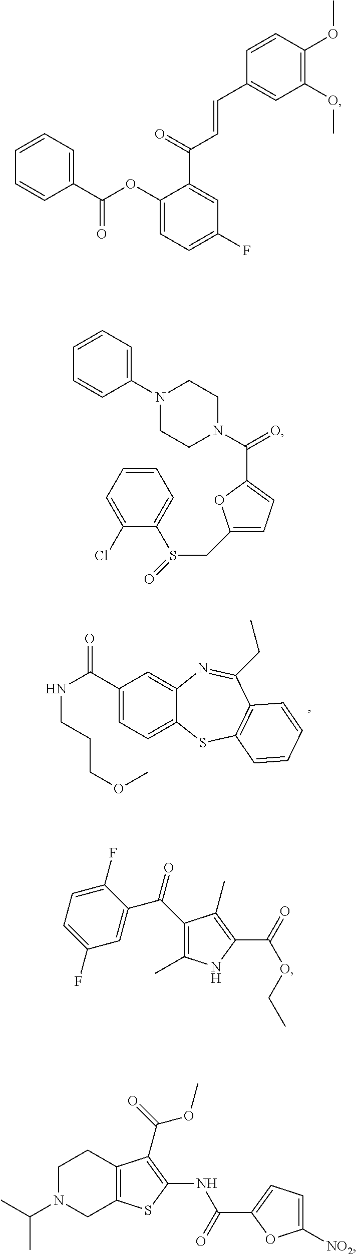 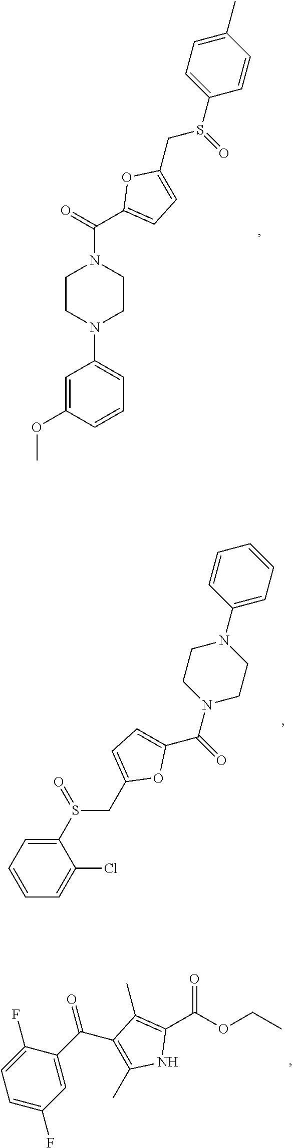 [SMILES: COc1cccc(N2CCN(C(=O)c3ccc(CS(=O)c4ccc(C)cc4)o3)CC2)c1 ; O=C(c1ccc(CS(=O)c2ccccc2Cl)o1)N1CCN(c2ccccc2)CC1 ; CCOC(=O)c1[nH]c(C)c(C(=O)c2cc(F)ccc2F)c1C] 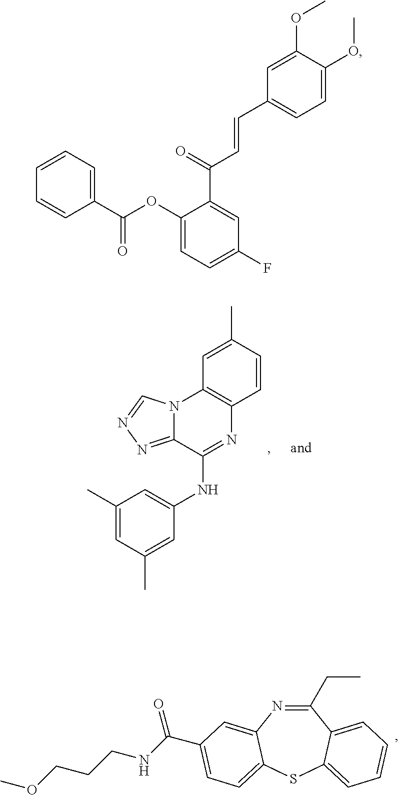
or a pharmaceutically acceptable salt of any of the foregoing.

2. A method for treating an α-synuclein associated disease or disorder in a subject in need thereof, comprising administering to the subject a therapeutically effective amount of a compound of selected from the group consisting of: 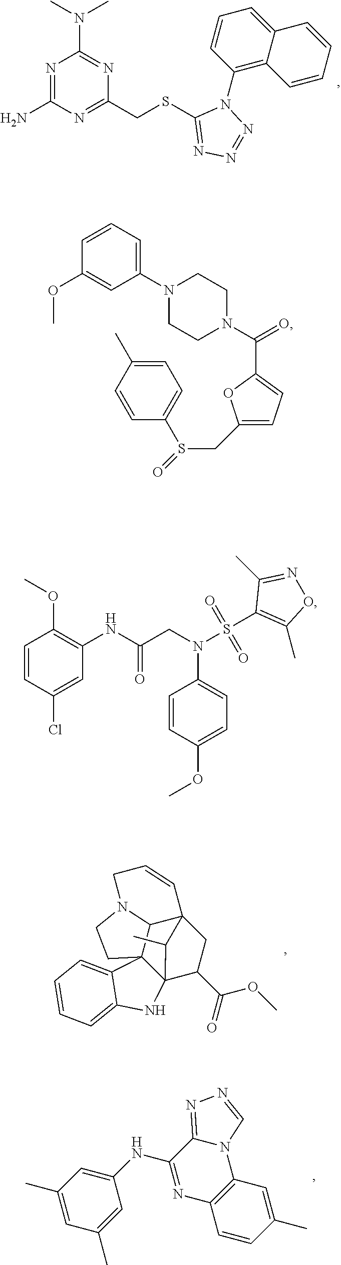 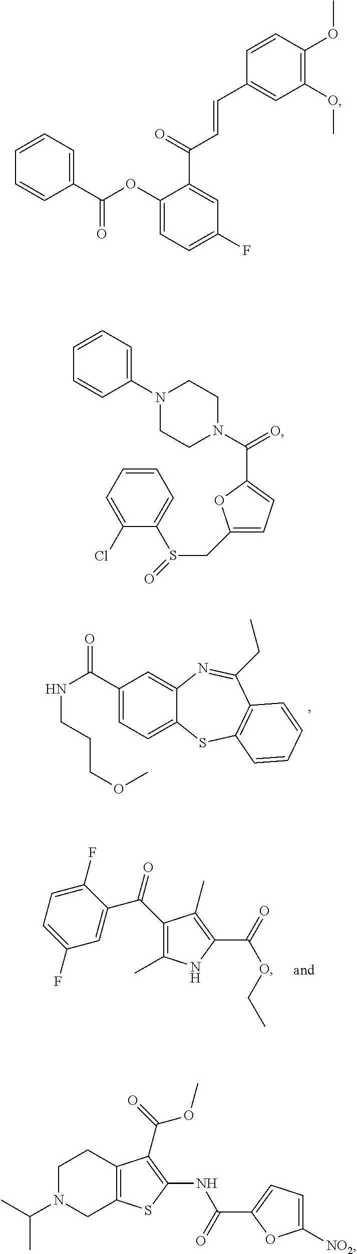
or a pharmaceutically acceptable salt of any of the foregoing.

3. The method of claim 1 or 2, wherein the -synuclein associated disease or disorder is Parkinson's disease.

4. The method of claim 1 or 2, wherein the -synuclein associated disease or disorder is dementia with Lewy bodies.

5. The method of claim 1 or 2, wherein the -synuclein associated disease or disorder is multiple systems atrophy.

6. The method of claim 1 or 2, wherein the -synuclein associated disease or disorder is prodromal REM sleep behavior disorder (RBD).

7. A method of treating an α-synuclein associated disease or disorder in a subject, comprising administering to a subject identified or diagnosed as having an α-synuclein associated disease or disorder a therapeutically effective amount of a compound selected from the group consisting of: 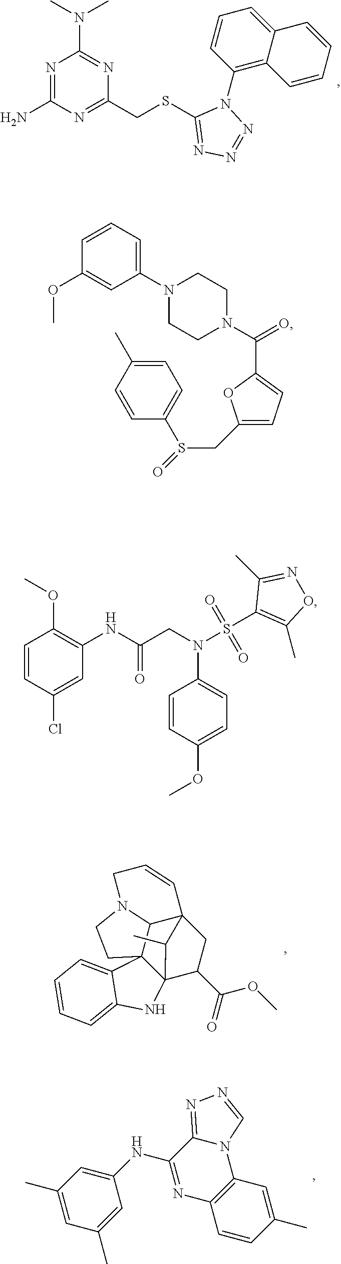 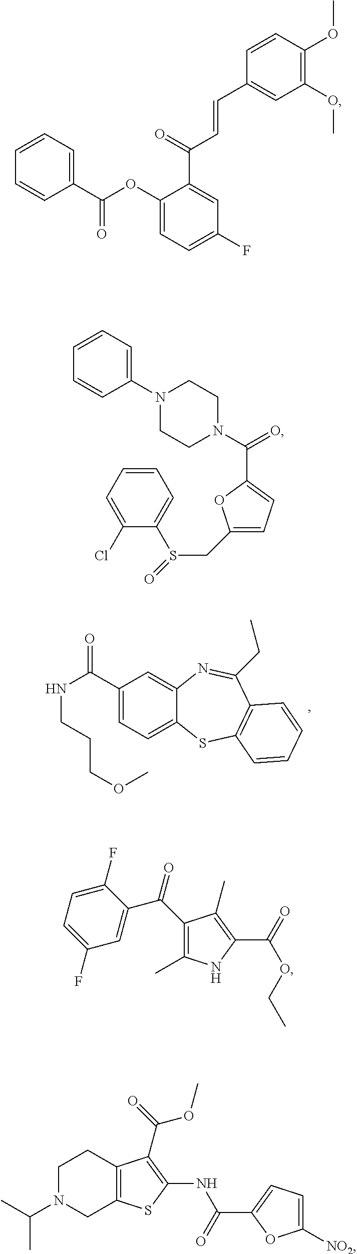 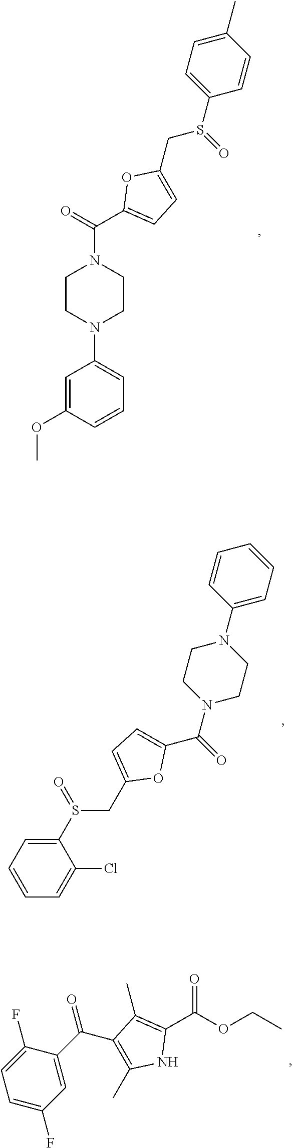 [SMILES: COc1cccc(N2CCN(C(=O)c3ccc(CS(=O)c4ccc(C)cc4)o3)CC2)c1 ; O=C(c1ccc(CS(=O)c2ccccc2Cl)o1)N1CCN(c2ccccc2)CC1 ; CCOC(=O)c1[nH]c(C)c(C(=O)c2cc(F)ccc2F)c1C] 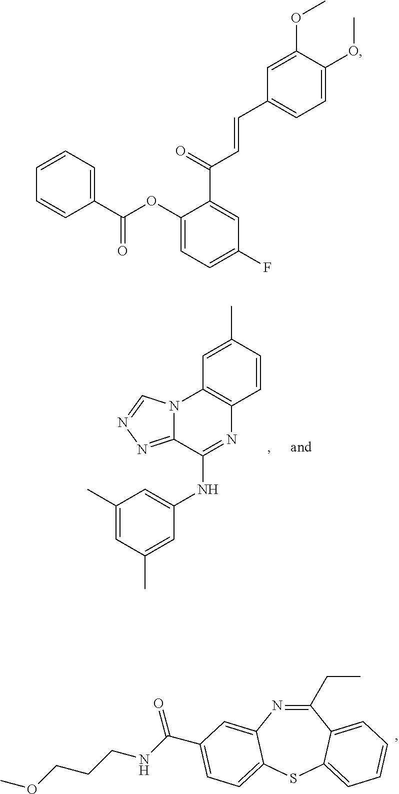
or a pharmaceutically acceptable salt of any of the foregoing.

8. A method of treating an α-synuclein associated disease or disorder in a subject, comprising administering to a subject identified or diagnosed as having an α-synuclein associated disease or disorder a therapeutically effective amount of a compound selected from the group consisting of: 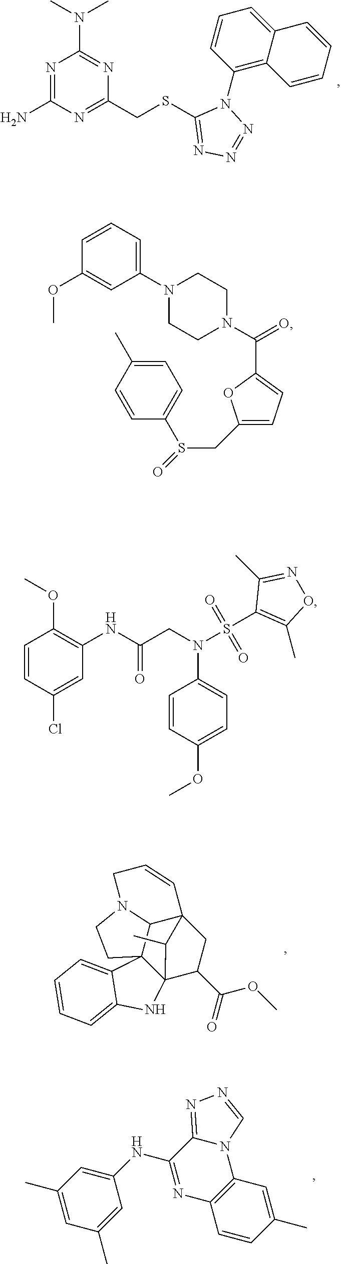 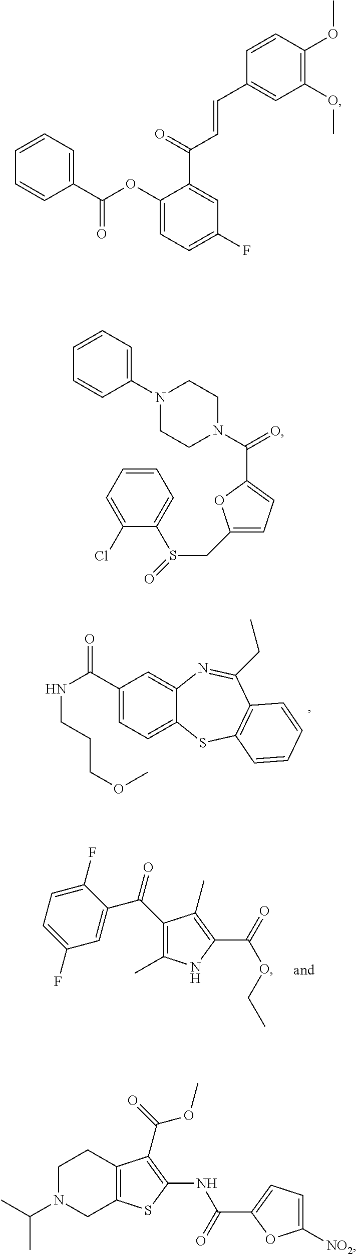
or a pharmaceutically acceptable salt of any of the foregoing.

9. A method of treating an α-synuclein associated disease or disorder in a subject, comprising: determining that the disease or disorder in the subject is an α-synuclein associated disease or disorder; and administering to the subject a therapeutically effective amount of a compound selected from the group consisting of: 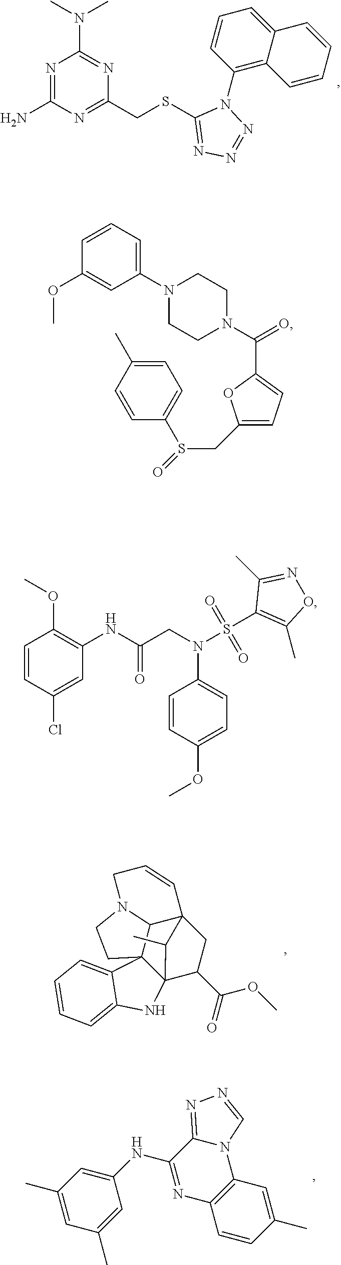 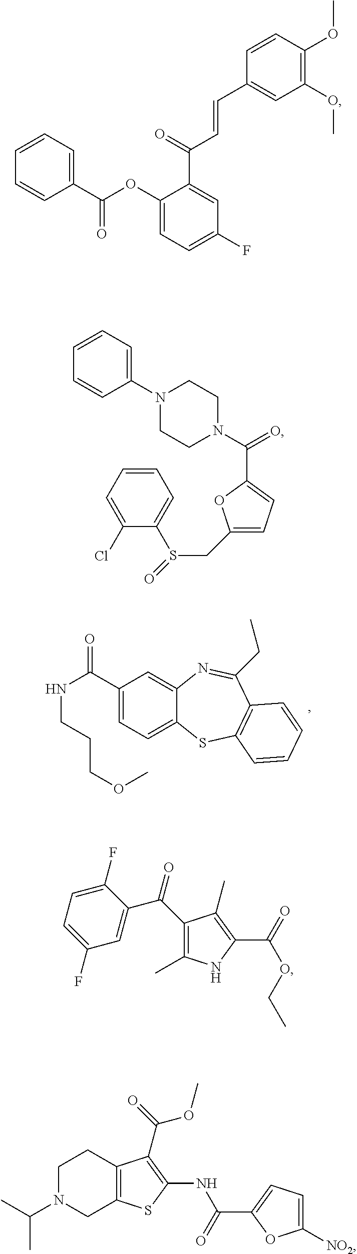 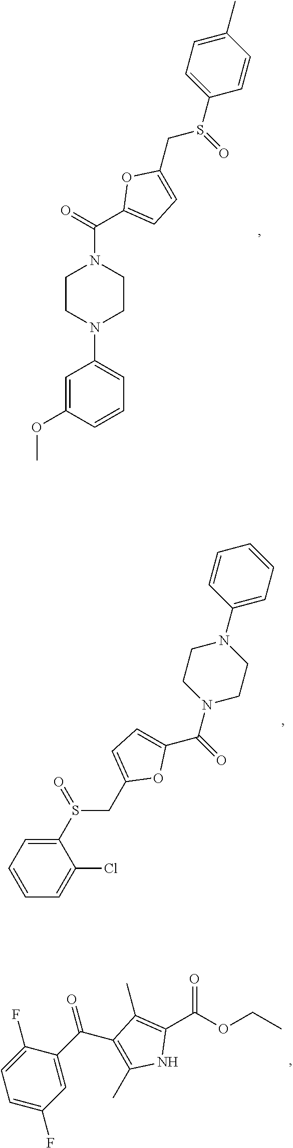 [SMILES: COc1cccc(N2CCN(C(=O)c3ccc(CS(=O)c4ccc(C)cc4)o3)CC2)c1 ; O=C(c1ccc(CS(=O)c2ccccc2Cl)o1)N1CCN(c2ccccc2)CC1 ; CCOC(=O)c1[nH]c(C)c(C(=O)c2cc(F)ccc2F)c1C] 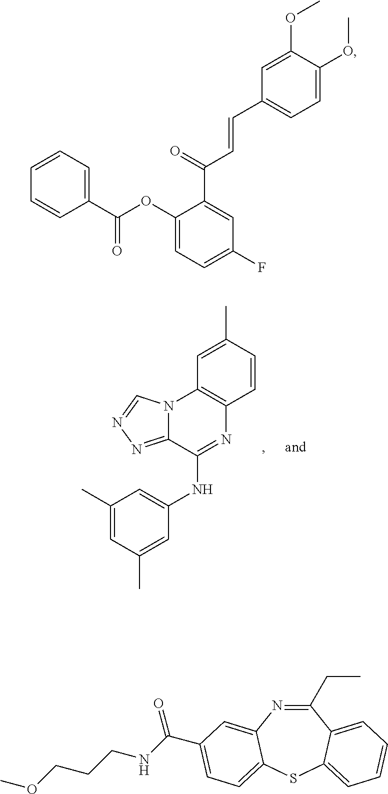
or a pharmaceutically acceptable salt of any of the foregoing.

10. A method of treating an α-synuclein associated disease or disorder in a subject, comprising: determining that the disease or disorder in the subject is an α-synuclein associated disease or disorder; and administering to the subject a therapeutically effective amount of a compound selected from the group consisting of: 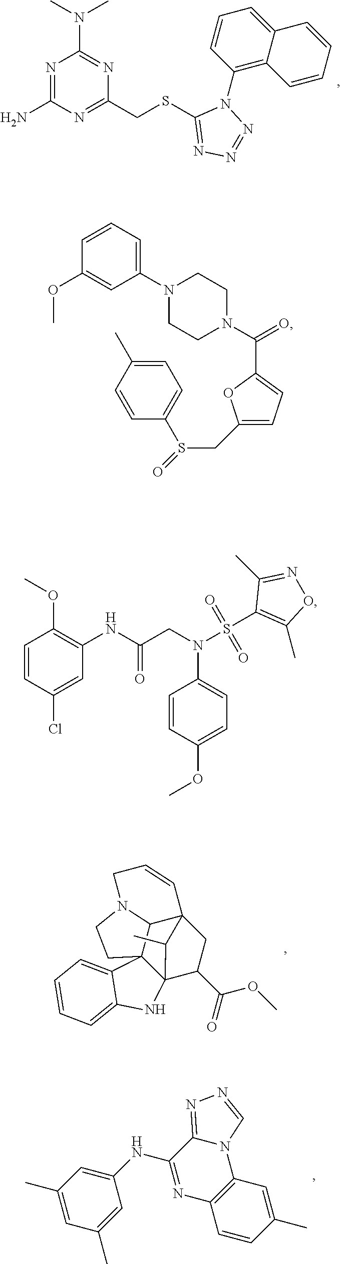 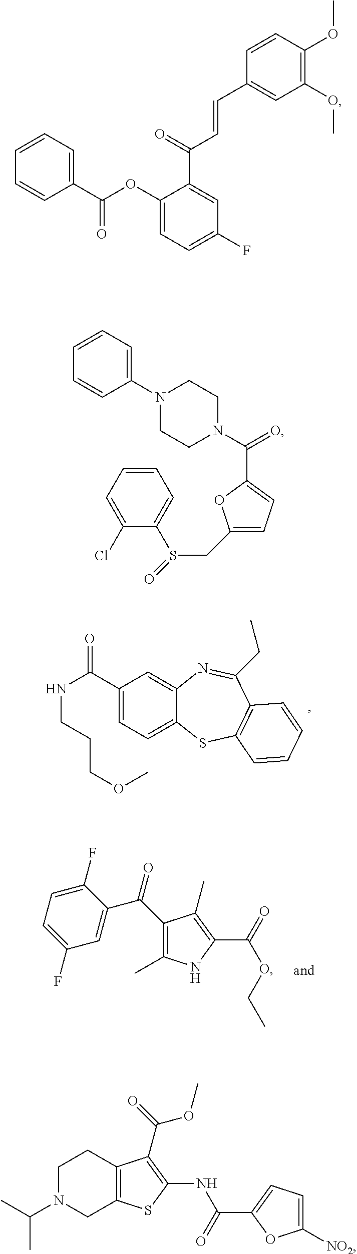
or a pharmaceutically acceptable salt of any of the foregoing.

11. The method of claim 9 or 10, wherein the determining that the disease or disorder in the subject is an α-synuclein associated disease or disorder comprises a genetic test.

12. The method of claim 9 or 10, wherein the determining that the disease or disorder in the subject is an α-synuclein associated disease or disorder comprises diagnostic imaging.

13. The method of claim 9 or 10, wherein the determining that the disease or disorder in the subject is an α-synuclein associated disease or disorder comprises neurological examination.

14. A method of treating a subject, comprising administering a therapeutically effective amount of a compound selected from the group consisting of: 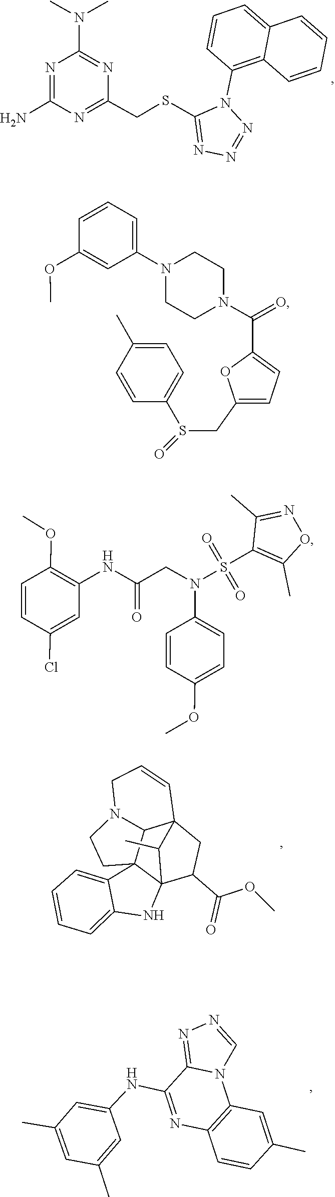 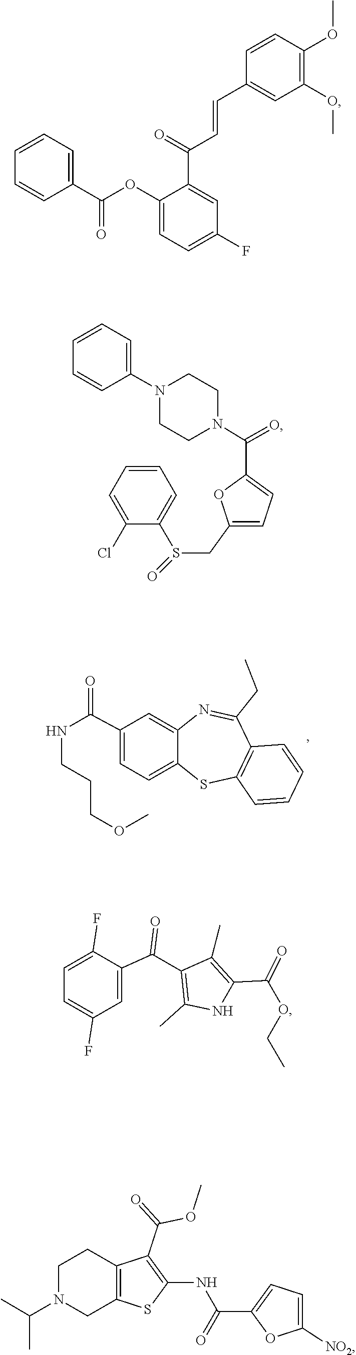 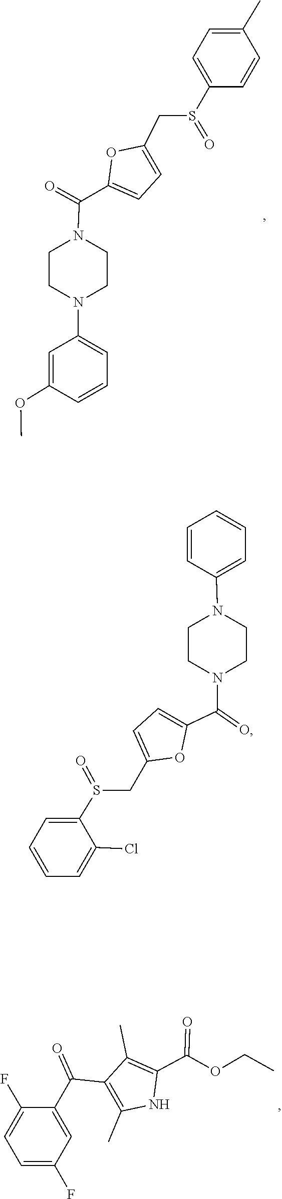 [SMILES: COc1cccc(N2CCN(C(=O)c3ccc(CS(=O)c4ccc(C)cc4)o3)CC2)c1 ; O=C(c1ccc(CS(=O)c2ccccc2Cl)o1)N1CCN(c2ccccc2)CC1 ; CCOC(=O)c1[nH]c(C)c(C(=O)c2cc(F)ccc2F)c1C] 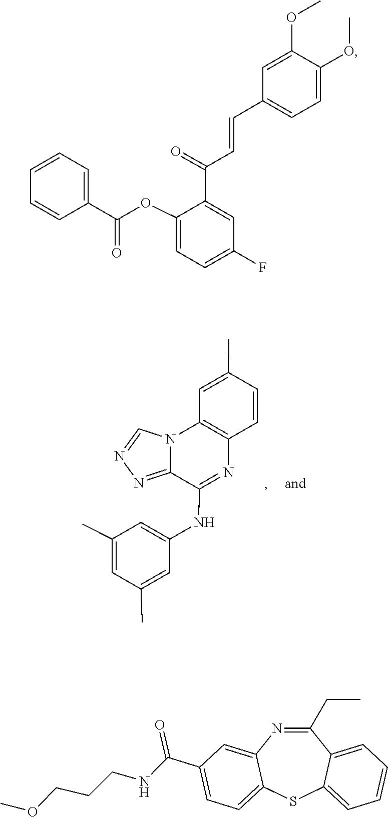
or a pharmaceutically acceptable salt of any of the foregoing, to a subject having a clinical record that indicates that the subject has an α-synuclein associated disease or disorder.

15. A method of treating a subject, comprising administering a therapeutically effective amount of a compound selected from the group consisting of: 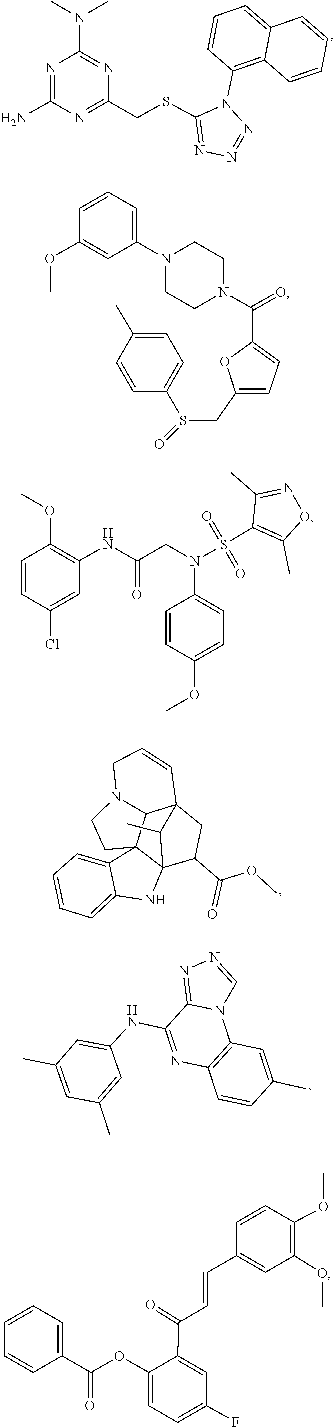 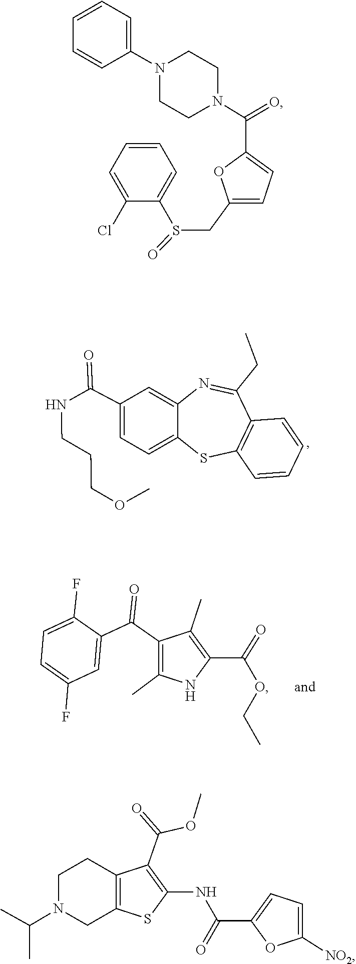 [SMILES: O=C(c1ccc(CS(=O)c2ccccc2Cl)o1)N1CCN(c2ccccc2)CC1 ; CCC1=Nc2cc(C(=O)NCCCOC)ccc2Sc2ccccc21 ; CCOC(=O)c1[nH]c(C)c(C(=O)c2cc(F)ccc2F)c1C ; COC(=O)c1c(NC(=O)c2ccc([N+](=O)[O-])o2)sc2c1CCN(C(C)C)C2]
or a pharmaceutically acceptable salt of any of the foregoing, to a subject having a clinical record that indicates that the subject has an α-synuclein associated disease or disorder.

16. A method for inhibiting α-synuclein translation in a mammalian cell, comprising contacting the mammalian cell with an effective amount of a compound selected from the group consisting of: 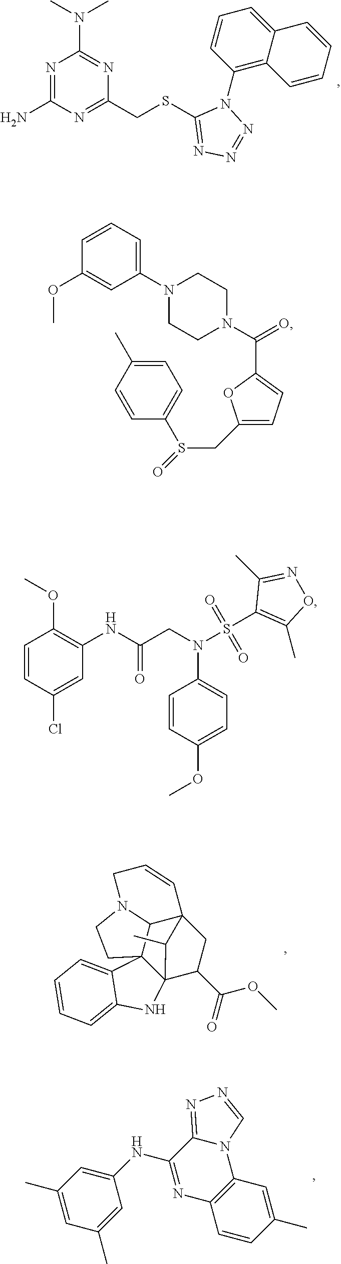 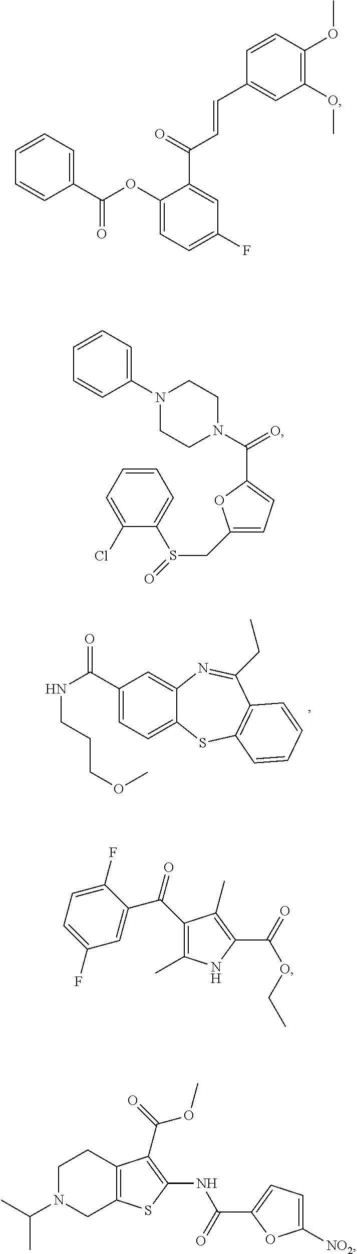 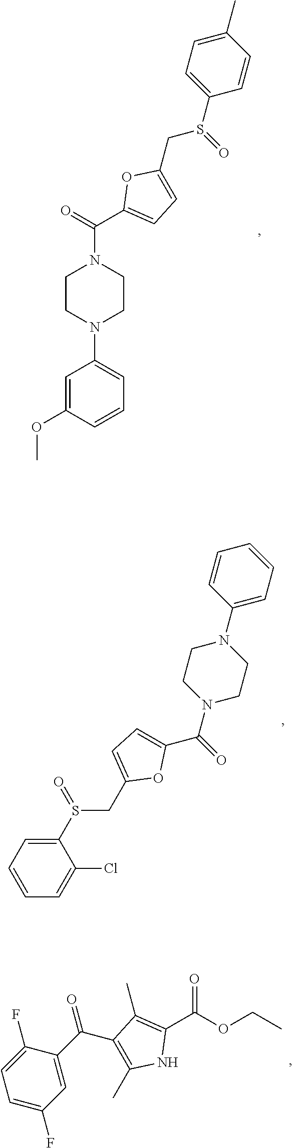 [SMILES: COc1cccc(N2CCN(C(=O)c3ccc(CS(=O)c4ccc(C)cc4)o3)CC2)c1 ; O=C(c1ccc(CS(=O)c2ccccc2Cl)o1)N1CCN(c2ccccc2)CC1 ; CCOC(=O)c1[nH]c(C)c(C(=O)c2cc(F)ccc2F)c1C] 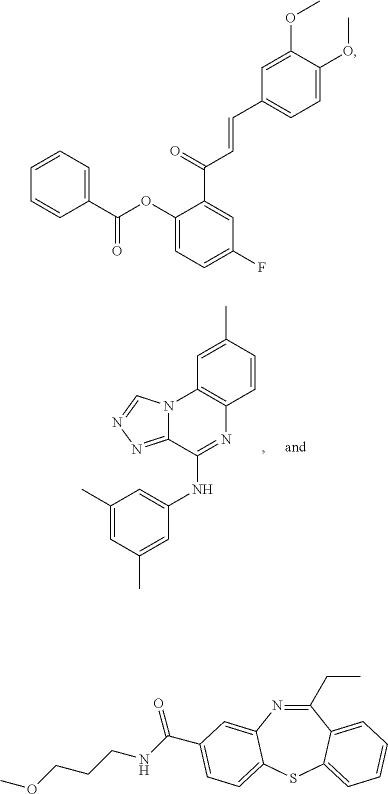
or a pharmaceutically acceptable salt of any of the foregoing.

17. A method for inhibiting α-synuclein translation in a mammalian cell, comprising contacting the mammalian cell with an effective amount of a compound selected from the group consisting of: 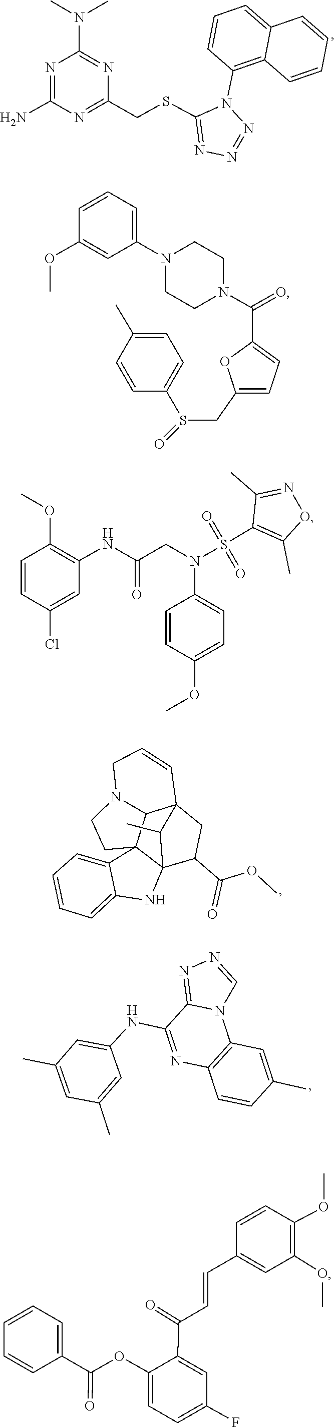 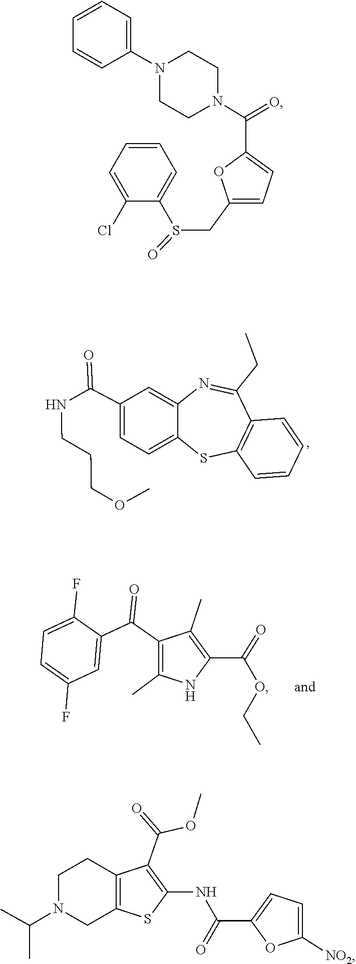 [SMILES: O=C(c1ccc(CS(=O)c2ccccc2Cl)o1)N1CCN(c2ccccc2)CC1 ; CCC1=Nc2cc(C(=O)NCCCOC)ccc2Sc2ccccc21 ; CCOC(=O)c1[nH]c(C)c(C(=O)c2cc(F)ccc2F)c1C ; COC(=O)c1c(NC(=O)c2ccc([N+](=O)[O-])o2)sc2c1CCN(C(C)C)C2]
or a pharmaceutically acceptable salt of any of the foregoing.

18. A method for inhibiting Lewy body formation in mammalian brain tissue, comprising contacting the mammalian brain tissue with an effective amount of a compound selected from the group consisting of: 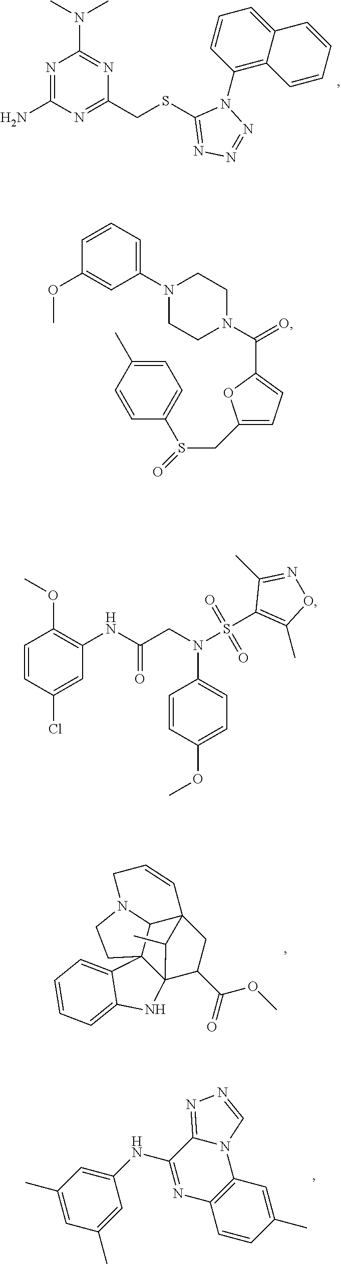 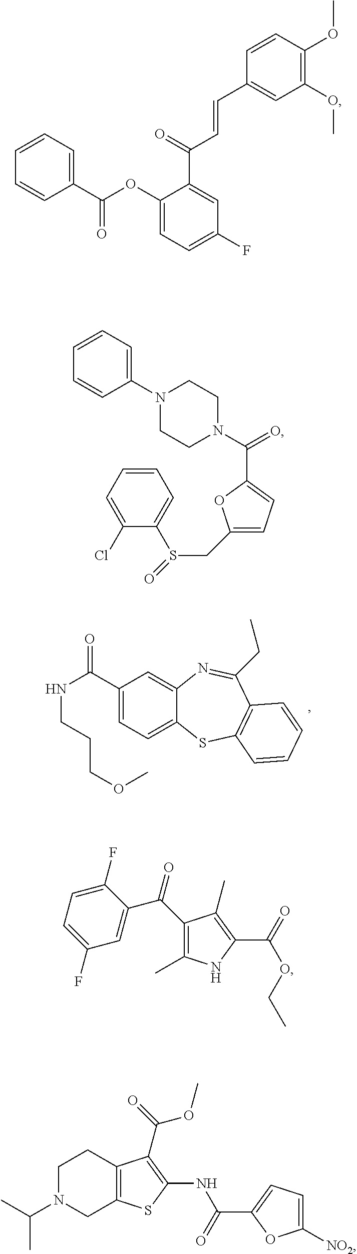 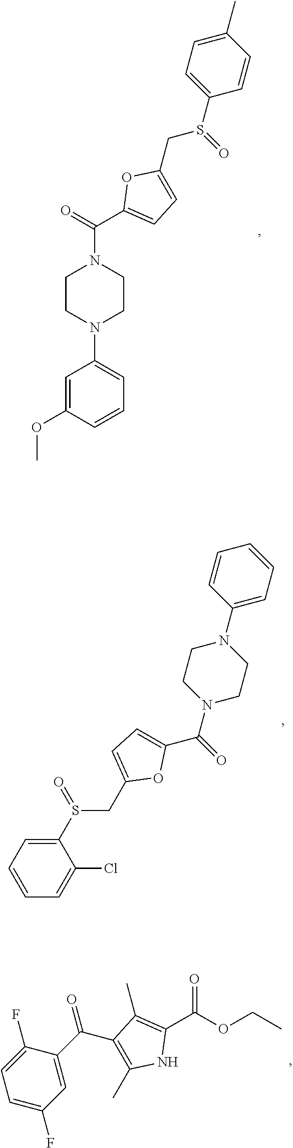 [SMILES: COc1cccc(N2CCN(C(=O)c3ccc(CS(=O)c4ccc(C)cc4)o3)CC2)c1 ; O=C(c1ccc(CS(=O)c2ccccc2Cl)o1)N1CCN(c2ccccc2)CC1 ; CCOC(=O)c1[nH]c(C)c(C(=O)c2cc(F)ccc2F)c1C] 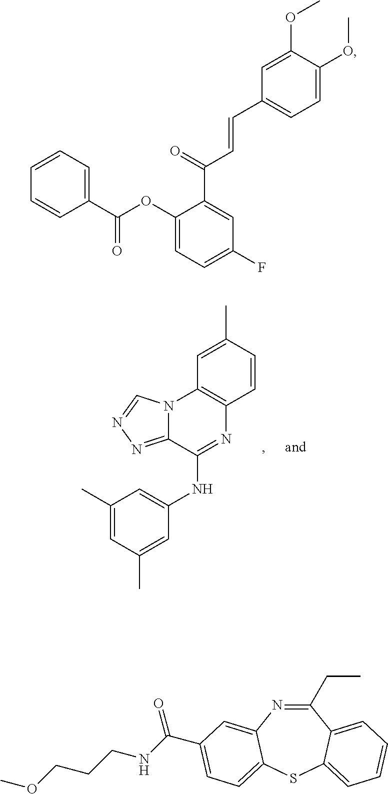
or a pharmaceutically acceptable salt of any of the foregoing.

19. A method for inhibiting Lewy body formation in mammalian brain tissue, comprising contacting the mammalian brain tissue with an effective amount of a compound selected from the group consisting of: 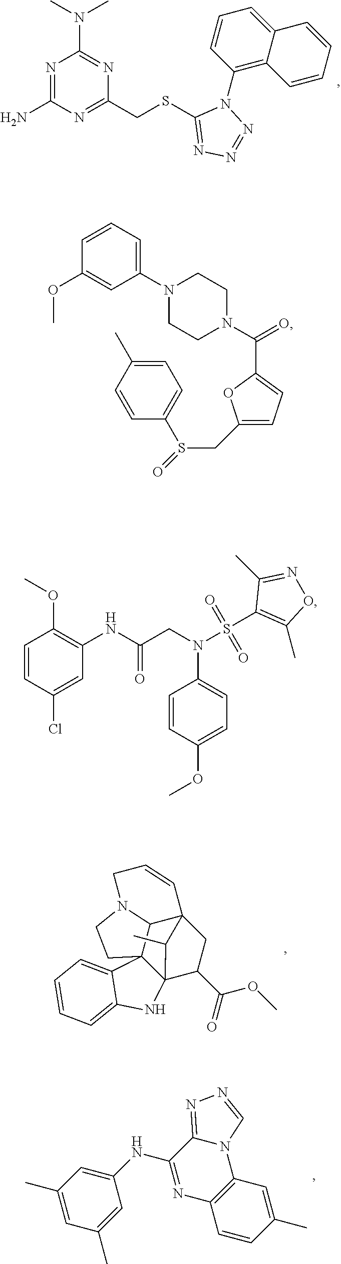 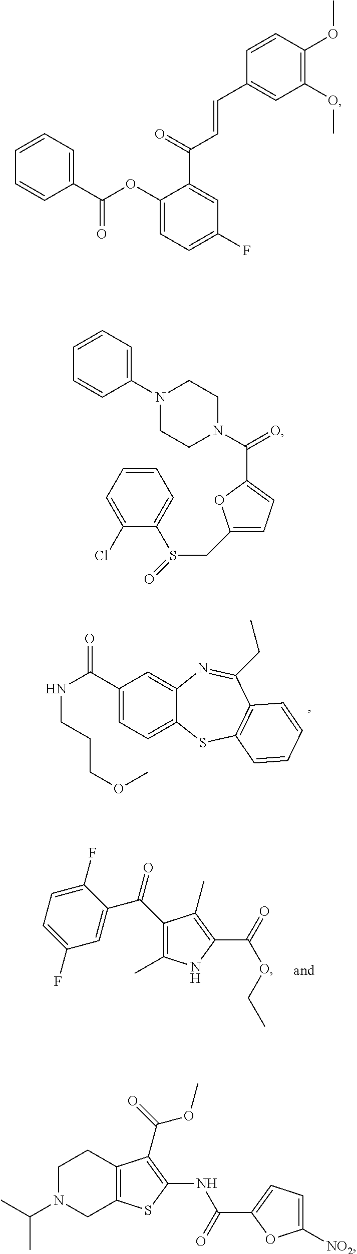
or a pharmaceutically acceptable salt of any of the foregoing.

20. The method of any one of claims 16-18, wherein the contacting is in vitro.

21. The method of any one of claims 16-18, wherein the contacting is in vivo.

22. The method of any one of claims 1-19, wherein the compound, or a pharmaceutically acceptable salt thereof, is selected from the group consisting of: 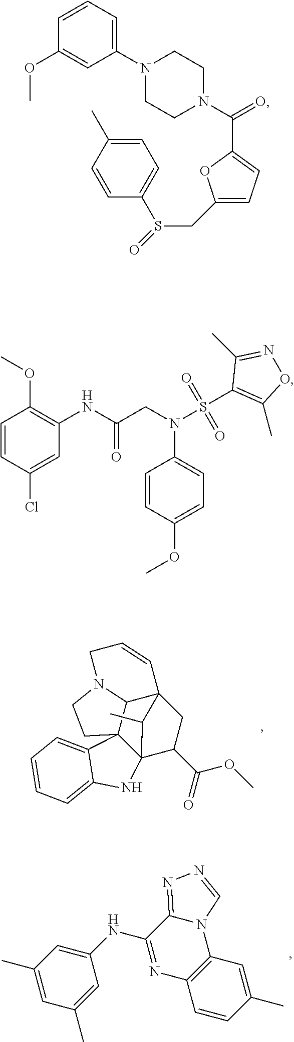 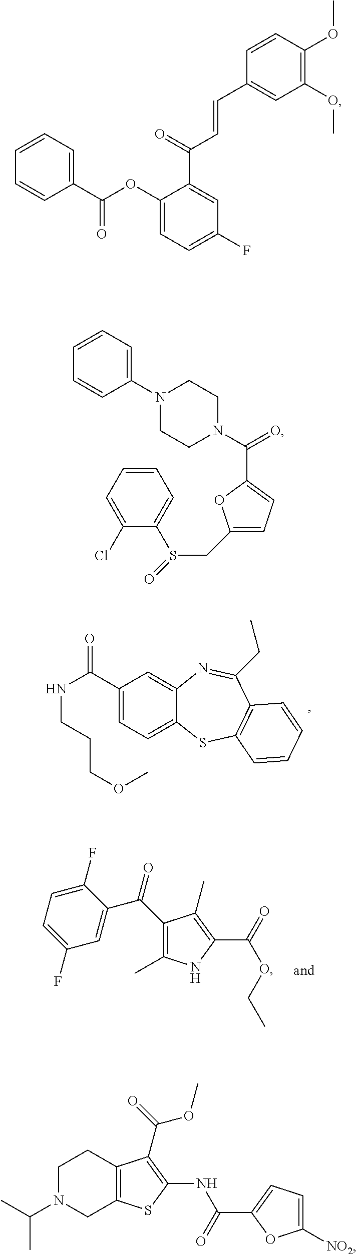
or a pharmaceutically acceptable salt of any of the foregoing.

23. A pharmaceutical composition comprising a compound selected from the group consisting of: 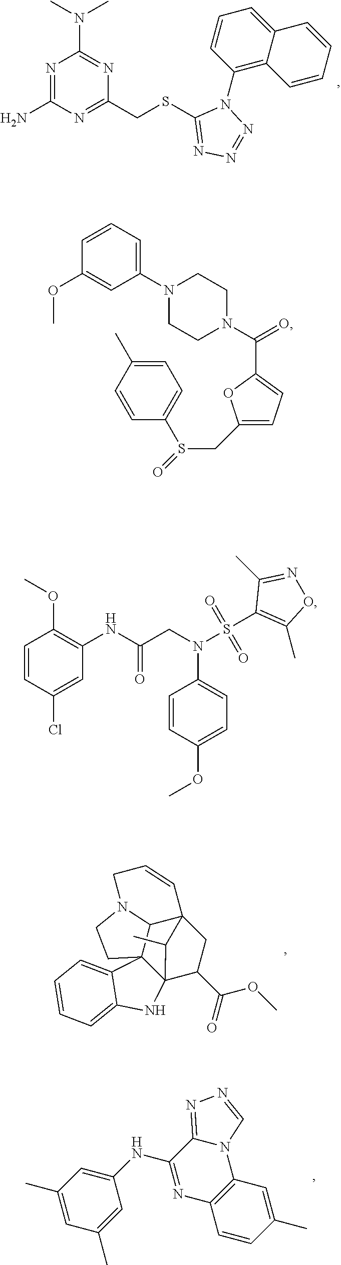 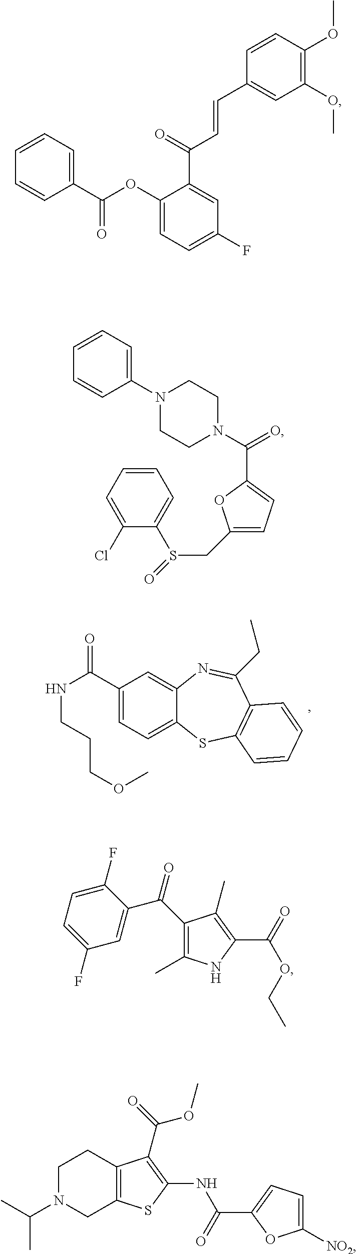 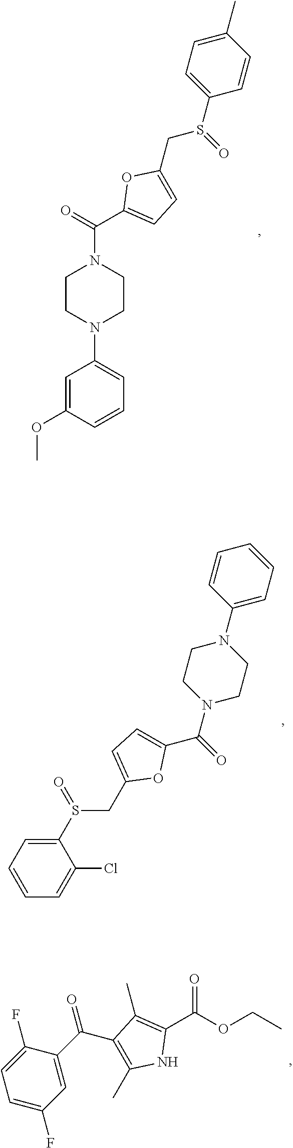 [SMILES: COc1cccc(N2CCN(C(=O)c3ccc(CS(=O)c4ccc(C)cc4)o3)CC2)c1 ; O=C(c1ccc(CS(=O)c2ccccc2Cl)o1)N1CCN(c2ccccc2)CC1 ; CCOC(=O)c1[nH]c(C)c(C(=O)c2cc(F)ccc2F)c1C] 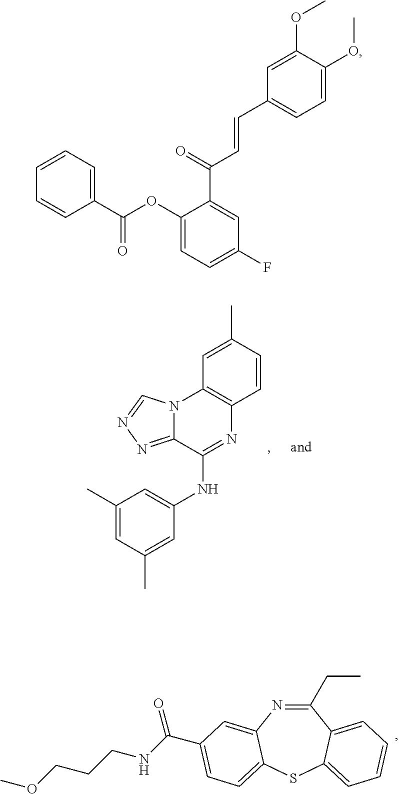
or a pharmaceutically acceptable salt of any of the foregoing, and one or more pharmaceutically acceptable excipients.

24. A pharmaceutical composition comprising a compound selected from the group consisting of: 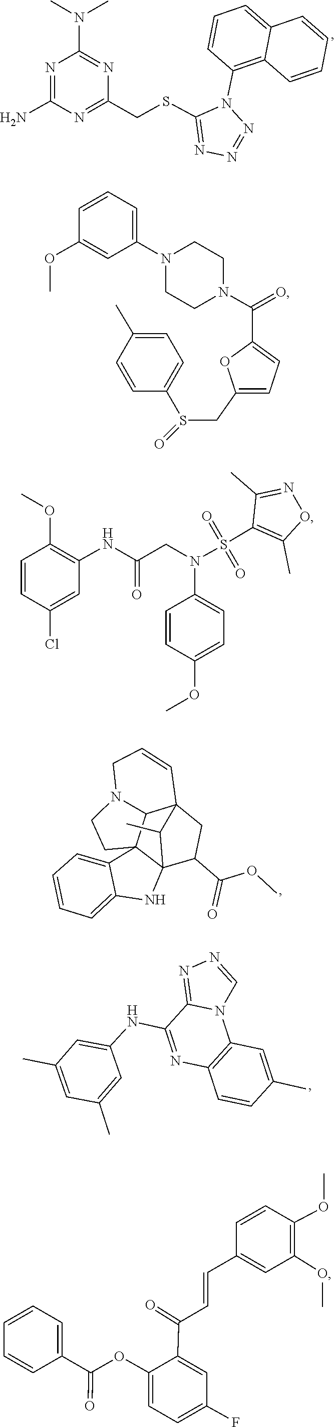 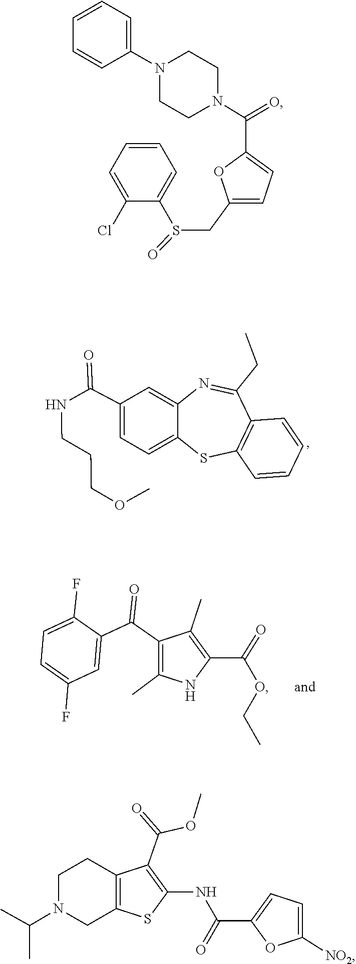 [SMILES: O=C(c1ccc(CS(=O)c2ccccc2Cl)o1)N1CCN(c2ccccc2)CC1 ; CCC1=Nc2cc(C(=O)NCCCOC)ccc2Sc2ccccc21 ; CCOC(=O)c1[nH]c(C)c(C(=O)c2cc(F)ccc2F)c1C ; COC(=O)c1c(NC(=O)c2ccc([N+](=O)[O-])o2)sc2c1CCN(C(C)C)C2]
or a pharmaceutically acceptable salt of any of the foregoing, and one or more pharmaceutically acceptable excipients.

25. The pharmaceutical composition of claim 24, wherein the compound, or a pharmaceutically acceptable salt thereof, is selected from the group consisting of: 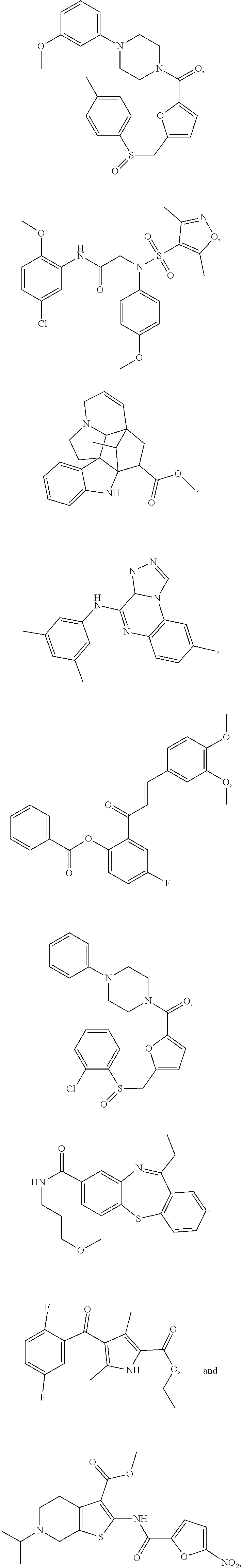
or a pharmaceutically acceptable salt of any of the foregoing.

26. A method for detecting an α-synuclein associated disease or disorder in a subject in need thereof, comprising administering to the subject an effective amount of a compound selected from the group consisting of: 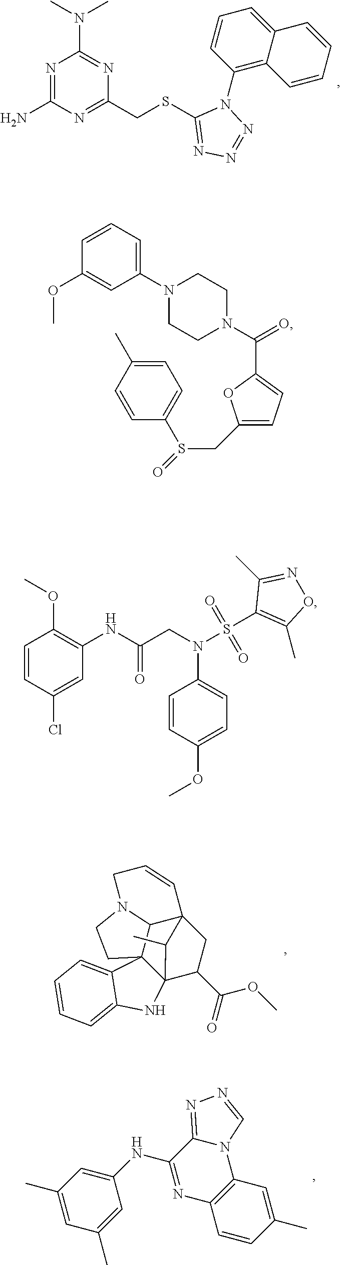 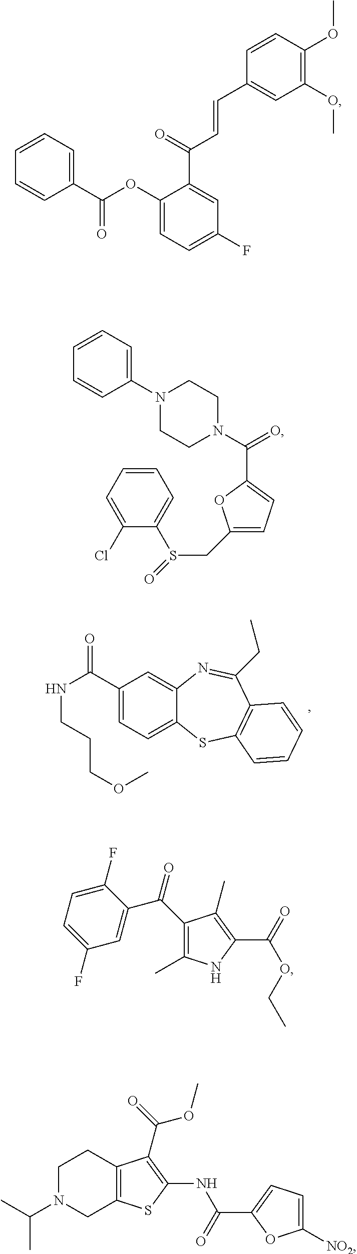 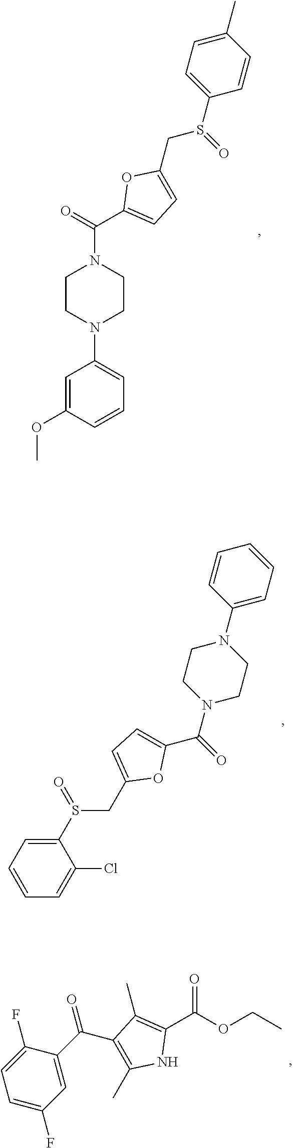 [SMILES: COc1cccc(N2CCN(C(=O)c3ccc(CS(=O)c4ccc(C)cc4)o3)CC2)c1 ; O=C(c1ccc(CS(=O)c2ccccc2Cl)o1)N1CCN(c2ccccc2)CC1 ; CCOC(=O)c1[nH]c(C)c(C(=O)c2cc(F)ccc2F)c1C] 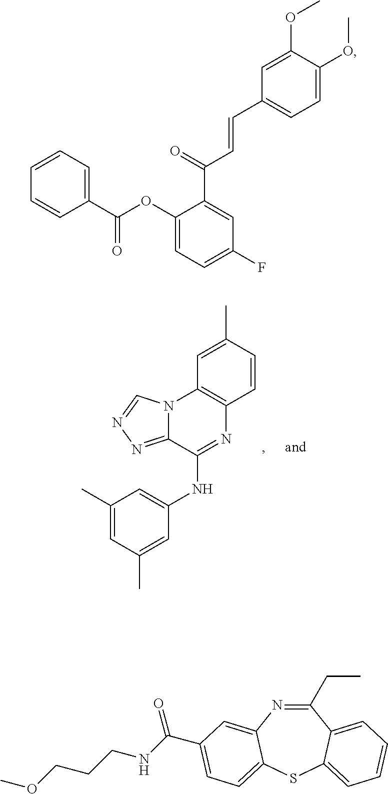
or a pharmaceutically acceptable salt of any of the foregoing; wherein the compound, or a pharmaceutically acceptable salt thereof, is radiolabeled.

27. A method for detecting an α-synuclein associated disease or disorder in a subject in need thereof, comprising administering to the subject an effective amount of a compound selected from the group consisting of: 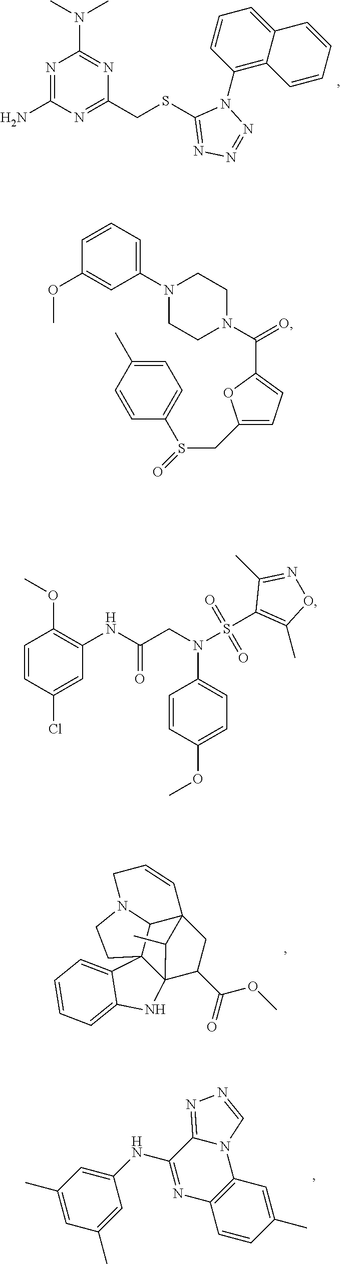 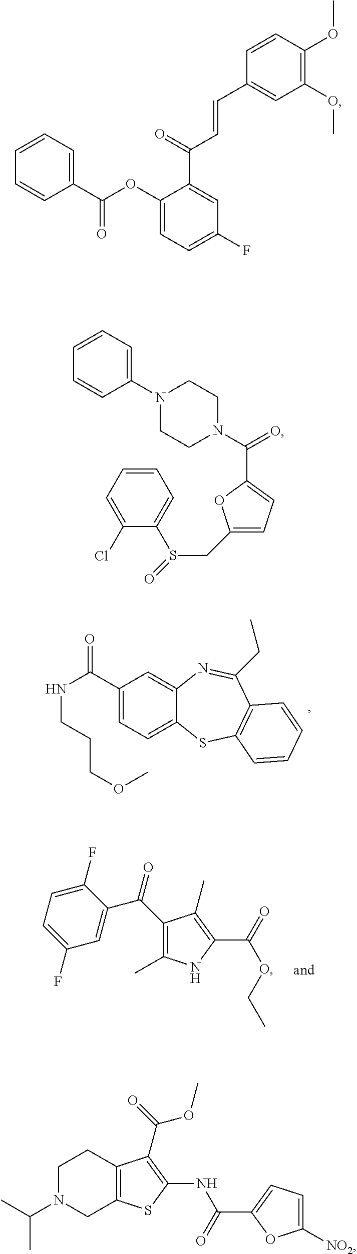
or a pharmaceutically acceptable salt of any of the foregoing; wherein the compound, or a pharmaceutically acceptable salt thereof, is radiolabeled.

28. The method of claim 27, wherein the compound, or a pharmaceutically acceptable salt thereof, is selected from the group consisting of: 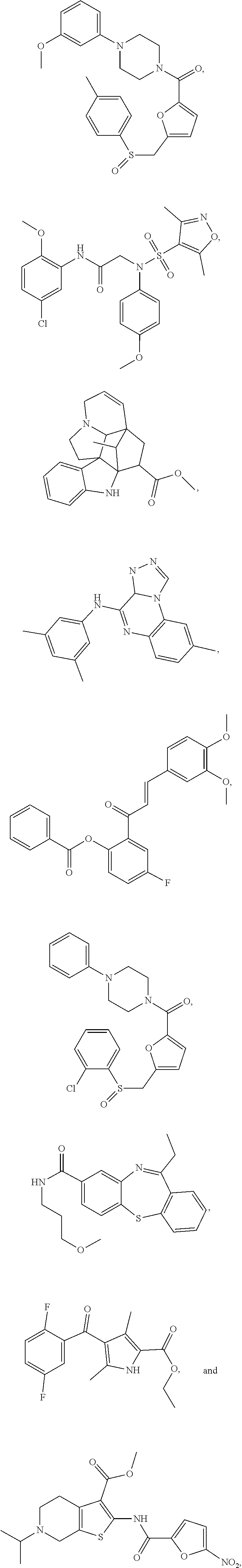
or a pharmaceutically acceptable salt of any of the foregoing.

29. The method of claim 27 or 28, wherein the radiolabeled compound is 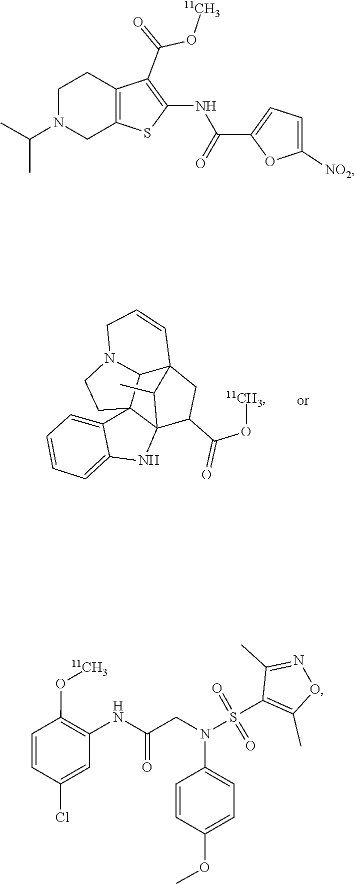
or a pharmaceutically acceptable salt of any of the foregoing.

30. The method of any one of claims 26-29, wherein the α-synuclein associated disease or disorder is Parkinson's disease.

31. The method of any one of claims 26-29, wherein the α-synuclein associated disease or disorder is Lewy bodies.

32. The method of any one of claims 26-29, wherein the α-synuclein associated disease or disorder is multiple systems atrophy.

33. The method of any one of claims 26-29, wherein the α-synuclein associated disease or disorder is prodromal REM sleep behavior disorder (RBD).

34. A method of detecting an α-synuclein associated disease or disorder in a subject, comprising administering to a subject suspected as having an α-synuclein associated disease or disorder an effective amount a compound selected from the group consisting of: 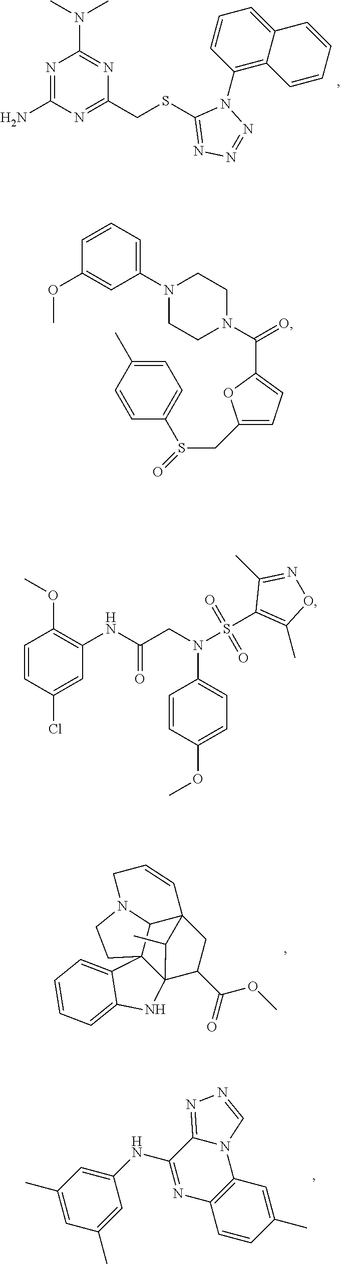 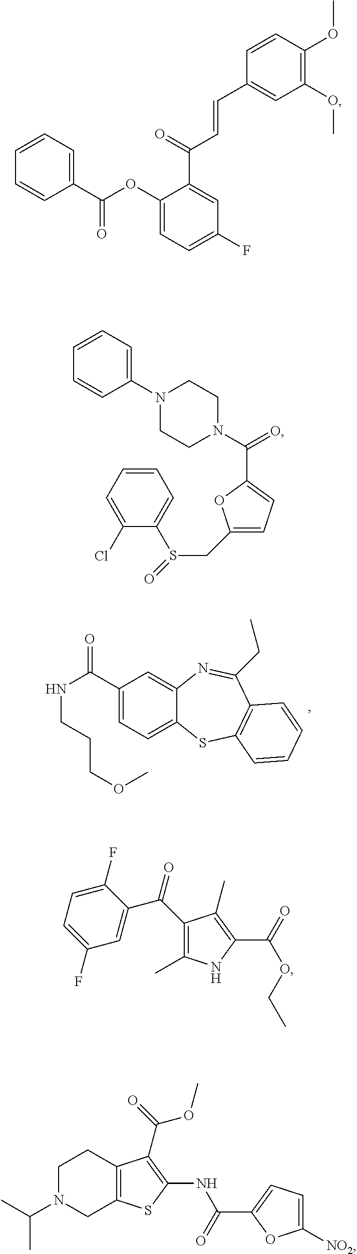 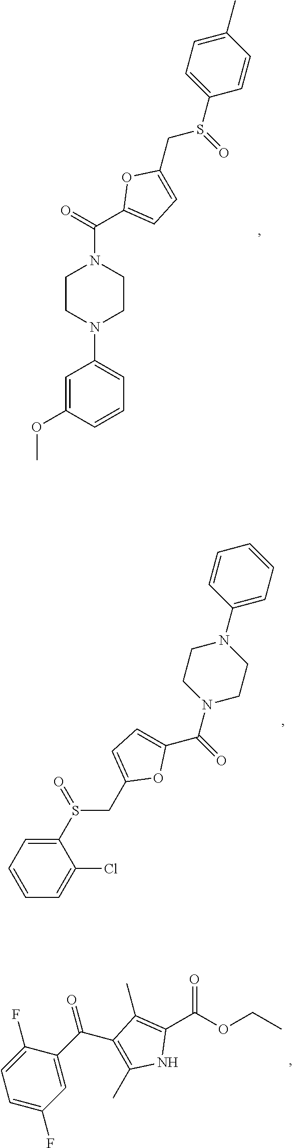 [SMILES: COc1cccc(N2CCN(C(=O)c3ccc(CS(=O)c4ccc(C)cc4)o3)CC2)c1 ; O=C(c1ccc(CS(=O)c2ccccc2Cl)o1)N1CCN(c2ccccc2)CC1 ; CCOC(=O)c1[nH]c(C)c(C(=O)c2cc(F)ccc2F)c1C] 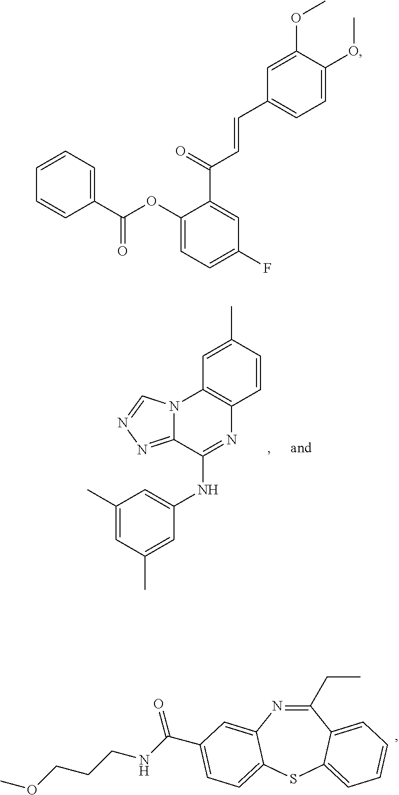
or a pharmaceutically acceptable salt of any of the foregoing; wherein the compound, or a pharmaceutically acceptable salt thereof, is radiolabeled.

35. A method of detecting an α-synuclein associated disease or disorder in a subject, comprising administering to a subject suspected as having an α-synuclein associated disease or disorder an effective amount a compound selected from the group consisting of: 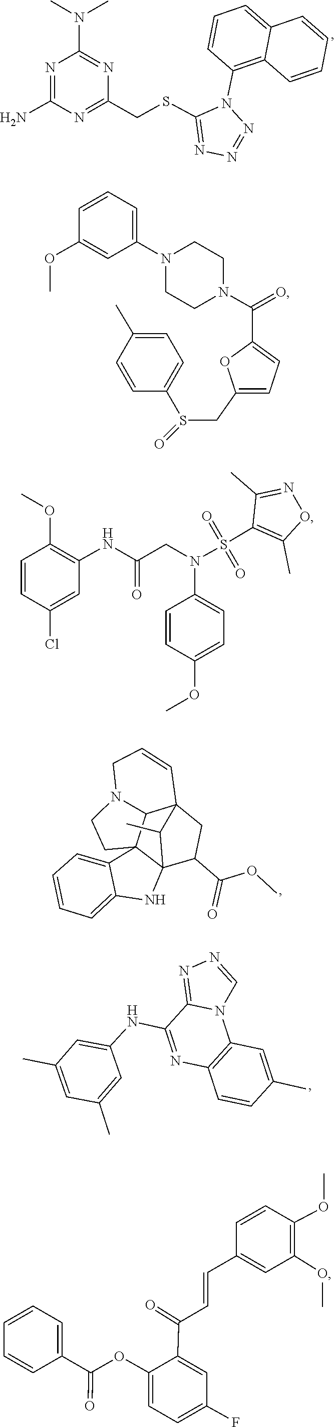 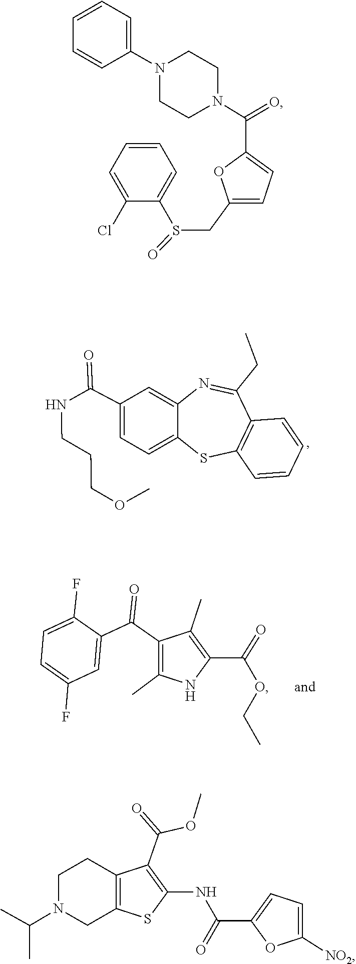 [SMILES: O=C(c1ccc(CS(=O)c2ccccc2Cl)o1)N1CCN(c2ccccc2)CC1 ; CCC1=Nc2cc(C(=O)NCCCOC)ccc2Sc2ccccc21 ; CCOC(=O)c1[nH]c(C)c(C(=O)c2cc(F)ccc2F)c1C ; COC(=O)c1c(NC(=O)c2ccc([N+](=O)[O-])o2)sc2c1CCN(C(C)C)C2]
or a pharmaceutically acceptable salt of any of the foregoing; wherein the compound, or a pharmaceutically acceptable salt thereof, is radiolabeled.

36. The method of claim 34 or 35, wherein the compound, or a pharmaceutically acceptable salt thereof, is selected from the group consisting of: 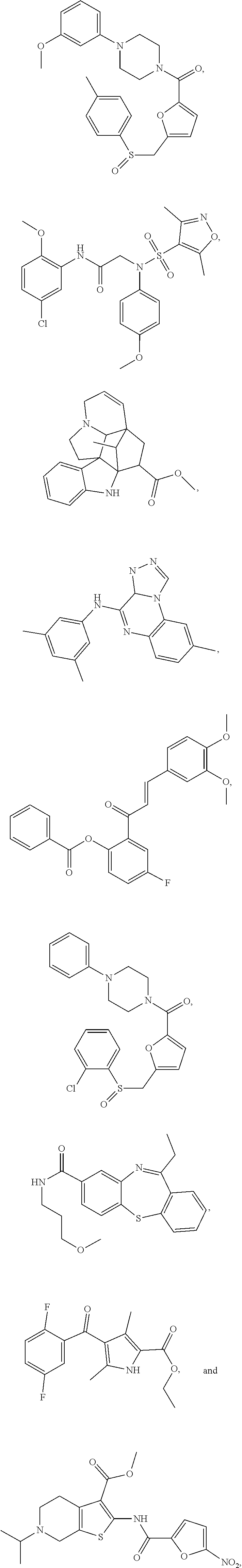
or a pharmaceutically acceptable salt of any of the foregoing.

37. The method of any one of claims 34-36, wherein the radiolabeled compound is 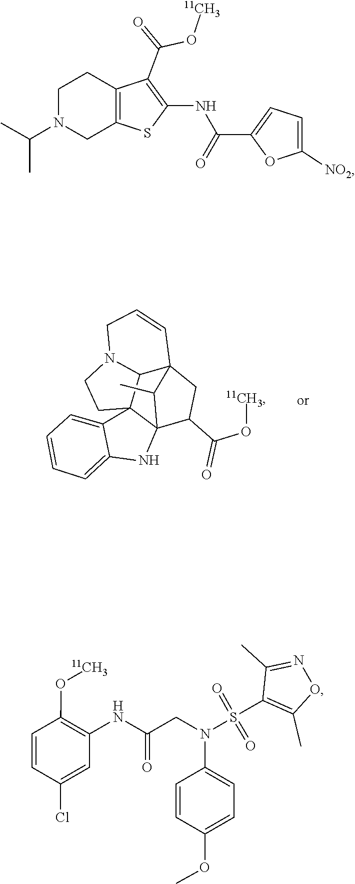
or a pharmaceutically acceptable salt of any of the foregoing.

38. A method for detecting α-synuclein translation in a mammalian cell, comprising contacting the mammalian cell with an effective amount of a compound selected from the group consisting of: 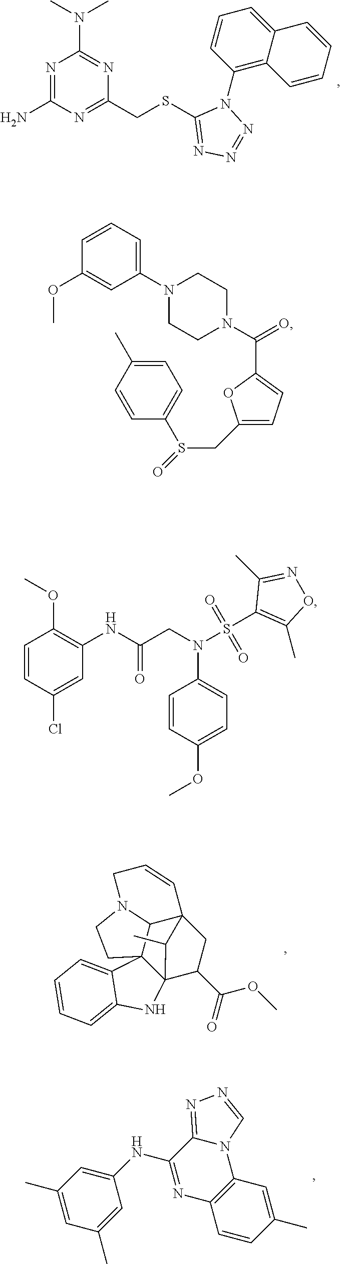 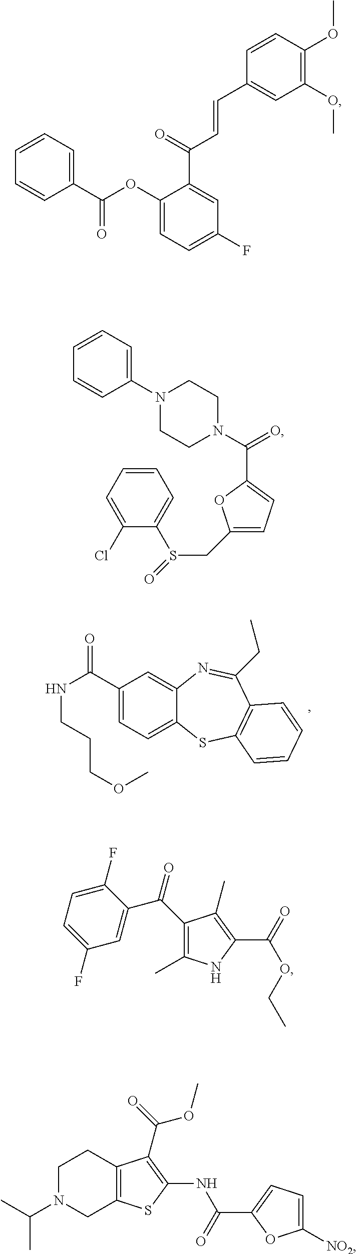 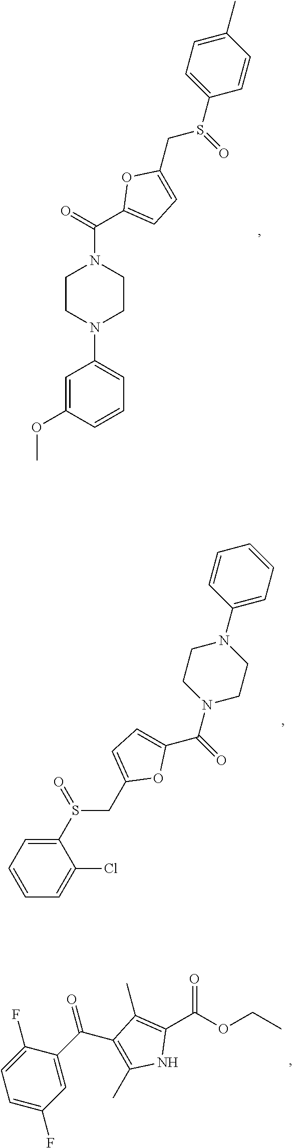 [SMILES: COc1cccc(N2CCN(C(=O)c3ccc(CS(=O)c4ccc(C)cc4)o3)CC2)c1 ; O=C(c1ccc(CS(=O)c2ccccc2Cl)o1)N1CCN(c2ccccc2)CC1 ; CCOC(=O)c1[nH]c(C)c(C(=O)c2cc(F)ccc2F)c1C] 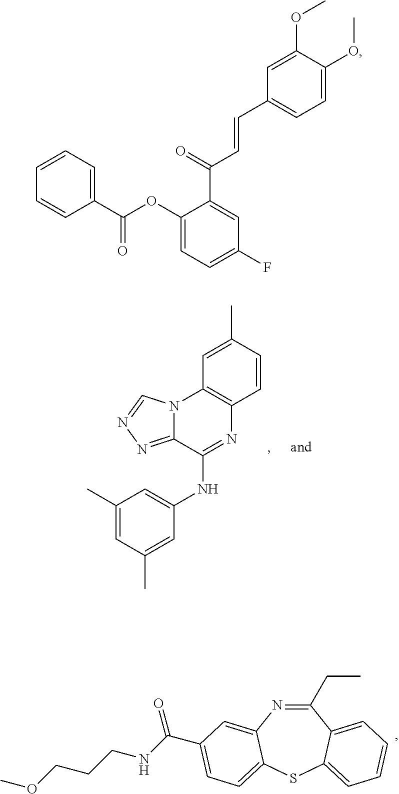
or a pharmaceutically acceptable salt of any of the foregoing; wherein the compound, or a pharmaceutically acceptable salt thereof, is radiolabeled.

39. A method for detecting α-synuclein translation in a mammalian cell, comprising contacting the mammalian cell with an effective amount of a compound selected from the group consisting of: 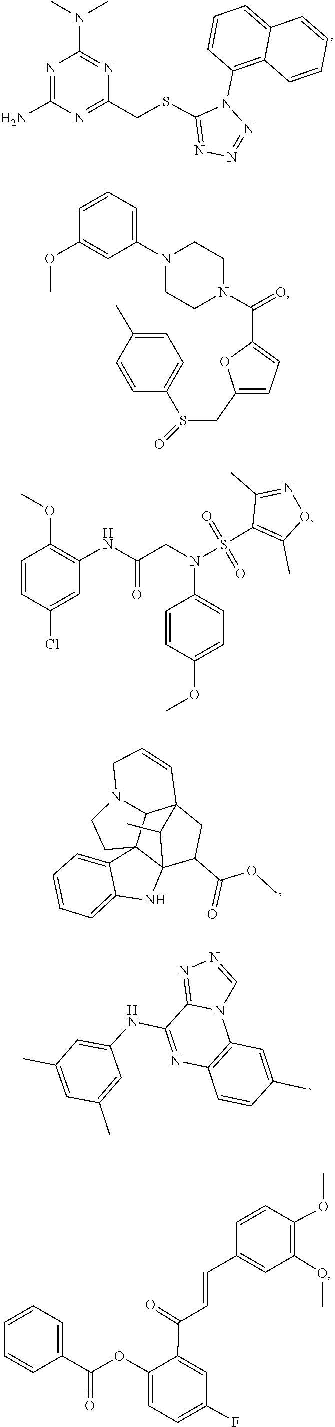 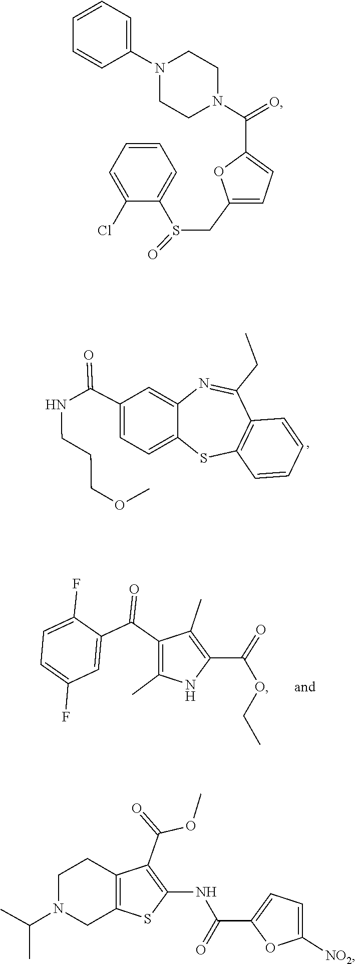 [SMILES: O=C(c1ccc(CS(=O)c2ccccc2Cl)o1)N1CCN(c2ccccc2)CC1 ; CCC1=Nc2cc(C(=O)NCCCOC)ccc2Sc2ccccc21 ; CCOC(=O)c1[nH]c(C)c(C(=O)c2cc(F)ccc2F)c1C ; COC(=O)c1c(NC(=O)c2ccc([N+](=O)[O-])o2)sc2c1CCN(C(C)C)C2]
or a pharmaceutically acceptable salt of any of the foregoing; wherein the compound, or a pharmaceutically acceptable salt thereof, is radiolabeled.

40. The method of claim 38 or 39, wherein the compound, or a pharmaceutically acceptable salt thereof, is selected from the group consisting of: 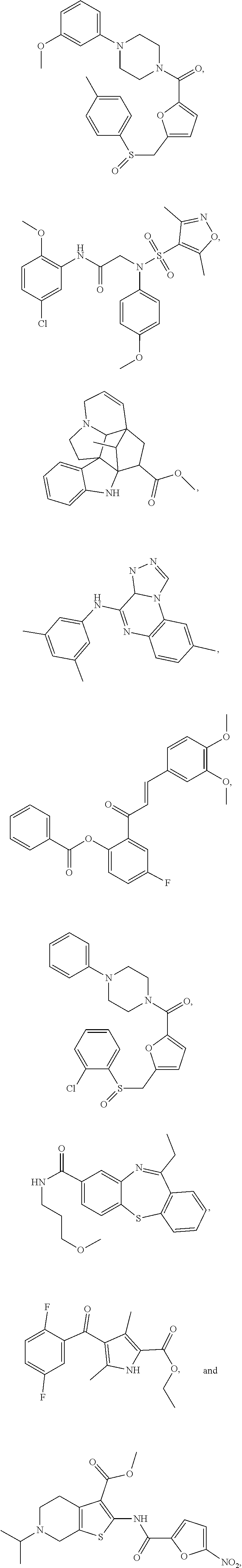
or a pharmaceutically acceptable salt of any of the foregoing.

41. The method of any one of claims 38-40, wherein the radiolabeled compound is 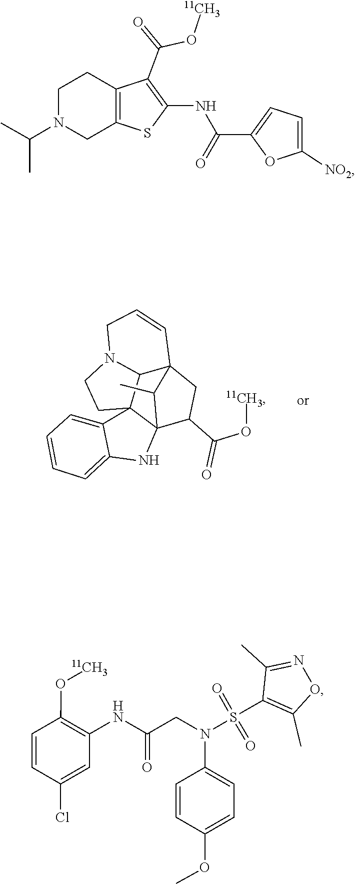
or a pharmaceutically acceptable salt of any of the foregoing.

42. A method for detecting Lewy body formation in mammalian brain tissue, comprising contacting the mammalian brain tissue with an effective amount of a compound selected from the group consisting of: 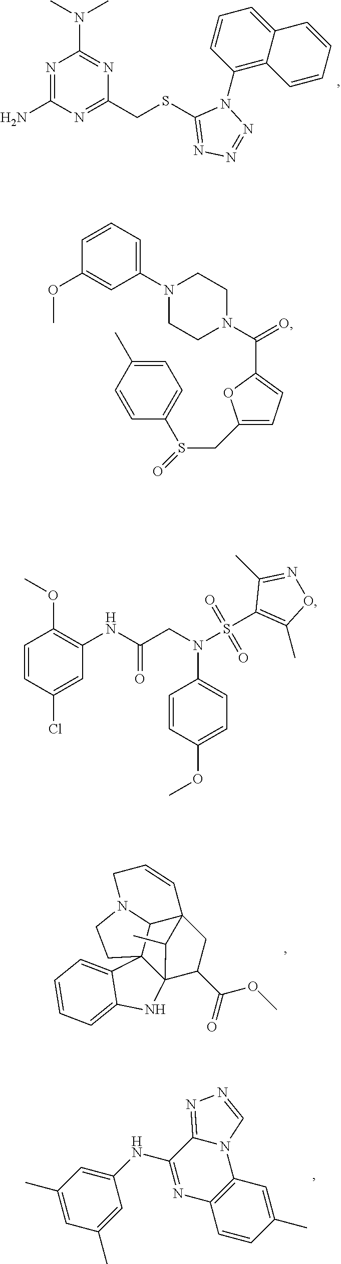 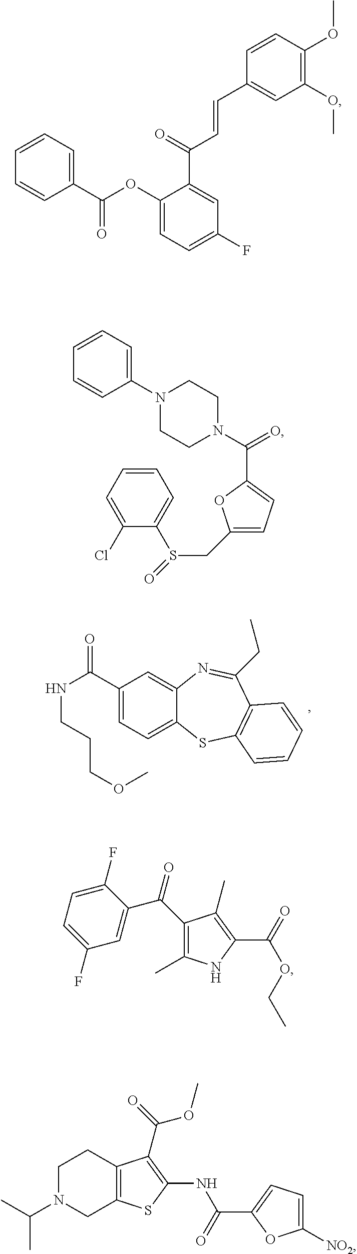 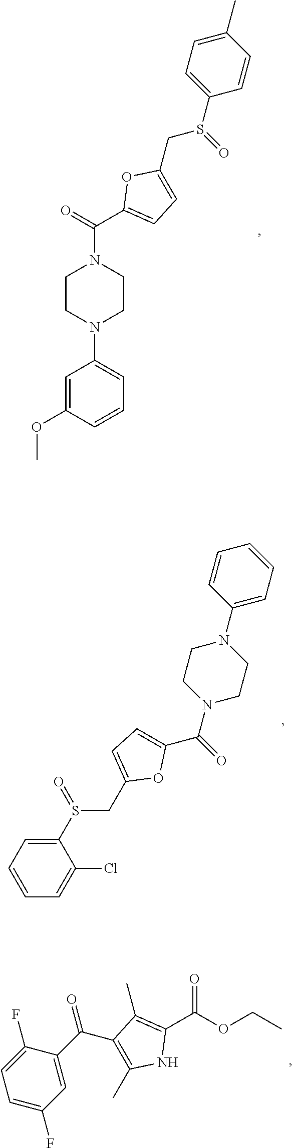 [SMILES: COc1cccc(N2CCN(C(=O)c3ccc(CS(=O)c4ccc(C)cc4)o3)CC2)c1 ; O=C(c1ccc(CS(=O)c2ccccc2Cl)o1)N1CCN(c2ccccc2)CC1 ; CCOC(=O)c1[nH]c(C)c(C(=O)c2cc(F)ccc2F)c1C] 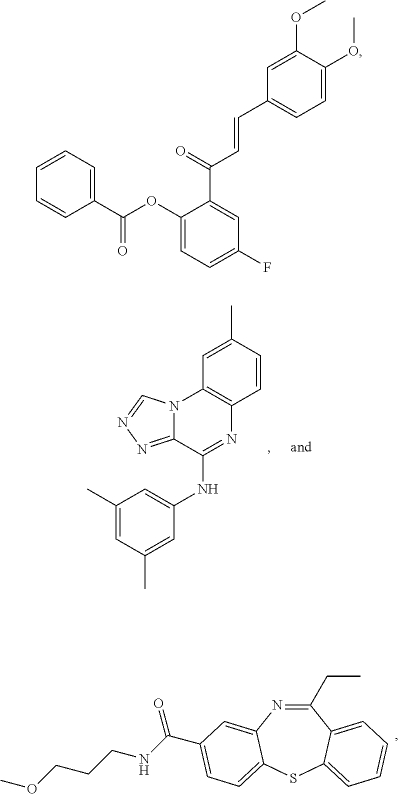
or a pharmaceutically acceptable salt of any of the foregoing; wherein the compound, or a pharmaceutically acceptable salt thereof, is radiolabeled.

43. A method for detecting Lewy body formation in mammalian brain tissue, comprising contacting the mammalian brain tissue with an effective amount of a compound selected from the group consisting of: 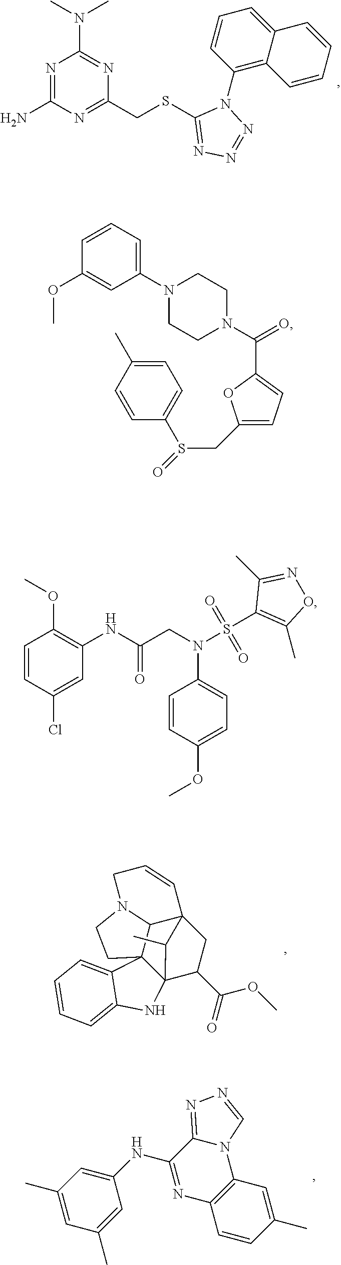 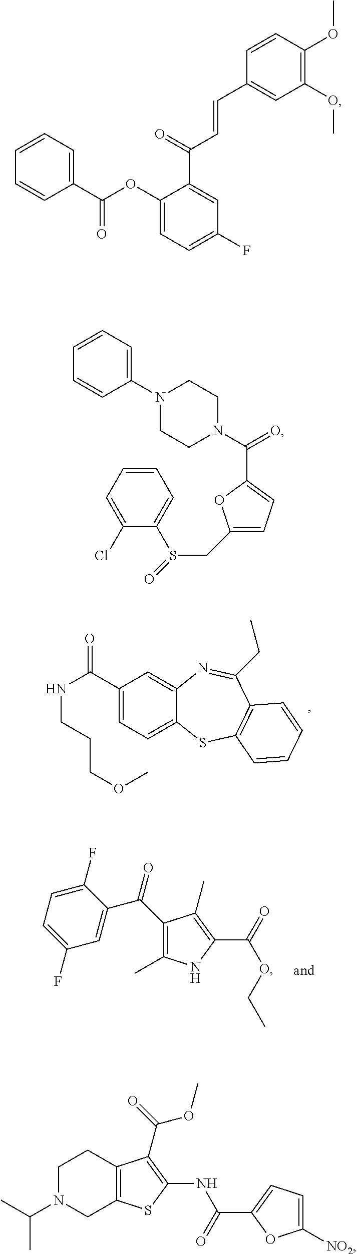
or a pharmaceutically acceptable salt of any of the foregoing; wherein the compound, or a pharmaceutically acceptable salt thereof, is radiolabeled.

44. The method of claim 42 or 43, wherein the compound, or a pharmaceutically acceptable salt thereof, is selected from the group consisting of: 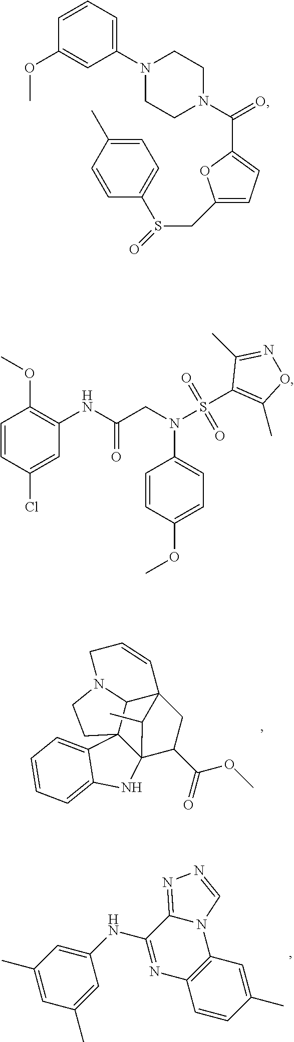 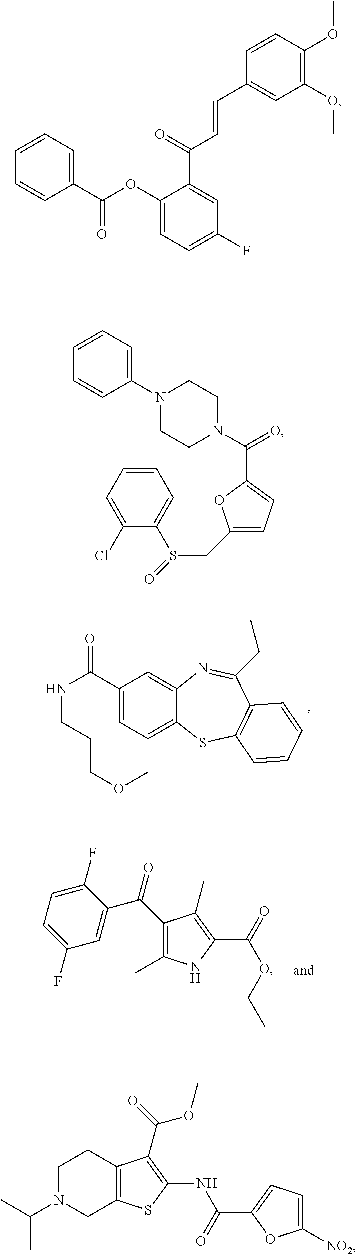
or a pharmaceutically acceptable salt of any of the foregoing.

45. The method of any one of claims 42-44, wherein the compound, or a pharmaceutically acceptable salt thereof, is selected from the group consisting of: 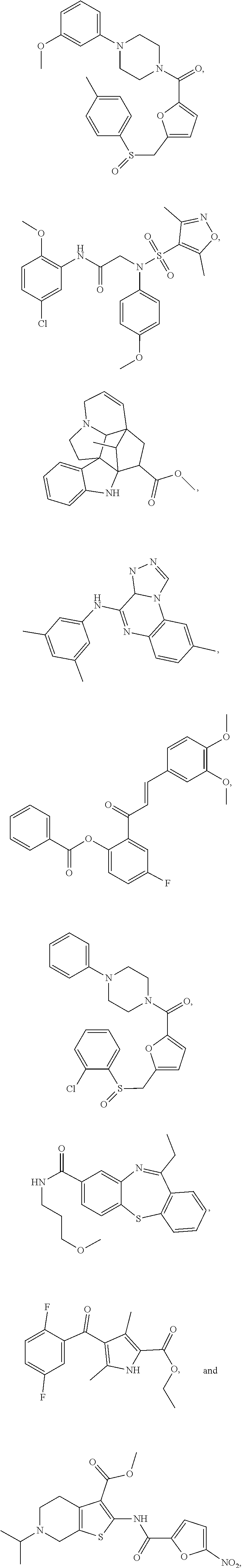
or a pharmaceutically acceptable salt of any of the foregoing.

46. The method of any one of any one of claims 42-45, wherein the radiolabeled compound is 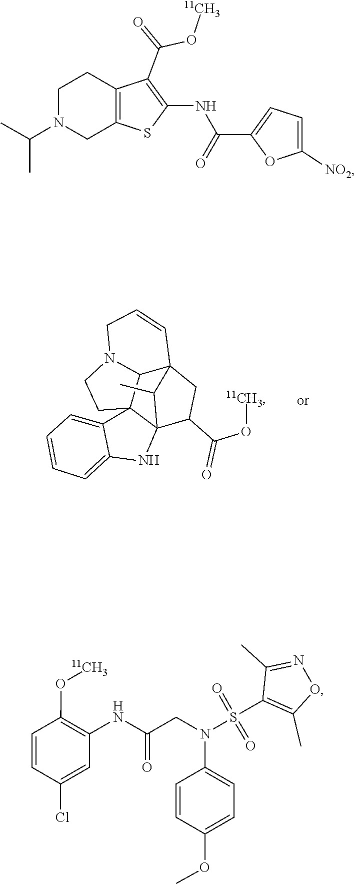
or a pharmaceutically acceptable salt of any of the foregoing.

47. An imaging composition comprising a compound selected from the group consisting of: 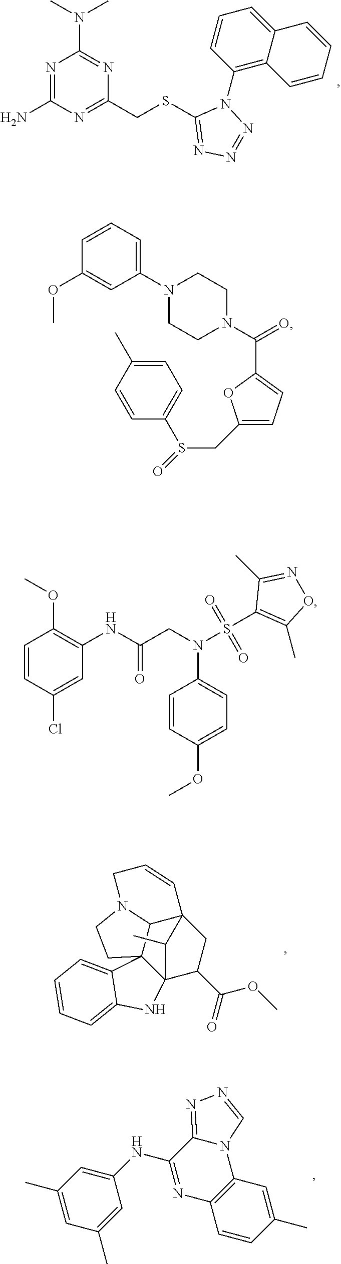 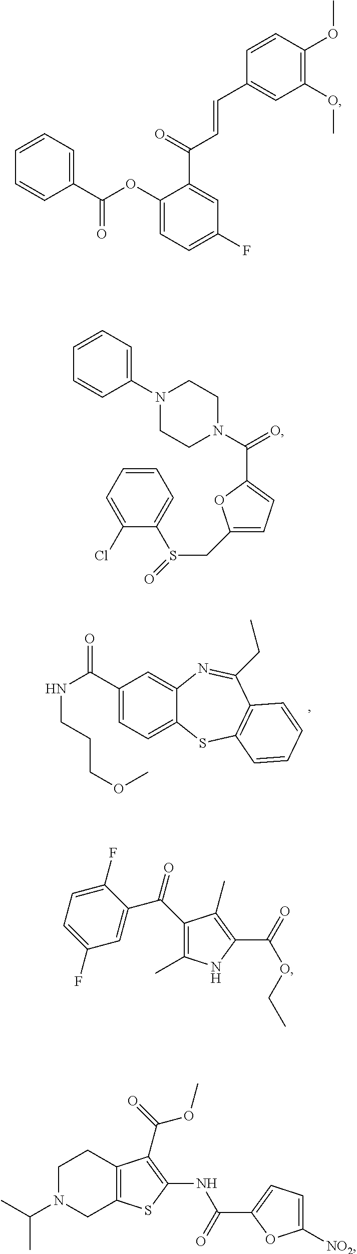 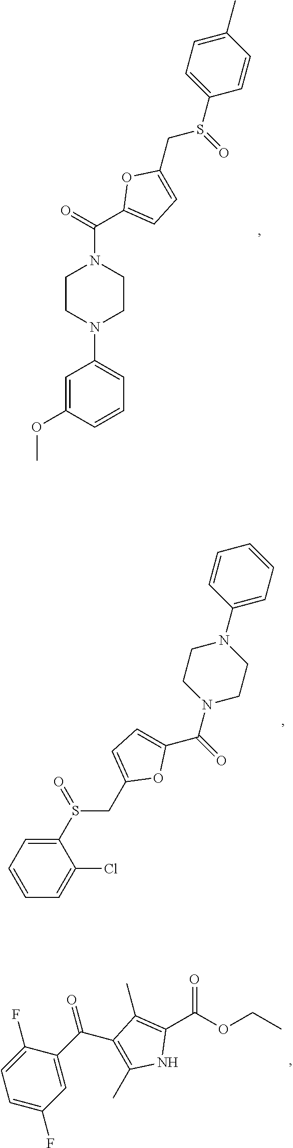 [SMILES: COc1cccc(N2CCN(C(=O)c3ccc(CS(=O)c4ccc(C)cc4)o3)CC2)c1 ; O=C(c1ccc(CS(=O)c2ccccc2Cl)o1)N1CCN(c2ccccc2)CC1 ; CCOC(=O)c1[nH]c(C)c(C(=O)c2cc(F)ccc2F)c1C] 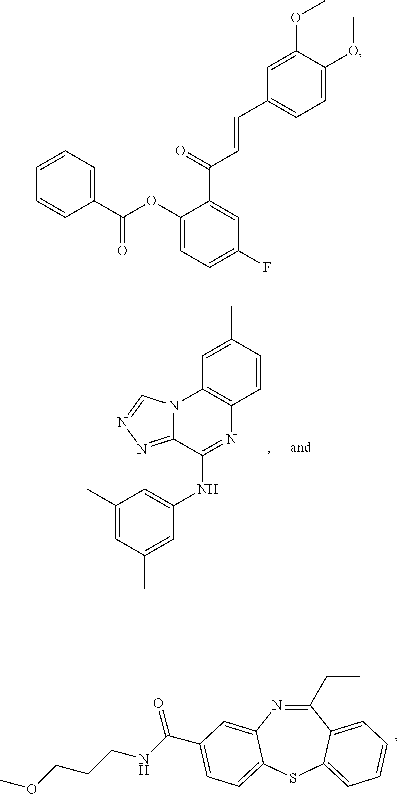
or a pharmaceutically acceptable salt of any of the foregoing, and one or more pharmaceutically acceptable excipients; wherein the compound, or a pharmaceutically acceptable salt thereof, is radiolabeled.

48. An imaging composition comprising a compound selected from the group consisting of: 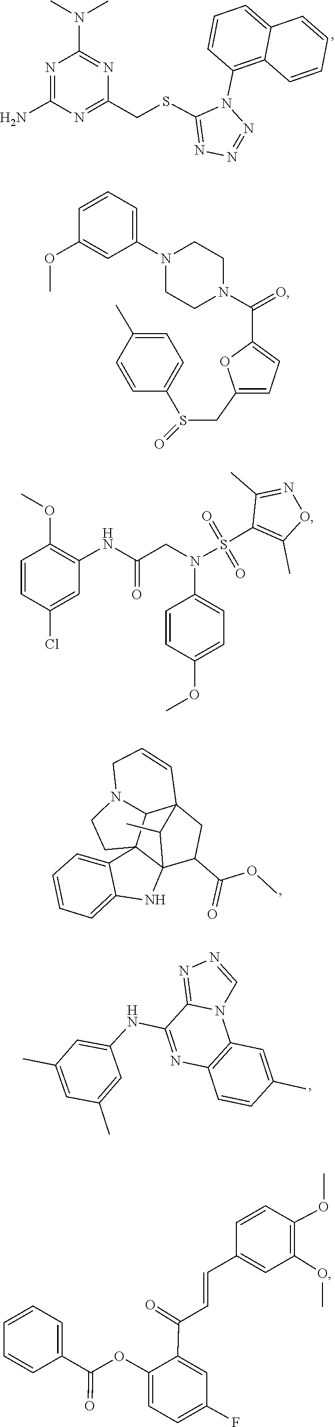 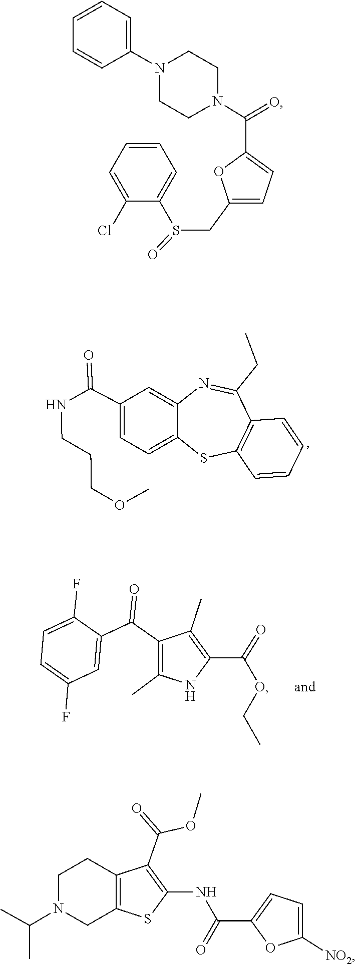 [SMILES: O=C(c1ccc(CS(=O)c2ccccc2Cl)o1)N1CCN(c2ccccc2)CC1 ; CCC1=Nc2cc(C(=O)NCCCOC)ccc2Sc2ccccc21 ; CCOC(=O)c1[nH]c(C)c(C(=O)c2cc(F)ccc2F)c1C ; COC(=O)c1c(NC(=O)c2ccc([N+](=O)[O-])o2)sc2c1CCN(C(C)C)C2]
or a pharmaceutically acceptable salt of any of the foregoing, and one or more pharmaceutically acceptable excipients; wherein the compound, or a pharmaceutically acceptable salt thereof, is radiolabeled.

49. The imaging composition of claim 47 or 48, wherein the compound, or a pharmaceutically acceptable salt thereof, is selected from the group consisting of: 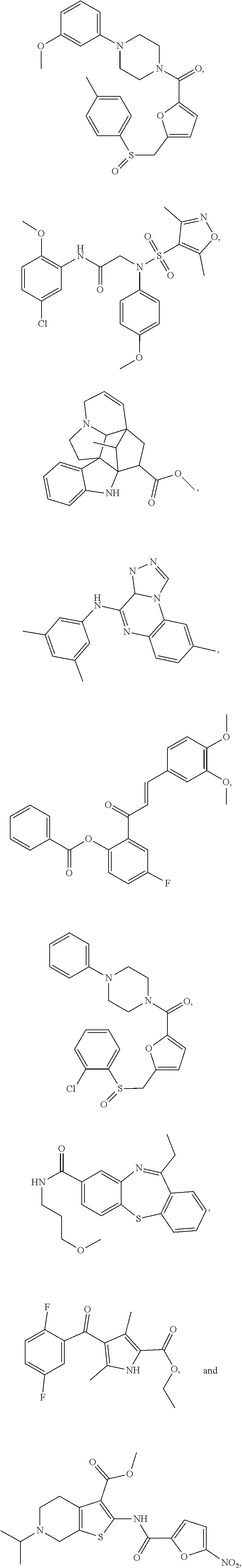
or a pharmaceutically acceptable salt of any of the foregoing.

50. The imaging composition of any one of claims 47-49, wherein the radiolabeled compound is 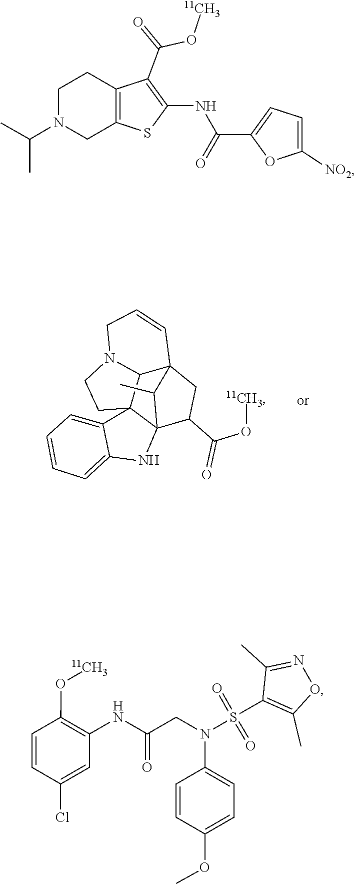
or a pharmaceutically acceptable salt of any of the foregoing.
